(19) United States
(12) Patent Application Publication
(10) Pub. No.: US 2016/0146806 A1
(43) Pub. Date: May 26, 2016

(54) RECEPTORS FOR B7-H4

(71) Applicant: AMPLIMMUNE, INC., Gaithersburgh, MD (US)

(72) Inventors: Solomon Langermann, Baltimore, MD (US); Joseph R. Podojil, Downers Grove, IL (US); Stephen D. Miller, Oak Park, IL (US)

(21) Appl. No.: 14/891,867

(22) PCT Filed: May 19, 2014
(86) PCT No.: PCT/US14/38623

§ 371 Date: Nov. 17, 2015

Related U.S. Application Data
(60) Provisional application No. 61/824,860, filed on May 17, 2013.

(51) Int. Cl. G01N 33/564; C12N 15/113; C07K 16/28; C07K 14/71; C07K 14/705

(52) U.S. Cl. CPC G01N 33/564; C07K 14/71; C07K 14/70532; C07K 16/2827; C12N 15/1138; G01N 2333/70532; G01N 2800/52; G01N 2800/24; C07K 2317/76; C07K 2319/30; G01N 2800/245; A61K 38/00

(57) ABSTRACT

Isolated cell surface receptors for B7-H4 have been identified based on function. B7-H4 receptor activation by B7-H4 on the dendritic cell, T follicular helper cell and germinal center B cell membrane stimulates inhibitory signaling in those leukocytes. B7-H4 receptor activation decreases production and/or secretion of proinflammatory cytokines, and promotes anti-inflammatory cytokine by mature DC and T cells. Modulators of B7-H4 receptor polypeptides and methods for their therapeutic use are also provided.

### FIELD OF THE INVENTION

This invention generally relates to receptors for B7-H4 and compositions and methods for modulating T-cell activation through B7-H4 receptors.

### BACKGROUND OF THE INVENTION

Antigen-specific activation and proliferation of lymphocytes are regulated by both positive and negative signals from co stimulatory molecules. The most extensively characterized T cell co stimulatory pathway is B7-CD28, in which B7-1 (CD80) and B7-2 (CD86) each can engage the stimulatory CD28 receptor and the inhibitory CTLA-4 (CD152) receptor. In conjunction with signaling through the T cell receptor, CD28 ligation increases antigen-specific proliferation of T cells, enhances production of cytokines, stimulates differentiation and effector function, and promotes survival of T cells (Lenshow, et al., Annu. Rev. Immunol., 14:233-258 (1996); Chambers and Allison, Curr. Opin. Immunol., 9:396-404 (1997); and Rathmell and Thompson, Annu. Rev. Immunol., 17:781-828 (1999)).

In contrast, signaling through CTLA-4 is thought to deliver a negative signal that inhibits T cell proliferation, IL-2 production, and cell cycle progression (Krummel and Allison, J. Exp. Med., 183:2533-2540 (1996); and Walunas, et al., J. Exp. Med., 183:2541-2550 (1996)). Other members of the B7 family include B7-H1 (Dong, et al., Nature Med., 5:1365-1369 (1999); and Freeman, et al., J. Exp. Med., 192:1-9 (2000)), B7-DC (Tseng, et al., J. Exp. Med., 193:839-846 (2001); and Latchman, et al., Nature Immunol., 2:261-268 (2001)), B7-H2 (Wang, et al., Blood, 96:2808-2813 (2000); Swallow, et al., Immunity, 11:423-432 (1999); and Yoshinaga, et al., Nature, 402:827-832 (1999)), B7-H3 (Chapoval, et al., Nature Immunol., 2:269-274 (2001)), B7-H4 (Choi, et al., J. Immunol., 171:4650-4654 (2003); Sica, et al., Immunity, 18:849-861 (2003); Prasad, et al., Immunity, 18:863-873 (2003); and Zang, et al., Proc. Natl. Acad. Sci. U.S.A., 100:10388-10392 (2003)), B7-H5 (Zhu, et al, Nature Comm., DOI: 10.1038/ncomms3043) and B7-H6 (Brandt, et al, J Exp Med. 2009 Jul. 6; 206(7):1495-503). B7-H1 and B7-DC are ligands for PD-1. B7-H2 is a ligand for ICOS. B7-H5 is a ligand for CD28H. B7-H6 is a ligand for NKp30, and B7-H3 remains an orphan ligand at this time (Dong, et al., Immunol. Res., 28:39-48 (2003)).

B7-H4 is a member of the B7 family and is a negative regulator of T cell responses. Human and mouse B7-H4 share 87% amino acid identity suggesting an important evolutionarily conserved function. Human and mouse B7-H4 mRNAs are expressed broadly in both lymphoid (spleen and thymus) and nonlymphoid organs (including lung, liver, testis, ovary, placenta, skeletal muscle, pancreas, and small intestine); however B7-H4 is low or not detected in most normal human tissues by immunohistochemistry. Limited studies of B7-H4 protein expression indicate that B7-H4 is not expressed on freshly isolated human T cells, B cells, DC, and monocytes, but it can be induced on these cell types after in vitro stimulation. Immunohistochemical staining shows that B7-H4 is highly expressed in lung, breast, and ovarian tumors, and real-time polymerase chain reaction (PCR) analyses indicate that mouse B7-H4 also is highly expressed in a number of tumor cell lines, including breast, prostate, lung, and colon carcinomas.

Functional studies using B7-H4 transfectants and immobilized B7-H4-Ig fusion proteins demonstrate that B7-H4 delivers a signal that inhibits TCR-mediated CD4⁺ and CD8⁺ T proliferation, cell-cycle progression and IL-2 production. Further support of the inhibitory role of B7-H4 comes from studies in which the blockade of B7-H4 by a blocking mAb against B7-H4 promoted allo-reactive CTL activity in a parental to -F1 GVHD model (Sica, et al., Immunity, 18:849-861(2003)). Additionally, a B7-H4 blocking mAb has been shown to aggravate disease progression in an EAE model of disease (Prasad, Immunity, 18:863-873 (2003)). B7-1 costimulation cannot overcome B7-H4-Ig-induced inhibition. In agreement with the in vitro activity, B7-H4 knock-out mice develop autoimmunity in certain mouse strains, with the severity of the disease inversely correlated to the levels of B7-H4 expression. The broad and inducible expression of B7-H4, together with functional studies, suggests that B7-H4 may serve to downregulate immune responses in peripheral tissues and play a role in promoting T cell tolerance.

Identification and characterization of cell surface receptors for B7-H4 is important for understanding of the mechanisms of B7-H4 regulation of immune functions and for the development of new therapies for the treatment of diseases and disorders related to these mechanisms.

Therefore, it is an object of the invention to provide B7-H4 receptors.

It is another object of the invention to provide molecules that modulate B7-H4 receptors and methods of use thereof to downregulate immune responses such as T cell activation.

It is another object of the invention to provide compositions that modulate B7-H4 receptors and methods of use thereof to reduce neutrophil proliferation or activation.

It is another object of the invention to provide compositions and methods to inhibit or reduce recruitment of immune cells including neutrophils, macrophages, and monocytes or other cells involved in inflammatory response.

It is another object of the invention to provide compositions that modulate B7-H4 receptors to block chemokine production and inhibit/attenuate recruitment of cells involved in inflammatory responses including blocking Th1, Th17 differentiation and production of associated cytokines, enhancement of IL-10, induction and enhanced activity of Tregs.

It is another object of the invention to provide compositions that modulate B7-H4 receptors to inhibit the function of germinal center associated T follicular helper cells and B cells.

It is another object of the invention to provide compositions that modulate B7-H4 receptors to regulate the function and cytokine production of pro-inflammatory macrophages, dendritic cells and monocytes.

It is another object of the invention to provide compositions and methods for the treatment of inflammatory responses, autoimmune disorders, and transplant rejection by targeting B7-H4 or B7-H4 receptors.

It is another object of the invention to provide molecules that inhibit, reduce or block B7-H4 receptors and methods of use thereof to maintain, prolong, or enhance T cell activation.

It is another object of the invention to provide compositions and methods for the treatment of infectious diseases and cancer.

It is another object of the invention to provide methods and compositions for inhibiting, reducing, or blocking the biological activity of soluble B7-H4. It is another object of the invention to provide methods for identifying antibodies and small molecules that modulate B7-H4 or B7-H4 receptor activity.

It is another object of the invention to provide methods for identifying neutralizing anti-B7-H4 antibodies and small molecules.

It is still another object of the invention to provide biomarkers for assessing the effectiveness of immunotherapies.

It is another object of the invention to provide compositions and methods for assisting in the diagnosis of an autoimmune or inflammatory disease/disorder, or cancer in a subject, or assessing the propensity for a subject to develop an autoimmune or inflammatory disease/disorder, or cancer.

It is a further object of the invention to provide compositions and methods for determining the severity of an autoimmune or inflammatory disease/disorder, or cancer in a subject having or suspected of having an autoimmune or inflammatory disease/disorder, or cancer.

It is another object of the invention to provide compositions and methods for determining the efficacy of a treatment for an autoimmune or inflammatory disease/disorder, or cancer.

It is another object of the invention to provide compositions and methods for selecting a subject for treatment for an autoimmune or inflammatory disease/disorder, or cancer.

It is another object of the invention to provide compositions and methods for measuring pharmacokinetic and pharmacodynamics parameters of B7-H4 therapies, such as soluble proteins that mimic transmembrane B7-H4.

### SUMMARY OF THE INVENTION

It has been discovered that activated leukocyte cell adhesion molecule neuropilin, Plexin4A, and complexes thereof are receptors for B7-H4. The neuropilin can be neuropilin 1 or neuropilin 2. The neuropilin can be a receptor for B7-H4 alone or neuropilin can be part of a complex of proteins that collectively act as a receptor for B7-H4. The complex proteins can include one or more of neuropilin, plexin, and a semaphorin. In still another embodiment, B7-H4 binds to or associates with neuropilin and semaphorin binds to or associates with Plexin4. The binding or association of semaphorin to plexin can enhance or increase the binding of B7-H4 to neuropilin.

In another embodiment, B7-H4 can bind to semaphorin and then the combination of B7-H4 binds to neuropilin.

In a particular embodiment, a recombinant B7-H4 receptor includes a neuropilin, wherein the receptor binds to a B7-H4 polypeptide including the amino acid sequence of any of SEQ ID NOS: 4-19 or a B7-H4-Ig fusion protein including the amino acid sequence of SEQ ID NO:22, 23, 24, or 25.

B7-H4 may interact with neuropilin as a monomer, homodimer or a heterodimer. In some embodiments, B7-H4 interacts with neuropilin via a C-type lectin or molecule that functions like a C-type lectin. The C-type lectin induces one or more conformational changes in B7-H4 that enables B7-H4 to interact with neuropilin. In a preferred embodiment, B7-H4 forms a heterodimer with or binds to neuropilin in the presence of a second ligand of neuropilin such as semaphorin or VEGF.

The Examples below show that Nrp-1 can form a co-receptor complex with PlexinA4 and the presence of PlexinA4 increases the binding affinity of B7-H4 for Nrp-1. Semaphorins, such as Semaphorin 3A (Sema3A) and Semaphorin 6C (Sema 6C), can form a co-ligand with B7-H4 and further stabilize the binding of B7-H4 to Nrp-1 within the receptor complex.

In healthy individuals without immune challenge, Nrp-1 is expressed on the surface of Treg cells (but not naïve Th cells and at low levels on a small proportion of pre-exiting self-antigen/tolerized T cells). Nrp-1 promotes prolonged interactions with immature DCs (imDCs) and gives Treg cells an advantage over naïve Th cells and antigen-specific T cell in the absence of proinflammatory stimuli. The binding of Tregs to imDCs is mediated by B7-H4 (and can be enhanced by association of Semaphorins with B7-H4) on the surface of the imDC, and the Nrp-1/PlexinA4 co-receptor on the surface of the Treg, within the immune synapse. Binding of the imDCs to Tregs results in higher sensitivity to limiting amounts of antigen compared to naïve Th cells with the same antigen specificity. Once activated, Treg cells suppress the activation and function of Th cells, B cells and DCs.

Proinflammatory signals promote the transition of imDCs to mature DCs (mDCs) and an increase in Nrp-1 expression on antigen specific Th cells including those previously tolerized to self-antigens. mDCs form more longer-term interactions with Th cells than imDCs shifting the balance of binding and activation in favor of Th cells. Such activated Th cells overcome the tolerogenic effects of Tregs.

In autoimmune conditions, activated Th cells overexpress Nrp-1/PlexinA4 (as evidenced by B7-H4 Ig binding), which gives them a competitive advantage over Tregs for binding to DCs. Therefore, binding of the Nrp-1+ Th cells to DCs results in higher sensitivity to limiting amounts of antigen compared to naïve Nrp-1− Th cells with the same antigen specificity. Activated Nrp-1+ Th cells can therefore bind to and activate DCs, overcoming Treg mediated tolerance, and prevent the re-establishment of tolerance. Activated Nrp-1+ Th cells mediate autoimmunity via the activation and maturation of DCs.

Therefore, compositions and methods of use thereof for inducing or enhancing immune stimulatory and immune inhibitory responses by modulating B7-H4 receptor signaling are disclosed. In some embodiments, the disclosed antagonists or agonists are targeted to cells expressing the B7-H4 receptor. Pharmaceutical compositions including one or more antagonists of a neuropilin, a plexin, or a complex thereof are disclosed. Typically, the antagonist inhibits, reduces, blocks or otherwise reduces binding or signal transduction between transmembrane B7-H4 and the B7-H4 receptor. In some embodiments, the antagonist disrupts binding or signal transduction between a B7-H4-Ig fusion protein and the B7-H4 receptor.

The antagonist can be a soluble B7-H4 receptor polypeptide or a fusion protein including a first domain that includes a soluble B7-H4 receptor polypeptide and a second domain. The second domain can be an Ig Fc region. An exemplary soluble B7-H4 receptor polypeptide is one that includes the extracellular domain of a neuropilin, a plexin, or a combination thereof. For example, the soluble B7-H4 receptor polypeptide can have 80% sequence identity to the extracellular domain of the neuropilin of SEQ ID NO:1 or the plexin of SEQ ID NO:2. In some embodiments, the soluble B7-H4 receptor polypeptide consists of the extracellular domain of SEQ ID NO:1 or SEQ ID NO:2 or a fragment thereof that can bind to B7-H4. An extracellular domain of SEQ ID NO:1 can be amino acids 22-856 of SEQ ID NO:1, or a functional fragment thereof. An extracellular domain of SEQ ID NO:2 can be 24-1894 of SEQ ID NO:2, or a functional fragment thereof.

The antagonist can be a soluble B7-H4 polypeptide, for example, a soluble B7-H4 polypeptide consisting of the extracellular domain of B7-H4 or a fragment or variant thereof. In a particular embodiment, the soluble B7-H4 polypeptide includes or consists of the IgV domain of B7-H4.

The antagonist can be an anti-B7-H4 antibody or an antigen binding fragment thereof; an anti-B7-H4 receptor antibody, such an antibody or an antigen binding fragment thereof, that binds to a neuropilin or a plexin; or a bi-specific antibody that targets B7-H4 and the B7-H4 receptor; or a combination thereof that reduces, inhibits or blocks signal transduction through the B7-H4 receptor. For example, the antibody can bind to a B7-H4 receptor polypeptide having at least 80% sequence identity to the extracellular domain of SEQ ID NO:1 or SEQ ID NO:2. In some embodiments, the antibody binds to the extracellular domain of SEQ ID NO:1 or SEQ ID NO:2 or a fragment thereof that can bind to B7-H4.

In some embodiments the antagonist is an antibody that binds to a co-ligand of B7-H4, for example a semaphorin such as Sema3A or Sema6C. In particular embodiments, the antibody or antigen binding fragment thereof binds to SEQ ID NO:40, or a variant thereof having at least 80% sequence identity to SEQ ID NO:40, or a functional fragment thereof. In another particular embodiment, the antibody or antigen binding fragment thereof binds to SEQ ID NO:43, or a variant thereof having at least 80% sequence identity to SEQ ID NO:43, or a functional fragment thereof.

In another embodiment, the antagonist of is an inhibitory nucleic acid that reduces expression of a nucleic acid encoding a neuropilin, a plexin, a semaphorin, or B7-H4.

An antagonist can be present in the compositions in an amount effective to induce, increase, or enhance an immune stimulatory response; or a combination thereof. The antagonist can be in a dosage effective to modulate the balance of proinflammatory and anti-inflammatory cytokines produced by dendritic cell, activated T cell or T follicular helper cell or other immune cell type. The antagonist can be in a dosage effective to break Treg mediated immune tolerance; to increase a T cell response, increase proliferation of T cells, differentiation or effector function of T cells or survival of T cells; to reduce or inhibit a regulatory T cell response, proliferation of regulatory T cells, differentiation or effector function of regulatory T cells or survival of regulatory T cells; or any combination thereof. The compositions can include two more antagonists Immunogenic compositions including a receptor antagonist and an antigen are also disclosed.

In some embodiments, the B7-H4 receptor agonists or antagonists disclosed herein bind with a co-receptor complex that includes a neuropilin. For example, Nrp-1 is known to serve as co-receptor for semaphorin 3A in combination with plexin. Therefore, in some embodiments, the B7-H4 receptor is a neuropilin co-receptor complex that includes neuropilin and a second receptor polypeptide such as a plexin. The complex can be a heterodimer. In some embodiments, the co-receptors are in close proximity but not dimerized.

Methods for treating elevated expression of B7-H4 or a B7-H4 receptor, or a disease associated therewith, methods for enhancing Th1 and Th17 responses in a subject and methods for inducing, enhancing, maintaining or prolonging an immune response in a subject are disclosed. Methods for treating cancer and infectious diseases are also disclosed. The methods typically include administering to the subject an effective amount of a B7-H4 receptor antagonist or a pharmaceutical composition thereof. The subject can have an infection, for example an infection due to a virus, bacteria, fungus, or protozoa; or the subject can have cancer, such as bladder, brain, breast, cervical, colo-rectal, esophageal, kidney, liver, lung, nasopharangeal, pancreatic, prostate, skin, stomach, uterine, ovarian, and testicular cancer. In a particular embodiment, a method of treating cancer in a subject includes administering to the subject an effective amount of the B7-H4 receptor antagonist to inhibit tumor cell mediated immune suppression, increase or induce apoptosis of tumor cells, reduce or inhibit tumor cell proliferation, reduce or inhibit tumor cell migration, or a combination thereof. In some embodiment, the antagonist can reduce or inhibit the proliferative pathway, for example, the Erk1/2 pathway in tumor cells.

B7-H4 expressed on the surface of tumor cells (and infiltrating DCs) can bind to neuropilin receptor complexes on Tregs in the tumor microenvironment, leading to immune suppression. Antagonists of B7-H4 or neuropilin receptor complexes can overcome this immune suppression by inhibiting the interaction of tumor cells or DCs with Tregs and thus break immune tolerance, allowing for the activation of Th cells and the enhancement of effector immune functions.

B7-H4 receptor antagonists can be used in combination for enhanced efficacy. Some embodiments include two or more antagonists. For example, anti-Nrp-1 and anti-B7-H4 antibodies can be combined, or as a bi-specific antibody, such that anti-Nrp-1 targets Tregs and blocks immune evasion, whereas anti-B7-H4 can target the tumor cell directly. In a preferred embodiment, the anti-B7-H4 antibody has direct anti-tumor activity such as ADCC, CDC or ADC.

In another embodiment, receptor proteins can be used to target B7-H4+ tumors, or other cell types expressing B7-H4 such as tumor associated macrophages. Such receptor proteins may have direct anti-tumor activity or be modified to have anti-tumor activity.

Pharmaceutical compositions including one or more agonists of the B7-H4 receptors are also disclosed. The agonist can be a B7-H4 fusion protein, for example a fusion protein including a first domain including a soluble B7-H4 polypeptide and a second domain. The second domain can be Ig Fc region. The soluble B7-H4 polypeptide can include all or part of the extracellular domain of B7-H4, and preferably includes or consists of the IgV domain of B7-H4, the IgC domain of B7-H4, or a combination thereof. In a preferred embodiment, the B7-H4 Ig fusion protein is a dimer. Exemplary fusion proteins include SEQ ID NO:20, 21, 22, 23, 24, and 25.

The agonist can be an anti-B7-H4 receptor antibody or an antigen binding fragment thereof; a bi-specific antibody that targets B7-H4 and the B7-H4 receptor and enhances the interaction; or a combination thereof that induces, increases, or enhances signal transduction through the B7-H4 receptor. In some embodiments, the antibody or antigen binding fragment thereof binds to the extracellular domain of SEQ ID NO:1 or SEQ ID NO:2 or variant thereof comprising at least 80% sequence identity to the extracellular domain of SEQ ID NO:1 or SEQ ID NO:2.

The agonist can be a semaphorin, or variant, functional fragment, or fusion thereof, preferably in combination with B7-H4-Ig fusion protein. In preferred embodiments the semaphorin is Sema3A or Sema6C, or a functional fragment thereof. In some embodiments, the semaphorin has at least 80% sequence identity to SEQ ID NO:40-43, or 62. In preferred embodiments, the semaphorin does not include the signal sequence. In a particular embodiment, the agonist is a semaphorin fusion protein such as an Ig fusion protein.

The agonist can be in a dosage effective to increase an immune inhibitory response; to decrease an immune stimulatory response; or a combination thereof. The agonist can be in an amount effective to decrease the function and cytokine production of proinflammatory mature dendritic cell, suppress T cell response, proliferation of T cells, differentiation or effector function of T cells or survival of T cells, suppress the function and cytokine production of follicular T helper cell; inhibit the differentiation and antibody production of germinal center B cell; promote or increase a regulatory T cell response, proliferation of regulatory T cells, differentiation or effector function of regulatory T cells or survival of regulatory T cells; or any combination thereof. The compositions can include two or more agonists.

Methods for treating or inhibiting one or more symptoms of an inflammatory response in a subject in need thereof are also disclosed. The methods typically include administering to the subject an effective amount of a receptor agonist or pharmaceutical composition thereof. The inflammatory response can be associated with an autoimmune disease or disorder. The autoimmune disease can be rheumatoid arthritis, systemic lupus erythematosus, alopecia areata, anklosing spondylitis, antiphospholipid syndrome, autoimmune addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, or Wegener's granulomatosis.

In some embodiments, such agonists are used in the treatment of autoimmune disease by agonizing the activity of Treg resulting in signal transduction and suppressive function of Tregs, and the establishment of immune tolerance. In a preferred embodiment such agonists bind preferentially to and/or agonize Nrp-1 receptor complexes on the surface of Treg cells. In one embodiment the Nrp-1 receptor complex agonist is a B7-H4 fusion protein. In a preferred embodiment the Nrp-1 receptor complex agonist is a fusion protein comprising the extracellular domain of B7-H4 fused to the Fc region of an immunoglobulin protein.

Method for reducing or inhibiting transplant rejection in a subject in need thereof and methods of treating one or more symptoms of graft versus host disease (GVHD) in a subject including administering to the subject an effective amount of a B7-H4 receptor agonist are also disclosed.

Methods of determining the level of a B7-H4 receptor polypeptide or ligand thereof in a biological sample using B7-H4 receptor or ligand specific antibodies are also disclosed. Methods for detecting or quantifying proteins in a sample are known in the art. Such methods include, but are not limited to electrophoresis, chromatography, mass spectroscopy, and immunoassays. For example, in some embodiments, a biological sample such as serum or plasma, or cell of a subject obtained from a subject is subjected to an immunoassay, wherein the immunoassay includes contacting the biological sample with at least one B7-H4 receptor or ligand-specific antibody or antigen-binding fragment thereof such as a F(ab′)2 fragment, and detecting the antibody or fragment. Preferred immunoassays include, but are not limited to, radioimmunoassays, ELISAs, immunoprecipitation assays, Western blot, fluorescent immunoassays, and immunohistochemistry. In some embodiments, the level of cell-free B7-H4 in a biological sample is additionally or alternatively measured. In alternative embodiment, the levels of receptors, ligands, and/or B7-H4 polypeptides can be measured using mass spectroscopy.

A particularly preferred immunoassay is ELISA. ELISA typically includes the use of two different specific antibodies: a capture antibody and a detection antibody. In some embodiments an antibody or antigen binding fragment thereof that recognizes a B7-H4 receptor is used to capture most or all of the B7-H4 receptor in the sample. A detection antibody that can recognize most or all of the B7-H4 receptor can be used to determine the total level of B7-H4 receptor in the biological sample. In some embodiments, the detection antibody recognizes a different domain or epitope than the capture antibody. In some embodiments, the detection antibody recognizes a ligand bound to the receptor, for example a soluble or cell-free B7-H4 or a B7-H4 fusion protein. Therefore in some embodiments, the detection antibody binds to B7-H4, particularly the extracellular domain of B7-H4, or the second polypeptide of the fusion protein. For example, if the second polypeptide of the fusion protein is the Fc region for human IgG1, the antibody can be an anti-human IgG1 Fc antibody. In this way, receptor occupancy of therapeutic B7-H4 fusion protein or cell-free B7-H4 can be determined. In some embodiments, a fusion protein can be distinguished from cell-free B7-H4 only, transmembrane B7-H4 only, or a combination thereof.

Methods of detecting B7-H4 receptors and ligands can be applied in a number of diagnostic assays. For example, methods for determining the severity of an immune response, inflammatory or autoimmune disease/disorder, or cancer; methods for assisting in the diagnosis of an inflammatory or autoimmune disease/disorder or cancer; or assessing the propensity for developing an inflammatory or autoimmune disease/disorder, or cancer; methods for determining the efficacy of a treatment for an immune response, inflammatory or autoimmune disease/disorder, or cancer; methods for selecting a subject for treatment of an immune response, inflammatory or autoimmune disease/disorder, or cancer; and methods for determining the efficacy of a treatment for an immune response, inflammatory or autoimmune disease/disorder, or cancer in a subject are disclosed. In some embodiments, the methods include additional step(s) of treating the subject for the disease/disorder.

### BRIEF DESCRIPTION OF THE DRAWINGS

FIGS. 1A-1E are a series of bar graphs showing the binding of different concentrations (1 μg/ml, 0.1 μg/ml, 0.01 μg/ml, or 0.001 μg/ml) of various coating proteins or combinations thereof: human Nrp-1 (——), human Nrp-1+Plexin A4 (—▪—), human Nrp-1+human SemaC6 (—▴—), or human Nrp-1+mouse SemaC6 (—▾—) (1A); mouse Nrp-1 (——), mouse Nrp-1+Plexin A4 (—▪—), mouse Nrp-1+human SemaC6 (—▴—), or mouse Nrp-1+mouse SemaC6 (—▾—) (1B); Plexin A4 (——), human Nrp-1+Plexin A4 (—▪—), or mouse Nrp-1+PlexinA4 (—▴—) (1C); mouse Sema6C (——), human Nrp-1+mouse Sema6C (—▪—), or mouse Nrp-1+mouse Sema6C (—▴—) (1D); human Sema6C (——), human Nrp-1+human Sema6C (—▪—), or mouse Nrp-1+human Sema6C (—▴—) (16) bound to plate-bound human B7-H4-Ig-biotin (OD 450 nm).

FIGS. 2A-2D are a series of bar graphs. FIG. 2A shows the concentration of human B7-H4-Ig alone; human B7-H4-Ig in combination with human Sema6C; or human B7-H4-Ig in combination with human PlexinA4 bound to various concentrations (1.0 μg/ml, 0.5 μg/ml, 0.25 μg/ml, 0.125 μg/ml, 0.0625 μg/ml, 0.0313 μg/ml, 0.0156 μg/ml, 0.0078 μg/ml, 0.0039 μg/ml, and 0 μg/ml) of coating protein human Nrp-1. FIG. 2B shows the concentration of human Nrp-1 alone; human Nrp-1 in combination with human PlexinA4; or human Nrp-1 in combination with human Sema6C bound to various concentrations (1.0 μg/ml, 0.5 μg/ml, 0.25 μg/ml, 0.125 μg/ml, 0.0625 μg/ml, 0.0313 μg/ml, 0.0156 μg/ml, 0.0078 μg/ml, 0.0039 μg/ml, and 0 μg/ml) to coating protein human B7-H4-Ig. FIG. 2C shows the concentration of human B7-H4-Ig alone; human B7-H4-Ig in combination with mouse Sema6C; or human B7-H4-Ig in combination with human PlexinA4 bound to various concentrations (1.0 μg/ml, 0.5 μg/ml, 0.25 μg/ml, 0.125 μg/ml, 0.0625 μg/ml, 0.0313 μg/ml, 0.0156 μg/ml, 0.0078 μg/ml, 0.0039 μg/ml, and 0 μg/ml) to coating protein mouse Nrp-1. FIG. 2D shows the concentration of mouse Nrp-1 alone; mouse Nrp-1 in combination with human PlexinA4; or mouse Nrp-1 in combination with human Sema6C bound to various concentrations (1.0 μg/ml, 0.5 μg/ml, 0.25 μg/ml, 0.125 μg/ml, 0.0625 μg/ml, 0.0313 μg/ml, 0.0156 μg/ml, 0.0078 μg/ml, 0.0039 μg/ml, and 0 μg/ml) to coating protein human B7-H4-Ig.

FIG. 3 is a concentration-response curve showing binding of B7-H4-Ig (◯), B7-DC-Ig (□), and control Ig (Δ) to Sema3a.

FIGS. 4A-4B are line graphs showing the binding of various concentrations of B7-H4-Ig (4A) or B7-DC-IG (4B) binding to Nrp-1 (——), PlexinA4a (—▪—), Sema3a (—▴—), and Nrp-1+PlexinA4 (—▾—) in the presence of PBS+5% BSA alone; or Block+Sema3a (—Δ—), Nrp-1+Sema3a (—◯—), PlexinA4+Sema3a (—□—), or Nrp-1+PlexinA4+Sema3a (—♦—) in the presence of soluble Sema3a (1 μg/mL) in PBS+5% BSA.

FIGS. 5A-5D are line graphs showing the effect of increasing concentrations of B7-H4-Ig (μg/ml) on anti-CD3-induced proliferation (ΔCPM) (5A), IFN-γ secretion (pg/ml) (5B), IL-17 secretion (pg/ml) (5C), and IL-10 secretion (5D) (pg/ml) of wildtype (——) and Np-1 knockout (—Δ—) T cells.

FIG. 6 is a bar graph showing the concentration (pg/ml) of IFN-γ, IL-17, GM-CSF, IL-4, and IL-10 secreted into culture by wildtype (A and B) or Nrp-(−/−) (C and D) T cells treated with control Ig (A and C) or B7-H4-Ig (B and D).

FIGS. 7A-7D are line graphs showing the effect of increasing concentrations of B7-H4-Ig (μg/ml) on anti-CD3-induced GM-CSF secretion (pg/ml) (7A), IFN-γ secretion (pg/ml) (7B), IL-17 secretion (pg/ml) (7C), and IL-10 secretion (pg/ml) (7D) of wildtype NOD (——) and Np-1 knockout NOD (—Δ—) T cells.

FIG. 8A is a line graph showing the effect of increasing concentrations of control Ig (“a”), B7-H4-Ig (“b”), Nrp-1 (“c”), or Nrp-1 (2.5 μg/ml)+B7-H4-Ig (“d”) on T cell proliferation (ΔCPM). FIG. 8B is a line graph showing the effect of increasing concentrations of control Ig (“a”), B7-H4-Ig (“b”), PlexinA4 (“c”), or PlexinA4 (2.5 μg/ml)+B7-H4-Ig (“d”) on T cell proliferation (ΔCPM).

FIGS. 9A-9D are line graphs showing the effect of increasing concentrations of control Ig (μg/ml) (“a”), B7-H4-Ig (μg/ml) (“b”), control Ig (μg/ml)+Sema3A (1 μg/ml) (“c”), B7-H4-Ig (μg/ml)+Sema3A (1 μg/ml) (“d”) on anti-CD3-induced T cell proliferation (ΔCPM) (9A), IFN-γ secretion (pg/ml) (9B), IL-17 secretion (pg/ml) (9C), and IL-10 secretion (pg/ml) (9D).

FIGS. 10A-10D are line graphs showing the effect of increasing concentrations of control Ig (μg/ml), B7-H4-Ig (μg/ml), control Ig (μg/ml)+Sema3A (1 μg/ml), B7-H4-Ig (μg/ml)+Sema3A (1 μg/ml) on anti-CD3-induced T cell GM-CSF (pg/ml) (10A) secretion, IFN-γ secretion (pg/ml) (10B), IL-17 secretion (pg/ml) (10C), and IL-10 secretion (pg/ml) (10D).

FIGS. 11A-11D are single-parameter histograms showing Fluorescence Minus One (FMO) control and B7-H4-Ig binding to Nrp-1+/Sema3a− (11A), Nrp-1+/Sema3a+ (11B), Nrp-1−/Sema3a+ (11C), and Nrp-1−/Sema3a− (11D) cells.

FIG. 12 is a diagram including a dot-plot and four single-parameter histograms corresponding to B7-H4-Ig binding to CD4+ T cells in each of the four, gated quadrants of the dot-plot.

FIG. 13 is a diagram showing a series of dot-plots defining the gating of cells of a higher FSC population, and a series of corresponding single-parameter histograms each showing no further increase in the percentage of Sema3a+ cells following a rSema3a binding step, and a corresponding significant percentage of B7-H4Ig binding cells within the gated population of Sema3a+ cells.

FIG. 14 is a diagram showing a series of dot-plots defining the gating of cells of a lower FSC population, and a series of corresponding single-parameter histograms each showing no further increase in the percentage of Sema3a+ cells following a rSema3a binding step, and a corresponding significant percentage of B7-H4Ig binding cells within the gated population of Sema3a+ cells.

FIG. 15 is a line graph showing 293 cell proliferation in the presence of increasing amount of exogenous recombinant human Sema3a (μg/ml) and soluble B7-H4-Ig: (0 μg/ml, ——), (1 μg/ml, —▪—), (5 μg/ml, —▴—), and (10 μg/ml, —▾—).

### DETAILED DESCRIPTION OF THE INVENTION

### I. Definitions

As used herein the term “modulate” relates to a capacity to alter an effect or result.

The term “cell-free B7-H4,” also referred to herein as circulating forms of B7-H4, and sH4, includes soluble, monomeric B7-H4 polypeptides that are derived from endogenous transmembrane B7-H4. Cell-free B7-H4 typically includes the extracellular domain of B7-H4 or a biologically active fragment thereof. Human and mouse B7 proteins contain short intracytoplasmic domains, a single transmembrane domain and an extracellular domain. The extracellular domain typically contains two Ig domains; a membrane proximal IgC domain and a membrane distal IgV domain. The term cell-free B7-H4 encompasses any polypeptide fragment of B7-H4 that is shed or cleaved from a transmembrane form of B7-H4 produced by cells in vivo. Cell-free B7-H4 can be approximately 50-kDa by Western blot analysis, a size equal to the entire extracellular domain of a monomeric B7-H4 molecule in denatured condition. Cell-free B7-H4 can circulate systemically within a subject, can be localized to a tissue or microenvironment, or a combination thereof. For example, cell-free B7-H4 can be localized, or increased at a site of inflammation or around a tumor.

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein, the term “polypeptide” refers to a chain of amino acids of any length, regardless of modification (e.g., phosphorylation or glycosylation).

As used herein, the term “B7-H4 receptor” refers to a molecule present on a cell surface that binds to B7-H4.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences.

As used herein, an “expression control sequence” is a DNA sequence that controls and regulates the transcription and/or translation of another DNA sequence.

“Operably linked” refers to an arrangement of elements wherein the components so described are configured so as to perform their usual or intended function. Thus, two different polypeptides operably linked together retain their respective biological functions while physically linked together.

As used herein, “valency” refers to the number of binding sites available per molecule.

As used herein, the term “host cell” refers to prokaryotic and eukaryotic cells into which a recombinant expression vector can be introduced.

As used herein, “transformed” and “transfected” encompass the introduction of a nucleic acid (e.g. a vector) into a cell by a number of techniques known in the art.

As used herein, the phrase that a molecule “specifically binds” to a target refers to a binding reaction which is determinative of the presence of the molecule in the presence of a heterogeneous population of other biologics. Under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, the terms “immunologic”, “immunological” or “immune” response is the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

An “immunogenic agent” or “immunogen” is capable of inducing an immunological response against itself on administration to a mammal, optionally in conjunction with an adjuvant.

The terms “individual”, “host”, “subject”, and “patient” are used interchangeably herein, and refer to a mammal, including, but not limited to, humans, rodents such as mice and rats, and other laboratory animals.

As used herein, the term “polypeptide” refers to a chain of amino acids of any length, regardless of modification (e.g., phosphorylation or glycosylation).

### II. Receptors and Co-Ligands for B7-H4

It has been discovered that neuropilins, particularly neuropilin-1, alone or in combination with a plexin such as Plexin4A is a receptor for B7-H4. The B7-H4 ligand may function alone or in combination with a C-type lectin such as a semphorin.

As discussed in Prud'homme, et al., Oncotarget, 3:921-939 (2012), neuropilins (Nrps) are multifunctional singlepass transmembrane proteins that play an important role in development, immunity and cancer. Neuropilin-1 (Nrp1), and its homologue neuropilin-2 (Nrp2), are cell surface receptors that enhance cellular responses to several growth factors and other mediators under physiological and pathological conditions. They are expressed by endothelial cells, several other normal cell types, and often by malignant tumor cells. Nrp1 and Nrp2 have 44% homology and share many structural and biological properties. Nrps are usually expressed as homodimers, but Nrp1/Nrp2 heterodimers also occur.

Nrp1 (also denoted CD304 or BDCA-4) was first identified as a receptor for the class 3 semaphorins (SEMA3) such as Sema3A, which are involved in axonal guidance in embryonic development. In this function, Nrp1 acts as a coreceptor for SEMA3 family members and promotes their interaction with plexins. Subsequently, the Nrp proteins were identified as coreceptors for several members of the vascular endothelial growth factor (VEGF) family. Nrp1 was found to interact with VEGF-A165 (and other VEGFs) and the receptor tyrosine kinase (RTK) VEGFR2, and to enhance signaling through this pathway and promote angiogenesis. Heparin markedly increases the affinity of VEGF for Nrp1, and appears to contribute to the formation of a complex incorporating VEGF, Nrp1 and VEGFR2. Nrp2 has different (but overlapping) binding preferences for VEGF family members, and is a coreceptor for VEGFR3 that is involved in lymphatic endothelial cell function.

The relationship between semphorins, plexins, and neuropilins is reviewed in Yamamoto, et al, International Immunology, 20(3):413-420 (2008). Semaphorins are a family of secreted and transmembrane proteins characterized by a conserved amino terminal ‘Sema domain’. Although they were originally identified as axon guidance factors during neuronal development, semaphorins have also been shown to have diverse and important physiological and pathological roles in cardiovascular development, tumor progression and immune regulation. A number of studies with gene-targeted mice have shown that some of membrane-bound semaphorins such as Sema4A, Sema4D, Sema6D and Sema7A are critically involved in immune regulation. In addition, it was recently reported that a secreted semaphorin, Sema3A, is involved in T cell regulation.

In the nervous system, neuropilin and plexin molecules serve as the major semaphorin receptors. In particular, plexins are critical for the transduction of semaphorins signals. Plexins can be divided structurally into four classes: plexin-A1-A4, plexin-B1-B3, plexin-C and plexin-D. Plexin-A class molecules not only form a receptor complex with neuropilins for secreted class III semaphorins but also binds directly to transmembrane class VI semaphorins in a neuropilin-independent manner. Plexin-B1 directly binds to a class IV semaphorin, Sema4D. Plexin-C1 has been reported to interact with the class VII semaphorin, Sema7A. Plexin-D1 has been shown to bind class III semaphorins in both a neuropilin-independent and -dependent manner. In the immune system, Plexin-A1 has been reported to be critically involved in dendritic cell (DC) functions.

The Examples below show that B7-H4-Ig binds to Nrp-1/Sema3a, PlexinA4/Sema3a, and Nrp-1/PlexinA4/Sema3a complexes. The immunoregulatory effects of B7-H4-Ig (decreased proliferation and IFN-γ production and enhanced IL-10 production) were not seen in cells derived from the mice on a C57Bl/6 background in which Nrp-1^{-/-} is conditionally deleted within FoxP3 expressing cells, i.e., Treg cells, with the exception that IL-17 production was decreased in cells from both wildtype and Nrp-1^{-/-} mice in the presence of B7-H4-Ig. B7-H4-Ig treatment decreased the level of secreted GM-CSF, IFN-γ, and IL-17 production and slightly increased the level of IL-10 production by T cells isolated from mice on a NOD background. However, in cultures containing cells isolated from mice on a NOD background in which Nrp-1^{-/-} is conditionally deleted within FoxP3 expressing cells, B7-H4-Ig treatment did not decrease the secreted levels of IFN-gamma, GM-CSF and IL-17 or appreciably affect the secreted level of IL-10.

The addition of either recombinant Nrp-1 or recombinant PlexinA4 alone does not alter the level of anti-CD3-induced T cell proliferation. In contrast, the treatment of the cultures with B7-H4-Ig decreased the level of anti-CD3-induced proliferation in a concentration-dependent manner, while the co-treatment of cultures with recombinant human Nrp-1 or recombinant human PlexinA4 partially blocked the immunosuppression function of B7-B4-Ig.

The treatment of Sema3a^{-/-} cultures with increasing concentrations of B7-H4-Ig does not alter the level of cytokines secreted, but the addition of exogenous recombinant human Sema3a recovers B7-H4-Ig function, i.e., a concentration-dependent decrease in the level of secreted GM-CSF, IFN-gamma, and IL-17, and increases the level of IL-10 secreted.

Furthermore, exogenous recombinant human Sema3a is able to decrease the level of 293 cell proliferation in a concentration-dependent manner. Similarly, the addition of soluble B7-H4-Ig was able to further decrease the level of 293 cell proliferation in a concentration-dependent manner.

Accordingly, molecules capable of binding to B7-H4 receptors that can modulate the binding between B7-H4 and B7-H4 receptors are disclosed and discussed in more detail below. These molecules can modulate the signal transduction that occurs as a consequence of B7-H4 binding to receptor, i.e., antagonists and agonists of B7-H4 receptors. Such modulation may result in attenuating the signal transduction or in completely blocking the ability of B7-H4 to bind to the receptor. In a further embodiment, such modulation may attenuate or completely neutralize the ability of B7-H4 to mediate signal transduction via interaction with the receptor.

In other embodiments the modulation of B7-H4 signal transduction mimics, enhances, or otherwise agonizes signal transduction via interaction with the receptor. For example, these molecules can enhance the interaction between B7-H4 and the receptor and facilitate receptor binding thereto, or bind to the receptor thereby mimicking the activity of the endogenous ligand. In a still further embodiment, the modulation alters the nature of the interaction of B7-H4 and the receptor so as to alter the nature of the elicited signal transduction. For example, such modulating molecules can, by binding to the receptor or a ligand of the receptor, alter the ability of the receptor to bind to other ligands and receptors and thereby alter the overall activity of the receptor.

Preferably, modulation of B7-H4 signal transduction will provide at least a 10% change in a measurable immune system activity, more preferably, at least a 50% change in such activity, or at least a 2-fold, 5-fold, 10-fold, or still more preferably, at least a 100-fold change in such activity.

As discussed in more detail below, agonists and antagonists of B7-H4 receptors can be used to modulate B7-H4 receptor-mediated signal transduction. Methods of using the receptor antagonists and agonists are therefore disclosed. Methods for modulating an immune response can include administering to a subject in need thereof an effective amount of a modulator of the receptor. Representative modulators include but are not limited to antibodies that agonize or antagonize signal transduction through B7-H4 receptors. Other preferred modulators include B7-H4 polypeptides, B7-H4 fusion proteins, receptor peptides and fusion proteins, and co-ligand peptides and fusion proteins. Antibodies that specifically bind an extracellular domain of B7-H4 or B7-H4 receptors are particularly useful for modulating immune responses.

A. Receptors for B7-H4

It has been discovered that neuropilins and plexins alone and in combination are receptors for B7-H4.

1. Neuropilins

Neuropilins are 120 to 130 kDa non-tyrosine kinase receptors. Multiple NRP-1 and NRP-2 isoforms exist, including soluble forms. The basic structure of neuropilins comprises five domains: three extracellular domains (a1a2, b1b2, and c), a transmembrane domain, and a short cytoplasmic domain. The a1a2 domain is a CUB domain (named for its identification in complement components C1r and C1s, Uegf, and bmp1), a domain commonly found in developmentally regulated proteins and which generally contains four cysteine residues that make two disulfide bridges. The neuropilin CUB domain shares homology with complement components C1r and C1s. The first two extracellular domains of NRP-1 (i.e., a1a2 and b1b2) bind ligand. Additionally, the structure-function studies using neuropilin mutants containing deletions within the “a” and “b” domains show that the CUB domains (a1a2 and b1b2) are required for semaphorin binding. The third extracellular domain is critical for homodimerization or heterodimerization (Ellis, L., Mol Cancer Ther, 5; 1099 (2006)).

a. NRP-1 Sequences

Nucleic acid and protein sequences for NRP-1, and variants and isoforms thereof are known in the art. See, for example, UniProtKB accession number 014786 (NRP1_HUMAN) which is specifically incorporated by reference herein in its entirety.

An exemplary amino acid sequence of NRP-1 is

MERGLPLLCA VLALVLAPAG AFRNDKCGDT IKIESPGYLT
SPGYPHSYHP SEKCEWLIQA PDPYQRIMIN FNPHFDLEDR
DCKYDYVEVF DGENENGHFR GKFCGKIAPP PVVSSGPFLF
IKFVSDYETH GAGFSIRYEI FKRGPECSQN YTTPSGVIKS
PGFPEKYPNS LECTYIVFVP KMSEIILEFE SFDLEPDSNP
PGGMFCRYDR LEIWDGFPDV GPHIGRYCGQ KTPGRIRSSS
GILSMVFYTD SAIAKEGFSA NYSVLQSSVS EDFKCMEALG
MESGEIHSDQ ITASSQYSTN WSAERSRLNY PENGWTPGED
SYREWIQVDL GLLRFVTAVG TQGAISKETK KKYYVKTYKI
DVSSNGEDWI TIKEGNKPVL FQGNTNPTDV VVAVFPKPLI
TRFVRIKPAT WETGISMRFE VYGCKITDYP CSGMLGMVSG
LISDSQITSS NQGDRNWMPE NIRLVTSRSG WALPPAPHSY
INEWLQIDLG EEKIVRGIII QGGKHRENKV FMRKFKIGYS
NNGSDWKMIM DDSKRKAKSF EGNNNYDTPE LRTFPALSTR
FIRIYPERAT HGGLGLRMEL LGCEVEAPTA GPTTPNGNLV
DECDDDQANC HSGTGDDFQL TGGTTVLATE KPTVIDSTIQ
SEFPTYGFNC EFGWGSHKTF CHWEHDNHVQ LKWSVLTSKT
GPIQDHTGDG NFIYSQADEN QKGKVARLVS PVVYSQNSAH
CMTFWYHMSG SHVGTLRVKL RYQKPEEYDQ LVWMAIGHQG
DHWKEGRVLL HKSLKLYQVI FEGEIGKGNL GGIAVDDISI
NNHISQEDCA KPADLDKKNP EIKIDETGST PGYEGEGEGD
KNISRKPGNV LKTLDPILIT IIAMSALGVL LGAVCGVVLY
CACWHNGMSE RNLSALENYN FELVDGVKLK KDKLNTQSTY
SEA

(SEQ ID NO:1, (NRP1_HUMAN), (“membrane bound form”)).

Another exemplary amino acid sequence for NRP-1 is

MERGLPLLCA VLALVLAPAG AFRNDKCGDT IKIESPGYLT
SPGYPHSYHP SEKCEWLIQA PDPYQRIMIN FNPHFDLEDR
DCKYDYVEVF DGENENGHFR GKFCGKIAPP PVVSSGPFLF
IKFVSDYETH GAGFSIRYEI FKRGPECSQN YTTPSGVIKS
PGFPEKYPNS LECTYIVFAP KMSEIILEFE SFDLEPDSNP
PGGMFCRYDR LEIWDGFPDV GPHIGRYCGQ KTPGRIRSSS
GILSMVFYTD SAIAKEGFSA NYSVLQSSVS EDFKCMEALG
MESGEIHSDQ ITASSQYSTN WSAERSRLNY PENGWTPGED
SYREWIQVDL GLLRFVTAVG TQGAISKETK KKYYVKTYKI
DVSSNGEDWI TIKEGNKPVL FQGNTNPTDV VVAVFPKPLI
TRFVRIKPAT WETGISMRFE VYGCKITDYP CSGMLGMVSG
LISDSQITSS NQGDRNWMPE NIRLVTSRSG WALPPAPHSY
INEWLQIDLG EEKIVRGIII QGGKHRENKV FMRKFKIGYS
NNGSDWKMIM DDSKRKAKSF EGNNNYDTPE LRTFPALSTR
FIRIYPERAT HGGLGLRMEL LGCEVEAPTA GPTTPNGNLV
DECDDDQANC HSGTGDDFQL TGGTTVLATE KPTVIDSTIQ
SEFPTYGFNC EFGWGSHKTF CHWEHDNHVQ LKWSVLTSKT
GPIQDHTGDG NFIYSQADEN QKGKVARLVS PVVYSQNSAH
CMTFWYHMSG SHVGTLRVKL RYQKPEEYHQ LVWMAIGHQG
DHWKEGRVLL HKSLKLYQVI FEGEIGKGNL GGIAVDDISI
NNHISQEDCA RSTPGYEGEG EGDKNISRKP GNVLKTLDPI
LITIIAMSAL GVLLGAVCGV VLYCACWHNG MSERNLSALE
NYNFELVDGV KLKKDKLNTQ STYSEA

(SEQ ID NO:37) (Genbank Accession No. AAP80144.1).

Another exemplary amino acid sequence for NRP-1 is

MERGLPLLCA VLALVLAPAG AFRNDKCGDT IKIESPGYLT
SPGYPHSYHP SEKCEWLIQA PDPYQRIMIN FNPHFDLEDR
DCKYDYVEVF DGENENGHFR GKFCGKIAPP PVVSSGPFLF
IKFVSDYETH GAGFSIRYEI FKRGPECSQN YTTPSGVIKS
PGFPEKYPNS LECTYIVFAP KMSEIILEFE SFDLEPDSNP
PGGMFCRYDR LEIWDGFPDV GPHIGRYCGQ KTPGRIRSSS
GILSMVFYTD SAIAKEGFSA NYSVLQSSVS EDFKCMEALG
MESGEIHSDQ ITASSQYSTN WSAERSRLNY PENGWTPGED
SYREWIQVDL GLLRFVTAVG TQGAISKETK KKYYVKTYKI
DVSSNGEDWI TIKEGNKPVL FQGNTNPTDV VVAVFPKPLI
TRFVRIKPAT WETGISMRFE VYGCKITDYP CSGMLGMVSG
LISDSQITSS NQGDRNWMPE NIRLVTSRSG WALPPAPHSY
INEWLQIDLG EEKIVRGIII QGGKHRENKV FMRKFKIGYS
NNGSDWKMIM DDSKRKAKSF EGNNNYDTPE LRTFPALSTR
FIRIYPERAT HGGLGLRMEL LGCEVEAPTA GPTTPNGNLV
DECDDDQANC HSGTGDDFQL TGGTTVLATE KPTVIDSTIQ
SGIK

(SEQ ID NO:63, (NCBI Reference Sequence: NP_001019799.1, neuropilin-1 isoform b precursor [Homo sapiens])).

Isoform 1 (SEQ ID NO:1) is considered a canonical NRP-1 sequence. Predicted protein domains, other isoforms, bindings sites, amino acid modifications, and known variants can be defined with reference to the canonical sequence (SEQ ID NO:1), as discussed in UniProtKB accession number 014786 (NRP1_HUMAN), excerpts of which are reproduced in the Tables below.

TABLE 1
Predicted Protein Domains

| | Position(s) | Length | Description |
|---|---|---|---|
| Signal peptide | 1-21 | 21 | |
| Chain | 22-923 | 902 | Neuropilin-1 |
| Topological domain | 22-856 | 835 | Extracellular Potential |
| Transmembrane | 857-879 | 23 | Helical; Potential |
| Topological domain | 880-923 | 44 | Cytoplasmic Potential |
| Domain | 27-141 | 115 | CUB 1 |
| Domain | 147-265 | 119 | CUB 2 |
| Domain | 275-424 | 150 | F5/8 type C 1 |
| Domain | 431-583 | 153 | F5/8 type C 2 |
| Domain | 645-811 | 167 | MAM |

TABLE 2
Metal Binding Sites

| | Position(s) | Length | Description |
|---|---|---|---|
| Metal binding | 195 | 1 | Calcium |
| Metal binding | 209 | 1 | Calcium |
| Metal binding | 250 | 1 | Calcium |

TABLE 3
Amino Acid Modifications

| | Position(s) | Length | Description |
|---|---|---|---|
| Glycosylation | 150 | 1 | N-linked |
| | | | (GlcNAc . . .) |
| Glycosylation | 261 | 1 | N-linked |
| | | | (GlcNAc . . .) |
| Glycosylation | 300 | 1 | N-linked |
| | | | (GlcNAc . . .) Potential |
| Glycosylation | 522 | 1 | N-linked (GlcNAc . . .) |
| Glycosylation | 612 | 1 | O-linked (Xyl . . .) |
| | | | (chondroitin sulfate); |
| | | | alternate |
| Glycosylation | 612 | 1 | O-linked (Xyl . . .) |
| | | | (heparan sulfate); |
| | | | alternate |
| Glycosylation | 842 | 1 | N-linked |
| | | | (GlcNAc . . .) Potential |
| Disulfide bond | 27 [symbol] 54 | | Probable |
| Disulfide bond | 82 [symbol] 104 | | Probable |
| Disulfide bond | 147 [symbol] 173 | | |
| Disulfide bond | 206 [symbol] 228 | | |
| Disulfide bond | 275 [symbol] 424 | | |
| Disulfide bond | 431 [symbol] 583 | | |

TABLE 4
Variants

| | Position(s) | Length | Description |
|---|---|---|---|
| Alternative | 587-621 | 35 | Missing in |
| sequence | | | isoform 3. |
| Alternative | 642-644 | 3 | EFP → GIK in |
| sequence | | | isoform 2 and |
| | | | isoform 3. |
| Alternative | 645-923 | 279 | Missing in |
| sequence | | | isoform 2 and |
| | | | isoform 3. |
| Natural variant | 179 | 1 | V → A. |
| | | | Corresponds to |
| | | | variant rs7079053 |
| | | | [dbSNP \| Ensembl]. |
| Natural variant | 561 | 1 | F → L. |
| | | | Corresponds to |
| | | | variant rs2228637 |
| | | | [dbSNP \| Ensembl]. |
| Natural variant | 733 | 1 | V → I. |
| | | | Corresponds to |
| | | | variant rs2228638 |
| | | | [dbSNP \| Ensembl]. |
| Sequence conflict | 26 | 1 | K → E in AAC51759. |
| Sequence conflict | 219 | 1 | D → G in CAD91133. |
| Sequence conflict | 749 | 1 | D → H in AAC12921. |
| Sequence conflict | 855 | 1 | D → E in AAC51759. |

Common alternative isoforms include isoform 2 (identifier: O14786-2), also known as: Soluble; SNRP1; which differs from the canonical sequence as follows: 642-644: EFP→GIK, 645-923: missing; isoform 3 (identifier: O14786-3) which differs from the canonical sequence as follows: 587-621: missing, 642-644: EFP→GIK, 645-923: missing.

b. NRP-2 Sequences

Nucleic acid and protein sequences for NRP-1, and variants and isoforms thereof are known in the art. See, for example, UniProtKB accession number O14786 (NRP1_HUMAN) which is specifically incorporated by reference herein in its entirety.

An exemplary amino acid sequence of NRP-2 is

MDMFPLTWVF LALYFSRHQV RGQPDPPCGG RLNSKDAGYI
TSPGYPQDYP SHQNCEWIVY APEPNQKIVL NFNPHFEIEK
HDCKYDFIEI RDGDSESADL LGKHCGNIAP PTIISSGSML
YIRFTSDYAR QGAGFSLRYE IFKTGSEDCS KNFTSPNGTI
ESPGFPEKYP HNLDCTFTIL AKPKMEIILQ FLIFDLEHDP
LQVGEGDCKY DWLDIWDGIP HVGPLIGKYC GTKTPSELRS
STGILSLTFH TDMAVAKDGF SARYYLVHQE PLENFQCNVP
LGMESGRIAN EQISASSTYS DGRWTPQQSR LHGDDNGWTP
NLDSNKEYLQ VDLRFLTMLT AIATQGAISR EIQNGYYVKS
YKLEVSTNGE DWMVYRHGKN HKVFQANNDA TEVVLNKLHA
PLLTRFVRIR PQTWHSGIAL RLELFGCRVT DAPCSNMLGM
LSGLIADSQI SASSTQEYLW SPSAARLVSS RSGWFPRIPQ
AQPGEEWLQV DLGTPKTVKG VIIQGARGGD SITAVEARAF
VRKFKVSYSL NGKDWEYIQD PRTQQPKLFE GNMHYDTPDI
RRFDPIPAQY VRVYPERWSP AGIGMRLEVL GCDWIDSKPT
VETLGPTVKS EETTTPYPTE EEATECGENC SFEDDKDLQL
PSGFNCNFDF LEEPCGWMYD HAKWLRTTWA SSSSPNDRTF
PDDRNFLRLQ SDSQREGQYA RLISPPVHLP RSPVCMEFQY
QATGGRGVAL QVVREASQES KLLWVIREDQ GGEWKHGRII
LPSYDMEYQI VFEGVIGKGR SGEIAIDDIR ISTDVPLENC
MEPISAFAGE NFKVDIPEIH EREGYEDEID DEYEVDWSNS
SSATSGSGAP STDKEKSWLY TLDPILITII AMSSLGVLLG
ATCAGLLLYC TCSYSGLSSR SCTTLENYNF ELYDGLKHKV
KMNHQKCCSE A

### (SEQ ID NO:38) (O60462 (NRP2_HUMAN)).

An alternative amino acid sequence for human neuropilin 2 is

MDMFPLTWVF LALYFSRHQV RGQPDPPCGG RLNSKDAGYI
TSPGYPQDYP SHQNCEWIVY APEPNQKIVL NFNPHFEIEK
HDCKYDFIEI RDGDSESADL LGKHCGNIAP PTIISSGSML
YIKFTSDYAR QGAGFSLRYE IFKTGSEDCS KNFTSPNGTI
ESPGFPEKYP HNLDCTFTIL AKPKMEIILQ FLIFDLEHDP
LQVGEGDCKY DWLDIWDGIP HVGPLIGKYC GTKTPSELRS
STGILSLTFH TDMAVAKDGF SARYYLVHQE PLENFQCNVP
LGMESGRIAN EQISASSTYS DGRWTPQQSR LHGDDNGWTP
NLDSNKEYLQ VDLRFLTMLT AIATQGAISR ETQNGYYVKS
YKLEVSTNGE DWMVYRHGKN HKVFQANNDA TEVVLNKLHA
PLLTRFVRIR PQTWHSGIAL RLELFGCRVT DAPCSNMLGM
LSGLIADSQI SASSTQEYLW SPSAARLVSS RSGWFPRIPQ
AQPGEEWLQV DLGTPKTVKG VIIQGARGGD SITAVEARAF
VRKFKVSYSL NGKDWEYIQD PRTQQPKLFE GNMHYDTPDI
RRFDPIPAQY VRVYPERWSP AGIGMRLEVL GCDWTDSKPT
VETLGPTVKS EETTTPYPTE EEATECGENC SFEDDKDLQL
PSGFNCNFDF LEEPCGWMYD HAKWLRTTWA SSSSPNDRTF
PDDRNFLRLQ SDSQREGQYA RLISPPVHLP RSPVCMEFQY
QATGGRGVAL QVVREASQES KLLWVIREDQ GGEWKHGRII
LPSYDMEYQI VFEGVIGKGR SGEIAIDDIR ISTDVPLENC
MEPISAFAGG TLLPGTEPTV DTVPMQPIPA YWYYVMAAGG
AVLVLVSVAL ALVLHYHRFR YAAKKTDHSI TYKTSHYTNG
APLAVEPTLT IKLEQDRGSH C

### (SEQ ID NO:39) (NCBI Reference Sequence: NP_957719.1 (Isoform 5)).

Isoform A22 (SEQ ID NO:38) is considered a canonical NRP-2 sequence. Predicted protein domains, other isoforms, bindings sites, amino acid modifications, and known variants can be defined with reference to the canonical sequence (SEQ ID NO:38), as discussed in UniProtKB accession number O60462 (NRP2_HUMAN), excerpts of which are reproduced in the Tables below.

TABLE 5
Predicted Protein Domains

| | Position(s) | Length | Description |
|---|---|---|---|
| Signal peptide | 1-20 | 20 | Or 22 Potential |
| Chain | 21-931 | 911 | Neuropilin-2 |
| Topological domain | 21-864 | 844 | Extracellular Potential |
| Transmembrane | 865-889 | 25 | Helical; Potential |
| Topological domain | 890-931 | 42 | Cytoplasmic Potential |
| Domain | 28-142 | 115 | CUB 1 |
| Domain | 149-267 | 119 | CUB 2 |
| Domain | 277-427 | 151 | F5/8 type C 1 |
| Domain | 434-592 | 159 | F5/8 type C 2 |
| Domain | 642-802 | 161 | MAM |
| Compositional bias | 671-674 | 4 | Poly-Ser |

TABLE 6
Metal Binding Sites

| | Position(s) | Length | Description |
|---|---|---|---|
| Metal binding | 197 | 1 | Calcium |
| Metal binding | 211 | 1 | Calcium |
| Metal binding | 252 | 1 | Calcium |

TABLE 7
Amino Acid Modifications

| | Position(s) | Length | Description |
|---|---|---|---|
| Glycosylation | 152 | 1 | N-linked (GlcNAc . . .) |
| Glycosylation | 157 | 1 | N-linked (GlcNAc . . .) |
| Glycosylation | 629 | 1 | N-linked |
| | | | (GlcNAc . . .) Potential |
| Glycosylation | 839 | 1 | N-linked |
| | | | (GlcNAc . . .) Potential |
| Disulfide bond | 28 [symbol] 55 | | |
| Disulfide bond | 83 [symbol] 105 | | |
| Disulfide bond | 149 [symbol] 175 | | |
| Disulfide bond | 208 [symbol] 230 | | |
| Disulfide bond | 277 [symbol] 427 | | |
| Disulfide bond | 434 [symbol] 592 | | |

TABLE 8
Variants

| | Position(s) | Length | Description |
|---|---|---|---|
| Alternative sequence | 548-555 | 8 | LFEGNMHY (SEQ ID NO: 81) → |
| | | | VGCSWRPL (SEQ ID NO: 82) in |
| | | | isoform s9. |
| Alternative sequence | 556-931 | 376 | Missing in isoform s9. |
| Alternative sequence | 809-830 | 22 | Missing in isoform A0. |
| Alternative sequence | 809-813 | 5 | Missing in isoform A17 and |
| | | | isoform B0. |
| Alternative sequence | 814-931 | 118 | VDIPE . . . CCSEA (SEQ ID NO: 75) |
| | | | → GGTLLPGTEPTVDTVPMQPI |
| | | | PAYWYYVMAAGGAVLVLVSV |
| | | | ALALVLHYHRFRYAAKKTDH |
| | | | SITYKTSHYTNGAPLAVEPT |
| | | | LTIKLEQDRGSHC (SEQ ID NO: 76) |
| | | | in isoform B0 and isoform B5. |
| Natural variant | 123 | 1 | R → K. |
| | | | Corresponds to variant rs849541 |
| | | | [dbSNP\|Ensembl]. |
| Natural variant | 334 | 1 | R → C Rare variant; may act as a |
| | | | phenotype modifier in EIEE13 |
| | | | patients carrying SCN8A mutations. |
| | | | Corresponds to variant rs114144673 |
| | | | [dbSNP\|Ensembl]. |
| Natural variant | 428 | 1 | R → W Rare variant; may act as a |
| | | | phenotype modifier in EIEE13 |
| | | | patients carrying SCN8A mutations. |
| | | | Corresponds to variant rs139711818 |
| | | | [dbSNP\|Ensembl]. |
| Natural variant | 602 | 1 | E → K. |
| | | | Corresponds to variant rs1128169 |
| | | | [dbSNP\|Ensembl]. |

Common alternative splice forms include isoform A0 (identifier: O60462-2) which differs from the canonical sequence as follows: 809-830 missing; isoform A17 (identifier: O60462-3), which differs from the canonical sequence as follows: 809-813 missing; isoform B0 (identifier: O60462-4) which differs from the canonical sequence as follows: 809-813 missing, 814-931: VDIPEIHERE . . . MNHQKCCSEA (SEQ ID NO:77)→GGTLLPGTEP . . . KLEQDRGSHC (SEQ ID NO:78); isoform B5 (identifier: O60462-5), which differs from the canonical sequence as follows: 814-931: VDIPEIHERE . . . MNHQKCCSEA (SEQ ID NO:79)→GGTLLPGTEP . . . KLEQDRGSHC (SEQ ID NO:80); and isoform s9 (identifier: 060462-6) which differs from the canonical sequence as follows: 548-555: LFEGNMHY (SEQ ID NO:81)→VGCSWRPL (SEQ ID NO:82), 556-931: missing.

3. Plexins

It has been discovered that Plexins, particularly Plexin4A is a receptor for B7-H4, particularly when it is a co-receptor complex in combination with a neruopilin such as NRP-1. Plexin4A was previously identified as a co-receptor for class 3 semaphorins, which is needed for semaphorin signaling that leads to remodeling of the cytoskeleton. Class 3 semaphorins bind to a complex composed of a neuropilin and a plexin. The plexin modulates the affinity of the complex for specific semaphorins, and its cytoplasmic domain is needed for the activation of down-stream signaling events in the cytoplasm. The data presented in the Examples below indicate that plexin can also increase the binding of B7-H4, and Ig fusion proteins thereof, to neuropilin 1. The association of a Semaphorin, such as Sema3a or Sema6c may further increase the binding of B7-H4 to the receptor complex.

Nucleic acid and protein sequences for Plexins such as Plexin4A, and variants and isoforms thereof are known in the art. See, for example, UniProtKB accession number Q9HCM2 (PLXA4_HUMAN) which is specifically incorporated by reference herein in its entirety.

An exemplary amino acid sequence for Plexin4A is

MKAMPWNWTC LLSHLLMVGM GSSTLLTRQP APLSQKQRSF
VTFRGEPAEG FNHLVVDERT GHIYLGAVNR IYKLSSDLKV
LVTHETGPDE DNPKCYPPRI VQTCNEPLTT TNNVNKMLLI
DYKENRLIAC GSLYQGICKL LRLEDLFKLG EPYHKKEHYL
SGVNESGSVF GVIVSYSNLD DKLFIATAVD GKPEYFPTIS
SRKLTKNSEA DGMFAYVFHD EFVASMIKIP SDTFTIIPDF
DIYYVYGFSS GNFVYFLTLQ PEMVSPPGST TKEQVYTSKL
VRLCKEDTAF NSYVEVPIGC ERSGVEYRLL QAAYLSKAGA
VLGRTLGVHP DDDLLFIVFS KGQKRKMKSL DESALCIFIL
KQINDRIKER LQSCYRGEGT LDLAWLKVKD IPCSSALLTI
DDNFCGLDMN APLGVSDMVR GIPVFTEDRD RMTSVIAYVY
KNHSLAFVGT KSGKLKKIRV DGPRGNALQY ETVQVVDPGP
VLRDMAFSKD HEQLYIMSER QLTRVPVESC GQYQSCGECL
GSGDPHCGWC VLHNTCTRKE RCERSKEPRR FASEMKQCVR
LTVHPNNISV SQYNVLLVLE TYNVPELSAG VNCTFEDLSE
MDGLVVGNQI QCYSPAAKEV PRIITENGDH HVVQLQLKSK
ETGMTFASTS FVFYNCSVHN SCLSCVESPY RCHWCKYRHV
CTHDPKTCSF QEGRVKLPED CPQLLRVDKI LVPVEVIKPI
TLKAKNLPQP QSGQRGYECI LNIQGSEQRV PALRFNSSSV
QCQNISYSYE GMEINNLPVE LTVVWNGHFN IDNPAQNKVH
LYKCGAMRES CGLCLKADPD FACGWCQGPG QCTLRQHCPA
QESQWLELSG AKSKCINPRI TEIIPVTGPR EGGTKVTIRG
ENLGLEFRDI ASHVKVAGVE CSPLVDGYIP AEQIVCEMGE
AKPSQHAGFV EICVAVCRPE FMARSSQLYY FMTLTLSDLK
PSRGPMSGGT QVTITGTNLN AGSNVVVMFG KQPCLFHRRS
PSYIVCNTTS SDEVLEMKVS VQVDRAKIHQ DLVFQYVEDP
TIVRIEPEWS IVSGNTPIAV WGTHLDLIQN PQIRAKHGGK
EHINICEVLN ATEMTCQAPA LALGPDHQSD LTERPEEFGF
ILDNVQSLLI LNKTNFTYYP NPVFEAFGPS GILELKPGTP
IILKGKNLIP PVAGGNVKLN YTVLVGEKPC TVTVSDVQLL
CESPNLIGRH KVMARVGGME YSPGMVYIAP DSPLSLPAIV
SIAVAGGLLI IFIVAVLIAY KRKSRESDLT LKRLQMQMDN
LESRVALECK EAFAELQTDI HELISDLDGA GIPFLDYRTY
TMRVLFPGIE DHPVLRDLEV PGYRQERVEK GLKLFAQLIN
NKVFLLSFIR TLESQRSFSM RDRGNVASLI MTVLQSKLEY
ATDVLKQLLA DLIDKNLESK NHPKLLLRRI ESVAEKMLTN
WFTFLLYKFL KECAGEPLFS LFCAIKQQME KGPIDAITGE
ARYSLSEDKL IRQQIDYKTL VLSCVSPDNA NSPEVPVKIL
NCDTITQVKE KILDAIFKNV PCSHRPKAAD MDLEWRQGSG
ARMILQDEDI TTKIENDWKR LNTLAHYQVP DGSVVALVSK
QVTAYNAVNN STVSRTSASK YENMIRYTGS PDSLRSRTPM
ITPDLESGVK MWHLVKNHEH GDQKEGDRGS KMVSEIYLTR
LLATKGTLQK FVDDLFETIF SIAHRGSALP LAIKYMFDFL
DEQADKHGIH DPHVRHTWKS NCLPLRFWVN MIKNPQFVFD
IHKNSITDAC LSVVAQTFMD SCSTSEHRLG KDSPSNKLLY
AKDIPSYKNW VERYYSDIGK MPAISDQDMN AYLAEQSRMH
MNEFNTMSAL SEIFSYVGKY SEEILGPLDH DDQCGKQKLA
YKLEQVITLM SLDS

### (SEQ ID NO:2, Q9HCM2 (PLXA4_HUMAN)).

Isoform 1 (SEQ ID NO:2) is considered a canonical Plexin4A sequence. Predicted protein domains, other isoforms, bindings sites, amino acid modifications, and known variants can be defined with reference to the canonical sequence (SEQ ID NO:2), as discussed in UniProtKB accession number Q9HCM2 (PLXA4_HUMAN), excerpts of which are reproduced in the Tables below.

TABLE 9
Predicted Protein Domains

| | Position(s) | Length | Description |
|---|---|---|---|
| Signal peptide | 1-23 | 23 | Potential |
| Chain | 24-1894 | 1871 | Plexin-A4 |
| Topological domain | 24-1237 | 1214 | Extracellular Potential |
| Transmembrane | 1238-1258 | 21 | Helical; Potential |
| Topological domain | 1259-1894 | 636 | Cytoplasmic Potential |
| Domain | 24-507 | 484 | Sema |
| Domain | 509-559 | 51 | PSI 1 |
| Domain | 655-702 | 48 | PSI 2 |
| Domain | 803-856 | 54 | PSI 3 |
| Domain | 858-952 | 95 | IPT/TIG 1 |
| Domain | 954-1037 | 84 | IPT/TIG 2 |
| Domain | 1040-1139 | 100 | IPT/TIG 3 |
| Domain | 1142-1230 | 89 | IPT/TIG 4 |

TABLE 10
Amino Acid Modifications

| | Position(s) | Length | Description |
|---|---|---|---|
| Modified residue | 1350 | 1 | N6-acetyllysine |
| Glycosylation | 655 | 1 | N-linked |
| | | | (GlcNAc . . .) Potential |
| Glycosylation | 1007 | 1 | N-linked |
| | | | (GlcNAc . . .) Potential |
| Glycosylation | 1132 | 1 | N-linked |
| | | | (GlcNAc . . .) Potential |
| Glycosylation | 1180 | 1 | N-linked |
| | | | (GlcNAc . . .) Potential |
| Disulfide bond | 95 [symbol] 104 | | By similarity |
| Disulfide bond | 130 [symbol] 138 | | By similarity |
| Disulfide bond | 284 [symbol] 405 | | By similarity |
| Disulfide bond | 300 [symbol] 356 | | By similarity |
| Disulfide bond | 374 [symbol] 393 | | By similarity |
| Disulfide bond | 510 [symbol] 527 | | By similarity |
| Disulfide bond | 516 [symbol] 558 | | By similarity |
| Disulfide bond | 519 [symbol] 536 | | By similarity |
| Disulfide bond | 530 [symbol] 542 | | By similarity |
| Disulfide bond | 593 [symbol] 612 | | By similarity |

TABLE 11
Variants

| | Position(s) | Length | Description |
|---|---|---|---|
| Alternative | 1-1550 | 1550 | Missing in isoform 4. |
| sequence | | | |
| Alternative | 458-522 | 65 | IRVDG . . . ECLGS (SEQ ID |
| sequence | | | NO: 83) → |
| | | | SFGTGPQGGITQEWIGVEGD |
| | | | PPGANIASQEQMLCVYLQCS |
| | | | SHKAISDQRVQPLLCCFLNV |
| | | | PGNSS (SEQ ID NO: 84) in |
| | | | isoform 3. |
| Alternative | 458-492 | 35 | IRVDG . . . SKDHE (SEQ ID |
| sequence | | | NO: 85) → |
| | | | MPGTSLCPTLELQTGPRSHR |
| | | | ATVTLELLFSSCSSN (SEQ ID |
| | | | NO: 86) in isoform 2. |
| Alternative | 493-1894 | 1402 | Missing in isoform 2. |
| sequence | | | |
| Alternative | 523-1894 | 1372 | Missing in isoform 3. |
| sequence | | | |
| Sequence conflict | 1750 | 1 | N → D in CAD39161. |
| Sequence conflict | 1804 | 1 | I → V in CAD39161. |
| Sequence conflict | 458 | 1 | M → V in BAC85615. |
| Sequence conflict | 458 | 1 | M → V in AAQ89209. |
| Sequence conflict | 475 | 1 | E → A in AAH28744. |

Other common isoforms include isoform 2 (identifier: Q9HCM2-2), which differs from the canonical sequence as follows: 458-492: IRVDGPRGNALQYETVQVVDPGPVLRDMAFSKDHE (SEQ ID NO:87)→MPGTSLCPTLELQTGPRSHRATVTLELLFSSCSSN (SEQ ID NO:88), 493-1894: missing; isoform 3 (identifier: Q9HCM2-3), which differs from the canonical sequence as follows: 458-522: IRVDGPRGNA . . . YQSCGECLGS (SEQ ID NO:89)→SFGTGPQGGI . . . CFLNVPGNSS (SEQ ID NO:90), 523-1894: missing, and isoform 4 (identifier: Q9HCM2-4), which differs from the canonical sequence as follows: 1-1550: missing.

B. Co-Ligands of B7-H4 Receptors

It has been discovered that semaphorins can serve as a co-ligand with B7-H4 for B7-H4 receptors, for example, NRP-1/Plexin4A receptor complexes. Semaphorins are a class of secreted and membrane proteins that have been characterized based on their role as axonal growth cone guidance molecules. They can act as short-range inhibitory signals and signal through multimeric receptor complexes. As discussed above, a major class of proteins that act as semaphorin receptors are called plexins. Semphorins are known in the art, and include, for example, Sema6C and Sema 3A,

1. SEMA6C

Nucleic acid and protein sequences for Semaphorins such as SEMA6C, and variants and isoforms thereof are known in the art. See, for example, UniProtKB accession number Q9H3T2 (SEM6C_HUMAN) which is specifically incorporated by reference herein in its entirety.

An exemplary amino acid sequence for SEMA6C is

MPRAPHFMPL LLLLLLLSLP HTQAAFPQDP LPLLISDLQG
TSPLSWFRGL EDDAVAAELG LDFQRFLTLN RTLLVAARDH
VFSFDLQAEE EGEGLVPNKY LTWRSQDVEN CAVRGKLTDE
CYNYIRVLVP WDSQTLLACG TNSFSPVCRS YGIISLQQEG
EELSGQARCP FDATQSNVAI FAEGSLYSAT AADFQASDAV
VYRSLGPQPP LRSAKYDSKW LREPHFVQAL EHGDHVYFFF
REVSVEDARL GRVQFSRVAR VCKRDMGGSP RALDRHWISF
LKLRLNCSVP GDSTFYFDVL QALTGPVNLH GRSALFGVFT
TQTNSIPGSA VCAFYLDEIE RGFEGKFKEQ RSLDGAWTPV
SEDRVPSPRP GSCAGVGGAA LFSSSRDLPD DVLTFIKAHP
LLDPAVPPVT HQPLLTLTSR ALLTQVAVDG MAGPHSNITV
MFLGSNDGTV LKVLTPGGRS GGPEPILLEE IDAYSPARCS
GKRTAQTARR IIGLELDTEG HRLFVAFSGC IVYLPLSRCA
RHGACQRSCL ASQDPYCGWH SSRGCVDIRG SGGTDVDQAG
NQESMEHGDC QDGATGSQSG PGDSAYGVRR DLPPASASRS
VPIPLLLASV AAAFALGASV SGLLVSCACR RAHRRRGKDI
EIPGLPRPLS LRSLARLHGG GPEPPPPSKD GDAVQTPQLY
TTFLPPPEGV PPPELACLPT PESTPELPVK HLRAAGDPWE
WNQNRNNAKE GPGRSRGGHA AGGPAPRVLV RPPPPGCPGQ
AVEVTTLEEL LRYLHGPQPP RKGAEPPAPL TSRALPPEPA
PALLGGPSPR PHECASPLRL DVPPEGRCAS APARPALSAP
APRLGVGGGR RLPFSGHRAP PALLTRVPSG GPSRYSGGPG
KHLLYLGRPE GYRGRALKRV DVEKPQLSLK PPLVGPSSRQ
AVPNGGRFNF

### (SEQ ID NO:40, Q9H3T2 (SEM6C_HUMAN)).

Exemplary, alternative SEMA6C amino acid sequences are

MPRAPHFMPL LLLLLLLSLP HTQAAFPQDP LPLLISDLQG
TSPLSWFRGL EDDAVAAELG LDFQRFLTLN RTLLVAARDH
VFSFDLQAEE EGEGLVPNKY LTWRSQDVEN CAVRGKLTDE
CYNYIRVLVP WDSQTLLACG TNSFSPVCRS YGITSLQQEG
EELSGQARCP FDATQSNVAI FAEGSLYSAT AADFQASDAV
VYRSLGPQPP LRSAKYDSKW LREPHFVQAL EHGDHVYFFF
REVSVEDARL GRVQFSRVAR VCKRDMGGSP RALDRHWTSF
LKLRLNCSVP GDSTFYFDVL QALTGPVNLH GRSALFGVFT
TQTNSIPGSA VCAFYLDEIE RGFEGKFKEQ RSLDGAWTPV
SEDRVPSPRP GSCPAHSSPL GRSLQVLRGS REAPPVPGPA
RGLPGPRPEK GGRREAPVVP EASPRRALLP PGRPERRPFQ
LLKGAVHGLQ RGERPSPLGH ELDALQDVSP PPRPAPPAFP
TRRVSRGPFS PRVYLLTVFP PVLDRRVGGE KPVSSVSQHF
RGSWRTPTPR SLPAKLP

(SEQ ID NO:41, SEMA6C protein [Homo sapiens] GenBank: AAI14624.1), and

MPRAPHFMPL LLLLLLLSLP HTQAAFPQDP LPLLISDLQG
TSPLSWFRGL EDDAVAAELG LDFQRFLTLN RTLLVAARDH
VFSFDLQAEE EGEGLVPNKY LTWRSQDVEN CAVRGKLTDE
CYNYIRVLVP WDSQILLACG TNSFSPVCRS YGIISLQQEG
EELSGQARCP FDATQSNVAI FAEGSLYSAT AADFQASDAV
VYRSLGPQPP LRSAKYDSKW LREPHFVQAL EHGDHVYFFF
REVSVEDARL GRVQFSRVAR VCKRDMGGSP RALDRHWTSF
LKLRLNCSVP GDSTFYFDVL QALTGPVNLH GRSALFGVFT
TQTNSIPGSA VCASYLDEIE RGFEGKFKEQ RSLDGAWTPV
SEDRVPSPRP GSCAGVGGAA LFSSSRDLPS SGPPPARPSR
TAAVSTFKGS GPRPPAWGAP ESSRPRAGRS SGRFTAPSPR
TSSLPDSQSL PRPLFASGLF IDCLSPCPRQ KSGR

(SEQ ID NO:42, SEMA6C protein [Homo sapiens] GenBank: AAI14522.1),

Isoform 1 (SEQ ID NO:40) is considered a canonical SEMA6C sequence. Predicted protein domains, other isoforms, bindings sites, amino acid modifications, and known variants can be defined with reference to the canonical sequence (SEQ ID NO:40), as discussed in UniProtKB accession number Q9H3T2 (SEM6C_HUMAN), excerpts of which are reproduced in the Tables below.

TABLE 12
Predicted Protein Domains

| | Position(s) | Length | Description |
|---|---|---|---|
| Signal peptide | 1-24 | 24 | Potential |
| Chain | 25-930 | 906 | Semaphorin-6C |
| Topological domain | 25-604 | 580 | Extracellular Potential |
| Transmembrane | 605-625 | 21 | Helical; Potential |
| Topological domain | 626-930 | 305 | Cytoplasmic Potential |
| Domain | 30-516 | 487 | Sema |
| Compositional bias | 662-667 | 6 | Poly-Pro |
| Compositional bias | 752-755 | 4 | Poly-Pro |

TABLE 13
Amino Acid Modifications

| | Position(s) | Length | Description |
|---|---|---|---|
| Glycosylation | 70 | 1 | N-linked |
| | | | (GlcNAc . . .) Potential |
| Glycosylation | 286 | 1 | N-linked |
| | | | (GlcNAc . . .) Potential |
| Glycosylation | 437 | 1 | N-linked |
| | | | (GlcNAc . . .) Potential |
| Disulfide bond | 111 [symbol] 121 | | By similarity |
| Disulfide bond | 139 [symbol] 148 | | By similarity |
| Disulfide bond | 262 [symbol] 373 | | By similarity |
| Disulfide bond | 287 [symbol] 332 | | By similarity |
| Disulfide bond | 479 [symbol] 510 | | By similarity |
| Disulfide bond | 519 [symbol] 537 | | By similarity |
| Disulfide bond | 525 [symbol] 570 | | By similarity |
| Disulfide bond | 529 [symbol] 545 | | By similarity |

TABLE 14
Variants

| | Position(s) | Length | Description |
|---|---|---|---|
| Alternative sequence | 184-223 | 40 | Missing in isoform 2. |
| Alternative sequence | 586 | 1 | Y → |
| | | | YVLPGPGPSPGTPSPPSDAH |
| | | | PRPQSSTLGVHTR (SEQ ID |
| | | | NO: 91) in isoform 2 and |
| | | | isoform 3. |
| Natural variant | 455 | 1 | T → P. |
| | | | Corresponds to variant |
| | | | rs4971007 |
| | | | [dbSNP\|Ensembl]. |

Other common isoforms include isoform 2 (identifier: Q9H3T2-2), also known as: Short 2; which differs from the canonical sequence as follows: 184-223: missing, 586-586: Y→YVLPGPGPSPGTPSPPSDAHPRPQSSTLGVHTR (SEQ ID NO:92); and isoform 3 (identifier: Q9H3T2-3), also known as: Long, which differs from the canonical sequence as follows: 586-586: Y→YVLPGPGPSPGTPSPPSDAHPRPQSSTLGVHTR (SEQ ID NO:93).

2. SEMA3A

Nucleic acid and protein sequences for Semaphorins such as SEMA3A, and variants and isoforms thereof are known in the art. See, for example, UniProtKB accession number Q14563 (SEM3A_HUMAN) which is specifically incorporated by reference herein in its entirety.

An exemplary amino acid sequence for SEMA3A is

MGWLTRIVCL FWGVLLTARA NYQNGKNNVP RLKLSYKEML
ESNNVITFNG LANSSSYHTF LLDEERSRLY VGAKDHIFSF
DLVNIKDFQK IVWPVSYTRR DECKWAGKDI LKECANFIKV
LKAYNQTHLY ACGTGAFHPI CTYIEIGHHP EDNIFKLENS
HFENGRGKSP YDPKLLTASL LIDGELYSGT AADFMGRDFA
IFRTLGHHHP IRTEQHDSRW LNDPKFISAH LISESDNPED
DKVYFFFREN AIDGEHSGKA THARIGQICK NDFGGHRSLV
NKWTTFLKAR LICSVPGPNG IDTHFDELQD VFLMNFKDPK
NPVVYGVFTT SSNIFKGSAV CMYSMSDVRR VFLGPYAHRD
GPNYQWVPYQ GRVPYPRPGT CPSKTFGGFD STKDLPDDVI
TFARSHPAMY NPVFPMNNRP IVIKTDVNYQ FTQIVVDRVD
AEDGQYDVMF IGTDVGTVLK VVSIPKETWY DLEEVLLEEM
TVFREPTAIS AMELSTKQQQ LYIGSTAGVA QLPLHRCDIY
GKACAECCLA RDPYCAWDGS ACSRYFPTAK RRTRRQDIRN
GDPLTHCSDL HHDNHHGHSP EERIIYGVEN SSTFLECSPK
SQRALVYWQF QRRNEERKEE IRVDDHIIRT DQGLLLRSLQ
QKDSGNYLCH AVEHGFIQTL LKVTLEVIDT EHLEELLHKD
DDGDGSKTKE MSNSMTPSQK VWYRDFMQLI NHPNLNTMDE
FCEQVWKRDR KQRRQRPGHT PGNSNKWKHL QENKKGRNRR
THEFERAPRS V

### (SEQ ID NO:43, Q14563 (SEM3A_HUMAN)).

SEQ ID NO:43 without the signal peptide is

NYQNGKNNVP RLKLSYKEML ESNNVITFNG LANSSSYHTF
LLDEERSRLY VGAKDHIFSF DLVNIKDFQK IVWPVSYTRR
DECKWAGKDI LKECANFIKV LKAYNQTHLY ACGTGAFHPI
CTYIEIGHHP EDNIFKLENS HFENGRGKSP YDPKLLTASL
LIDGELYSGT AADFMGRDFA IFRTLGHHHP IRTEQHDSRW
LNDPKFISAH LISESDNPED DKVYFFFREN AIDGEHSGKA
THARIGQICK NDFGGHRSLV NKWTTFLKAR LICSVPGPNG
IDTHFDELQD VFLMNFKDPK NPVVYGVFTT SSNIFKGSAV
CMYSMSDVRR VFLGPYAHRD GPNYQWVPYQ GRVPYPRPGT
CPSKTFGGFD STKDLPDDVI TFARSHPAMY NPVFPMNNRP
IVIKTDVNYQ FTQIVVDRVD AEDGQYDVMF IGTDVGTVLK
VVSIPKETWY DLEEVLLEEM TVFREPTAIS AMELSTKQQQ
LYIGSTAGVA QLPLHRCDIY GKACAECCLA RDPYCAWDGS
ACSRYFPTAK RRTRRQDIRN GDPLTHCSDL HHDNHHGHSP
EERIIYGVEN SSTFLECSPK SQRALVYWQF QRRNEERKEE
IRVDDHIIRT DQGLLLRSLQ QKDSGNYLCH AVEHGFIQTL
LKVTLEVIDT EHLEELLHKD DDGDGSKTKE MSNSMTPSQK
VWYRDFMQLI NHPNLNTMDE FCEQVWKRDR KQRRQRPGHT
PGNSNKWKHL QENKKGRNRR THEFERAPRS V

### (SEQ ID NO:62).

Isoform 1 (SEQ ID NO:43) is considered a canonical SEMA3A sequence. Predicted protein domains, other isoforms, bindings sites, amino acid modifications, and known variants can be defined with reference to the canonical sequence (SEQ ID NO:43), as discussed in UniProtKB accession number Q9H3T2 (SEM3A_HUMAN), excerpts of which are reproduced in the Tables below.

TABLE 15
Predicted Protein Domains

| | Position(s) | Length | Description |
|---|---|---|---|
| Signal peptide | 1-20 | 20 | Potential |
| Chain | 21-771 | 751 | Semaphorin-3A |
| Domain | 31-514 | 484 | Sema |
| Domain | 580-664 | 85 | Ig-like C2-type |
| Compositional bias | 727-769 | 43 | Arg/Lys-rich (basic) |

TABLE 16
Amino Acid Modifications

| | Position(s) | Length | Description |
|---|---|---|---|
| Glycosylation | 53 | 1 | N-linked |
| | | | (GlcNAc . . .) Potential |
| Glycosylation | 125 | 1 | N-linked |
| | | | (GlcNAc . . .) Potential |
| Glycosylation | 590 | 1 | N-linked |
| | | | (GlcNAc . . .) Potential |
| Disulfide bond | 103 [symbol] 114 | | By similarity |
| Disulfide bond | 132 [symbol] 141 | | By similarity |
| Disulfide bond | 269 [symbol] 381 | | By similarity |
| Disulfide bond | 293 [symbol] 341 | | By similarity |
| Disulfide bond | 517 [symbol] 535 | | By similarity |
| Disulfide bond | 649 [symbol] 722 | | By similarity |

TABLE 17
Variants

| | Position(s) | Length | Description |
|---|---|---|---|
| Natural variant | 66 | 1 | R → W in HH16; |
| | | | phenotype consistent |
| | | | with Kallmann |
| | | | syndrome. |
| | | | Corresponds to variant |
| | | | rs199979628 |
| | | | [dbSNP \| Ensembl]. |
| Natural variant | 131 | 1 | A → T in a breast cancer |
| | | | sample; somatic |
| | | | mutation. |
| Natural variant | 153 | 1 | N → S in HH16; |
| | | | phenotype consistent |
| | | | with Kallmann |
| | | | syndrome. |
| | | | Corresponds to variant |
| | | | rs139295139 |
| | | | [dbSNP \| Ensembl]. |
| Natural variant | 396 | 1 | P → S Found in a renal |
| | | | cell carcinoma sample; |
| | | | somatic mutation. |
| Natural variant | 400 | 1 | I → V in HH16; |
| | | | phenotype consistent |
| | | | with Kallmann |
| | | | syndrome; digenic; |
| | | | found in a patient also |
| | | | carrying mutation Cys- |
| | | | 268 in PROKR2. |
| | | | Corresponds to variant |
| | | | rs36026860 |
| | | | [dbSNP \| Ensembl]. |
| Natural variant | 435 | 1 | V → I in HH16; |
| | | | phenotype consistent |
| | | | with Kallmann |
| | | | syndrome; digenic; |
| | | | found in patients also |
| | | | carrying mutation Cys- |
| | | | 268 in PROKR2 or |
| | | | mutation Arg-687 in |
| | | | FGFR1. |
| | | | Corresponds to variant |
| | | | rs147436181 |
| | | | [dbSNP \| Ensembl]. |
| Natural variant | 688 | 1 | T → A in HH16; |
| | | | phenotype consistent |
| | | | with Kallmann |
| | | | syndrome; digenic; |
| | | | found in a patient also |
| | | | carrying mutation Asp- |
| | | | 217 in KAL1. |
| | | | Corresponds to variant |
| | | | rs318240751 |
| | | | [dbSNP \| Ensembl]. |
| Natural variant | 730 | 1 | R → Q in HH16; |
| | | | phenotype consistent |
| | | | with Kallmann |
| | | | syndrome. |
| | | | Corresponds to variant |
| | | | rs318240752 |
| | | | [dbSNP \| Ensembl]. |
| Natural variant | 733 | 1 | R → H in HH16; |
| | | | phenotype consistent |
| | | | with Kallmann |
| | | | syndrome. |
| | | | Corresponds to variant |
| | | | rs318240753 |
| | | | [dbSNP \| Ensembl]. |

Variants and fragments of neuropilins, plexins, and semaphorins are also disclosed and typically have at least 20 percent, 30 percent, 40 percent, 50 percent, 60 percent, 70 percent, 80 percent, 90 percent, 95 percent, 98 percent, 99 percent, sequence identity to a reference neuropilin, semaphorin or plexin, such as SEQ ID NOS:1, 2, 37-43, or 62-63.

### III. Antagonists of B7-H4 and B7-H4 Receptors

Antagonists of B7-H4 and B7-H4 receptors are disclosed. The disclosed antagonists are typically molecules that bind to or interact with B7-H4, a neuropilin, a plexin, a sempaphorin, or a combination thereof to reduce, block, or otherwise reduce or attenuate the binding between B7-H4 and a neuropilin, a plexin, a sempaphorin, or a combination thereof to reduce signal transduction that occurs as a consequence of B7-H4 binding to a neuropilin, a plexin, a sempaphorin, or a combination thereof. B7-H4 receptor antagonists inhibit, reduce, or block the biological activity of B7-H4 receptors. Accordingly, the antagonist can bind directly to a B7-H4 receptor and reduce or inhibit its ability to bind to B7-H4. Alternatively, the antagonist can bind to B7-H4, or another ligand of B7-H4 receptor such as a semaphorin, and block its ability to bind to the receptor Inhibitory nucleic acids that reduce expression of B7-H4 receptors and their ligands, are also disclosed.

Members of the B7 family of proteins have been shown to interact with multiple binding partners. Therefore, antagonists that bind to a B7-H4 or semaphorin ligand will allow neuropilin or plexin to bind with another ligand, and antagonists that bind a neuropilin or a plexin, or a combination thereof, will allow B7-H4 to bind with other receptors. Therefore, it may be beneficial to target only a ligand or receptor when trying to antagonize the B7-H4 pathway, so that interactions with other ligands/receptors are not disrupted. For example, if a neuropilin, a plexin, a combination thereof bind other ligands it may be beneficial to use an antagonist that binds to the B7-H4 ligand to selectively disrupt the activity of B7-H4, while allowing the neuropilin, the plexin, or combination thereof to bind other ligands and continue to function. Combining or using one or more antagonists that bind to both B7-H4 and one or more of a neuropilin, a plexin, or a sempaphorin, (e.g. bi-specific antibodies) may disrupt all interactions.

Signaling through B7-H4 receptors can limit, terminate or attenuate DC, T cell, B cell or neutrophil responses, preventing leukocyte hyper-activation and avoiding tissue and organ damage during immune responses. By inhibiting, reducing, or blocking the biological activity of B7-H4 receptors, B7-H4 antagonists maintain, prolong, or enhance activation of DCs, T cells, B cells or neutrophils Inhibiting, reducing, or blocking B7-H4 receptor biological activity can also inhibit the suppression or attenuation of T cell activation or functional activity that would otherwise occur. Furthermore, B7-H4 receptor antagonists may reduce the proliferation or differentiation of Tregs or reduce the activity of Tregs, such as a reduction in IL-10 production.

Exemplary antagonist include soluble neuropilin and plexin polypeptides and fragments and variants thereof, neuropilin and plexin fusion proteins, soluble fragments and variants of B7-H4, inhibitory antibodies that block the function of B7-H4, a neuropilin, a plexin, or a sempaphorin, and inhibitory nucleic acids that reduce the expression of B7-H4, a neuropilin, a plexin, or a sempaphorin.

A. B7-H4 Receptor Polypeptides

B7-H4 receptor polypeptides may be of any mammalian species of origin. In a preferred embodiment, the B7-H4 receptor polypeptide is of murine, non-human primate, or human origin.

Fragments and variants of B7-H4 receptors are also disclosed. Fragments and variants of B7-H4 receptor polypeptide can have the same activity, substantially the same activity, or different activity as a reference B7-H4 receptor polypeptide, for example a non-mutated B7-H4 receptor polypeptide. In some embodiments, the reference polypeptide is the full-length transmembrane protein. Substantially the same activity means it retains the ability to bind to B7-H4 alone or in combination with a semaphorin. Fragments and variants of neuropilin and plexin polypeptides typically have at least 20 percent, 30 percent, 40 percent, 50 percent, 60 percent, 70 percent, 80 percent, 90 percent, 95 percent, 98 percent, 99 percent, 100 percent, or more than 100 percent of the ability to bind to B7-H4 alone or in combination with a semaphorin compared to full-length neuropilin and plexin.

Receptor polypeptides, including fragments and variants thereof, typically bind the ligand and block the ability of ligands to bind to transmembrane neuropilin and plexin receptors or receptor complexes thereof and induce or maintain B7-H4 receptor mediated signal transduction. Accordingly, neuropilin and plexin polypeptides can block the ability of transmembrane neuropilin and plexin or receptor complexes thereof to mediate a decrease in T cell responses, a decrease in proliferation of T cells, a decrease in production and/or secretion of cytokines by T cells, a decrease in differentiation and effector functions of T cells and/or decreases in survival of T cells relative to T cells not contacted with the variant a receptor fusion polypeptide. In the case of Tregs, variant receptor fusion polypeptides can block the ability of transmembrane receptor to enhance Treg suppressive activity or increase the production of IL-10.

1. Fragments of B7-H4 Receptor Polypeptides

B7-H4 receptor polypeptides can be full-length polypeptides, or can be fragments of full-length B7-H4 receptor polypeptides. As used herein, a fragment of a B7-H4 receptor polypeptide refers to any subset of the polypeptide that is a shorter polypeptide of the full-length protein, although this may be formed into a fusion protein with a different protein or portion of a protein. Preferred fragments are fragments that retain the ability to bind to B7-H4.

Fragments of B7-H4 receptor polypeptides include soluble fragments. Soluble B7-H4 receptor polypeptide fragments are fragments of B7-H4 receptor polypeptides that may be shed, secreted or otherwise extracted from the producing cells. Soluble fragments of B7-H4 receptor polypeptides can include some or all of the extracellular domain of the receptor polypeptide, and lack some or all of the intracellular and/or transmembrane domain. It will be appreciated that fusion proteins, including Ig fusion proteins, such as those discussed in more detail below, can be soluble proteins. In some embodiments, the soluble protein includes a fragment of a neuropilin or a plexin and is not a fusion protein. In one embodiment, B7-H4 receptor polypeptide fragments include the entire extracellular domain of the receptor polypeptide, for example a neuropilin or a plexin. Various isoforms of neuropilins and plexins and domains and variants thereof are disclosed above. Furthermore, it will be appreciated that extracellular domains of B7-H4 receptor polypeptides can be readily determined by those of skill in the art using standard methodologies such as hydropathy plotting.

For example, an extracellular domain of NRP-1 can include amino acids 22-856 of SEQ ID NO:1, or fragment, variant, or alternative isoform thereof. An extracellular domain of Plexin4A include amino acids 24-1894 of SEQ ID NO:2, or fragment, variant, or alternative isoform thereof.

In some embodiments, a neuropilin or a plexin polypeptide fragment includes the entire extracellular domain of the neuropilin or the plexin polypeptide. In other embodiments, the soluble fragment of the neuropilin or the plexin includes a fragment of the extracellular domain that retains a biological activity of the neuropilin or the plexin. In preferred embodiments, the neuropilin or the plexin polypeptide or fragment thereof binds to B7-H4 and inhibits, block, prevents or otherwise reduces the ability of B7-H4 to bind to transmembrane neuropilin or plexin. It will be appreciated that the extracellular domain can include 1, 2, 3, 4, or 5 amino acids from the transmembrane domain. Alternatively, the extracellular domain can have 1, 2, 3, 4, or 5 amino acids removed from the C-terminus, N-terminus, or both.

B7-H4 receptor polypeptides or fragments thereof can be expressed from nucleic acids that include sequences that encode a signal sequence, also referred to as a signal peptide. The signal sequence is generally cleaved from the immature polypeptide to produce the mature polypeptide lacking the signal sequence. If the B7-H4 receptor polypeptide does not include a signal sequence, a heterologous signal sequence can be added. In other embodiments, the endogenous signal sequence of the receptor can be replaced by the signal sequence of another polypeptide using standard molecule biology techniques to affect the expression levels, secretion, solubility, or other property of the polypeptide.

In other embodiments, the soluble fragments of B7-H4 receptor polypeptides include fragments of the extracellular domain of a neuropilin or a plexin. In some embodiments, B7-H4 receptor polypeptide fragments include the portion of the extracellular domain that is necessary for binding to B7-H4.

In some embodiments, the soluble protein is a fusion protein, such as an Ig fusion protein. Any single or any combination of two or more extracellular domains or regions of a neuropilin or a plexin can be used to produce a fusion protein as discussed in more detail below.

2. Variants of B7-H4 Receptor Polypeptides

B7-H4 receptor polypeptides include polypeptides that are mutated to contain a deletion, substitution, insertion, or rearrangement of one or more amino acids relative to the wild-type polypeptide sequence. Variants can be variants of full-length neuropilin or a plexin, or fragments thereof such as those described above. In a preferred embodiment, the variant is a soluble fragment of a neuropilin or a plexin.

Variant B7-H4 receptor polypeptides can have any combination of amino acid substitutions, deletions or insertions. In one embodiment, isolated B7-H4 receptor variant polypeptides have an integer number of amino acid alterations such that their amino acid sequence shares at least 60, 70, 80, 85, 90, 95, 97, 98, 99, 99.5 or 99.9% identity with an amino acid sequence of a wild type B7-H4 receptor polypeptide. In a preferred embodiment, B7-H4 receptor polypeptides have an amino acid sequence sharing at least 60, 70, 80, 85, 90, 95, 97, 98, 99, 99.5 or 100% identity with the amino acid sequence of a wild type murine or wild type human B7-H4 receptor polypeptide, such as the sequences for the neuropilins or plexins disclosed above.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Natl. Acad. Sci. U.S.A., 89:10915-10919 (1992)) 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

Amino acid substitutions in B7-H4 receptor polypeptides may be “conservative” or “non-conservative”. As used herein, “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

Exemplary alternative isoforms and variants of neuropilins and plexins are described in the tables above with reference to conanical sequences provided. Useful variants include those that increase biological activity, as indicated by any of the assays shown/discussed, or that increase half-life or stability.

3. Modified B7-H4 Receptor Polypeptides

B7-H4 receptor polypeptides and fragments and variants thereof can be modified by chemical moieties that may be present in polypeptides in a normal cellular environment, for example, phosphorylation, methylation, amidation, sulfation, acylation, glycosylation, sumoylation and ubiquitylation. B7-H4 receptor polypeptides may also be modified with a label capable of providing a detectable signal, either directly or indirectly, including, but not limited to, radioisotopes and fluorescent compounds. The invention further concerns the embodiment of such molecules wherein the molecule is detectably labeled or comprises a conjugated toxin, drug, or enzyme for targeted therapy. This is particularly useful for targeting tumors that express high levels of B7-H4, such as ovarian and breast cancer.

B7-H4 receptor polypeptides and fragments and variants thereof may also be modified by chemical moieties that are not normally added to polypeptides in a cellular environment. Such modifications may be introduced into the molecule by reacting targeted amino acid residues of the polypeptide with an organic derivatizing agent that is capable of reacting with selected side chains or terminal residues. Another modification is cyclization of the protein.

Examples of chemical derivatives of the polypeptides include lysinyl and amino terminal residues derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate. Carboxyl side groups, aspartyl or glutamyl, may be selectively modified by reaction with carbodiimides (R—N═C═N—R′) such as 1-cyclohexyl-3-(2-morpholinyl-(4-ethyl)carbodiimide or 1-ethyl-3-(4-azonia-4,4-dimethylpentyl)carbodiimide. Furthermore, aspartyl and glutamyl residues can be converted to asparaginyl and glutaminyl residues by reaction with ammonia. Polypeptides may also include one or more D-amino acids that are substituted for one or more L-amino acids.

B. B7-H4 Receptor Fusion Proteins

B7-H4 receptor fusion proteins are also disclosed. In preferred embodiments, neuropilin, or plexin fusion proteins have the ability to bind to B7-H4 and function as B7-H4 receptor antagonists. Therefore, receptor fusion polypeptides typically block the ability of ligands to bind to transmembrane neuropilin or plexin receptors, or receptor complexes thereof, and induce or maintain B7-H4 receptor mediated signal transduction. Accordingly, neuropilin and plexin fusion proteins can block the ability of transmembrane neuropilin or plexin or a complex thereof to mediate a decrease in T cell responses, a decrease in proliferation of T cells, a decrease in production and/or secretion of cytokines by T cells, a decrease in differentiation and effector functions of T cells and/or decreases in survival of T cells relative to T cells not contacted with the variant a receptor fusion polypeptide. In the case of Tregs, variant receptor fusion polypeptides can block the ability of transmembrane receptor to enhance Treg suppressive activity or increase the production of IL-10.

B7-H4 receptor fusion polypeptides disclosed herein have a first fusion partner including all or a part of a B7-H4 receptor polypeptide fused (i) directly to a second polypeptide or, (ii) optionally, fused to a linker peptide sequence that is fused to the second polypeptide. Such fusion proteins may form dimers or multimers. The peptide/polypeptide linker domain can either be a separate domain, or alternatively can be contained within one of the other domains (B7-H4 receptor polypeptide or second polypeptide) of the fusion protein. Similarly, the domain that functions to dimerize or multimerize the fusion protein can either be a separate domain, or alternatively can be contained within one of the other domains (B7-H4 receptor polypeptide, second polypeptide or peptide/polypeptide linker domain) of the fusion protein. In one embodiment, the dimerization/multimerization domain and the peptide/polypeptide linker domain are the same.

Fusion proteins disclosed herein are of formula I:

N—R₁—R₂—R₃—C

wherein “N” represents the N-terminus of the fusion protein, “C” represents the C-terminus of the fusion protein. In the preferred embodiment, “R₁” is a B7-H4 receptor polypeptide, “R₂” is an optional peptide/polypeptide linker domain, and “R₃” is a second polypeptide. Alternatively, R₃ may be a B7-H4 receptor polypeptide and R₁ may be a second polypeptide.

Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric.

In one embodiment, the first fusion partner is a fragment of a neuropilin or a plexin. In a preferred embodiment, the fusion protein includes the extracellular domain of a neuropilin or a plexin, or a fragment thereof, and which is without the transmembrane domain, fused to an Ig Fc region. Recombinant B7-H4 receptor-Ig fusion proteins can be prepared by fusing the coding region of the extracellular domain of a neuropilin or a plexin or a fragment thereof to the Fc region of human IgG1 or mouse IgG2a, or other suitable Ig domain, as described previously (Chapoval, et al., Methods Mol. Med., 45:247-255 (2000)).

1. First Fusion Partner

The receptor fusion proteins can include full-length a neuropilin or a plexin, or can contain a fragment or variant of a full length a neuropilin or a plexin such as those discussed in more detail above. In preferred embodiment the first fusion partner is a soluble fragment of a neuropilin or a plexin, for example, part or all of the extracellular domain of a neuropilin or a plexin, or a variant thereof. Any mammalian sequence for a neuropilin or a plexin can be used. As an example, human sequences, as well as known isoforms and variants thereof, are provided in the sequences and tables above. In some embodiments, other mammalian sequences, such as mouse sequences, are known in the art and can be used. Human neuropilin or plexin polypeptides useful in the disclosed fusion proteins can be encoded by a nucleotide sequence having at least 80%, 85%, 90%, 95%, 99%, or 100% sequence identity to the nucleotide sequences referenced in the Accession Numbers listed above.

2. Dimerization and Multimerization Domains

The fusion proteins disclosed herein optionally contain a dimerization or multimerization domain that functions to dimerize or multimerize two or more fusion proteins. The domain that functions to dimerize or multimerize the fusion proteins can either be a separate domain, or alternatively can be contained within one of the other domains (B7-H4 polypeptide, B7-H4 receptor polypeptide, second polypeptide, or peptide/polypeptide linker domain) of the fusion protein.

a. Dimerization Domains

A “dimerization domain” is formed by the association of at least two amino acid residues or of at least two peptides or polypeptides (which may have the same, or different, amino acid sequences). The peptides or polypeptides may interact with each other through covalent and/or non-covalent association(s). Preferred dimerization domains contain at least one cysteine that is capable of forming an intermolecular disulfide bond with a cysteine on the partner fusion protein. The dimerization domain can contain one or more cysteine residues such that disulfide bond(s) can form between the partner fusion proteins. In one embodiment, dimerization domains contain one, two or three to about ten cysteine residues. In a preferred embodiment, the dimerization domain is the hinge region of an immunoglobulin. In this particular embodiment, the dimerization domain is contained within the linker peptide/polypeptide of the fusion protein.

Additional exemplary dimerization domain can be any known in the art and include, but not limited to, coiled coils, acid patches, zinc fingers, calcium hands, a C_{H}1-C_{L} pair, an “interface” with an engineered “knob” and/or “protruberance” as described in U.S. Pat. No. 5,821,333, leucine zippers (e.g., from jun and/or fos) (U.S. Pat. No. 5,932,448), SH2 (src homology 2), SH3 (src Homology 3) (Vidal, et al., Biochemistry, 43, 7336-44 ((2004)), phosphotyrosine binding (PTB) (Zhou, et al., Nature, 378:584-592 (1995)), WW (Sudol, Prog. Biochys. Mol. Bio., 65:113-132 (1996)), PDZ (Kim, et al., Nature, 378: 85-88 (1995); Komau, et al., Science, 269:1737-1740 (1995)) 14-3-3, WD40 (Hu, et al., J. Biol. Chem., 273, 33489-33494 (1998)) EH, Lim, an isoleucine zipper, a receptor dimer pair (e.g., interleukin-8 receptor (IL-8R); and integrin heterodimers such as LFA-1 and GPIIIb/IIIa), or the dimerization region(s) thereof, dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa, et al., J. Biol. Chem., 269(45): 27833-27839 (1994) and Radziejewski, et al., Biochem., 32(48): 1350 (1993)) and can also be variants of these domains in which the affinity is altered.

The polypeptide pairs can be identified by methods known in the art, including yeast two hybrid screens. Yeast two hybrid screens are described in U.S. Pat. Nos. 5,283,173 and 6,562,576. Affinities between a pair of interacting domains can be determined using methods known in the art, including as described in Katahira, et al., J. Biol. Chem., 277, 9242-9246 (2002)). Alternatively, a library of peptide sequences can be screened for heterodimerization, for example, using the methods described in WO 01/00814. Useful methods for protein-protein interactions are also described in U.S. Pat. No. 6,790,624.

b. Multimerization Domains

A “multimerization domain” is a domain that causes three or more peptides or polypeptides to interact with each other through covalent and/or non-covalent association(s). Suitable multimerization domains include, but are not limited to, coiled-coil domains. A coiled-coil is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled-coils with sequences including some irregular distribution of the 3 and 4 residues spacing are also contemplated. Hydrophobic residues are in particular the hydrophobic amino acids Val, Ile, Leu, Met, Tyr, Phe and Trp. Mainly hydrophobic means that at least 50% of the residues must be selected from the mentioned hydrophobic amino acids.

The coiled coil domain may be derived from laminin. In the extracellular space, the heterotrimeric coiled-coil protein laminin plays an important role in the formation of basement membranes. Apparently, the multifunctional oligomeric structure is required for laminin function. Coiled-coil domains may also be derived from the thrombospondins in which three (TSP-1 and TSP-2) or five (TSP-3, TSP-4 and TSP-5) chains are connected, or from COMP (COMPcc) (Guo, et at., EMBO J., 1998, 17: 5265-5272) which folds into a parallel five-stranded coiled coil (Malashkevich, et al., Science, 274: 761-765 (1996)).

Additional coiled-coil domains derived from other proteins, and other domains that mediate polypeptide multimerization are known in the art and are suitable for use in the disclosed fusion proteins.

C. B7-H4 Polypeptides

In some embodiments, the B7-H4 receptor antagonist is a B7-H4 polypeptide or fragment thereof. For example, soluble B7-H4 (sH4), also referred to herein as cell-free B7-H4, and circulating forms of B7-H4, has been detected in ovarian cancer patients as a potential biomarker, and results from a study of 68 patients with RA and 24 healthy volunteers indicated that soluble B7-H4 was present in blood of 65% of patients with RA, compared with only 13% of healthy people (Simon, et al., Cancer Res., 66(3):1570-5 (2006), Azuma, et al., PLoS Med., 6(10):e1000166 (2009). Epub 2009 Oct. 20). The levels of soluble B7-H4 were significantly higher in RA patients (96.1 ng/ml) compared to healthy people (<5 ng/ml).

In vivo studies in a mouse model indicate that both overexpression of sH4 and deletion of B7-H4 caused inflammation (Azuma, et al., PLoS Med., 6(10):e1000166 (2009). Epub 2009 Oct. 20). Symptoms in the mice appeared earlier and were more severe than controls, and inflammatory effects of soluble B7-H4 were shown to be dependent on neutrophils. B7-H4 fusion proteins that mimic the normal signaling by B7-H4, prevented disease development in the mice.

It is believed that soluble B7-H4 receptor polypeptides compete with endogenous B7-H4 receptors expressed on T cells for binding to natural ligands, including B7-H4, and therefore function to block the binding of B7-H4 to its receptor and/or antagonize B7-H4 receptor activation. Compositions and methods for using soluble B7-H4 to increase immune responses are disclosed in U.S. Published Application No. 2008/0206235 which is specifically incorporated by reference herein in its entirety. Soluble B7-H4 can be used to block, inhibit, or otherwise reduce signal transduction through B7-H4 receptors such as a neuropilin or a plexin.

The B7-H4 polypeptide can be of murine, non-human primate (Pan troglodytes, Macaca mulatta or Macaca fascicularis), or human origin. Murine B7-H4 polypeptides can have at least 80, 85, 90, 95 or 100% sequence identity to the B7-H4 polypeptide encoded by the nucleic acid having GenBank Accession Number NM_178594 or AY280973. Useful murine B7-H4 polypeptides have at least about 80, 85, 90, 95 or 100% sequence identity to the B7-H4 polypeptide according to GenBank Accession Number AAH32925.1 or NP_848709.2. Useful human B7-H4 polypeptides have at least about 80, 85, 90, 95 or 100% sequence identity to the B7-H4 polypeptide encoded by the nucleic acid having GenBank Accession Number AK026071. Useful human B7-H4 polypeptides have at least about 80, 85, 90, 95 or 100% sequence identity to the B7-H4 polypeptide according to GenBank Accession Number NP_078902.2 or BAB15349.1.

For example, human B7-H4 polypeptides can be encoded by a nucleotide sequence having at least 80%, 85%, 90%, 95%, 99%, or 100% sequence identity to:

| | |
|---|---|
| ATGGCTTCCC TGGGGCAGAT CCTCTTCTGG AGCATAATTA GCATCATCAT TATTCTGGCT | 60 |
| GGAGCAATTG CACTCATCAT TGGCTTTGGT ATTTCAGGGA GACACTCCAT CACAGTCACT | 120 |
| ACTGTCGCCT CAGCTGGGAA CATTGGGGAG GATGGAATCC TGAGCTGCAC TTTTGAACCT | 180 |
| GACATCAAAC TTTCTGATAT CGTGATACAA TGGCTGAAGG AAGGTGTTTT AGGCTTGGTC | 240 |
| CATGAGTTCA AAGAAGGCAA AGATGAGCTG TCGGAGCAGG ATGAAATGTT CAGAGGCCGG | 300 |
| ACAGCAGTGT TTGCTGATCA AGTGATAGTT GGCAATGCCT CTTTGCGGCT GAAAAACGTG | 360 |
| CAACTCACAG ATGCTGGCAC CTACAAATGT TATATCATCA CTTCTAAAGG CAAGGGGAAT | 420 |
| GCTAACCTTG AGTATAAAAC TGGAGCCTTC AGCATGCCGG AAGTGAATGT GGACTATAAT | 480 |
| GCCAGCTCAG AGACCTTGCG GTGTGAGGCT CCCCGATGGT TCCCCCAGCC CACAGTGGTC | 540 |
| TGGGCATCCC AAGTTGACCA GGGAGCCAAC TTCTCGGAAG TCTCCAATAC CAGCTTTGAG | 600 |
| CTGAACTCTG AGAATGTGAC CATGAAGGTT GTGTCTGTGC TCTACAATGT TACGATCAAC | 660 |
| AACACATACT CCTGTATGAT TGAAAATGAC ATTGCCAAAG CAACAGGGGA TATCAAAGTG | 720 |
| ACAGAATCGG AGATCAAAAG GCGGAGTCAC CTACAGCTGC TAAACTCAAA GGCTTCTCTG | 780 |
| TGTGTCTCTT CTTTCTTTGC CATCAGCTGG GCACTTCTGC CTCTCAGCCC TTACCTGATG | 840 |
| CTAAAATAA | 849 |

### (SEQ ID NO:3).

In some embodiments, a human B7-H4 polypeptide has at least 80%, 85%, 90%, 95%, 99%, or 100% sequence identity to:

MASLGQILFW SIISIIIILA GAIALIIGFG ISGRHSITVT
TVASAGNIGE DGILSCTFEP DIKLSDIVIQ WLKEGVLGLV
HEFKEGKDEL SEQDEMFRGR TAVFADQVIV GNASLRLKNV
QLTDAGTYKC YIITSKGKGN ANLEYKTGAF SMPEVNVDYN
ASSETLRCEA PRWFPQPTVV WASQVDQGAN FSEVSNTSFE
LNSENVTMKV VSVLYNVTIN NTYSCMIEND IAKATGDIKV
TESEIKRRSH LQLLNSKASL CVSSFFAISW ALLPLSPYLM
LK

### (SEQ ID NO:4).

The amino acid sequence of the human B7-H4 polypeptide of SEQ ID NO:4 without the signal sequence can be

GFGISGRHSI TVTTVASAGN IGEDGILSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSHLQLLNSK ASLCVSSFFA
ISWALLPLSP YLMLK

### (SEQ ID NO:5),

In some embodiments, a human B7-H4 polypeptide has at least 80%, 85%, 90%, 95%, 99%, or 100% sequence identity to:

MASLGQILFW SIISIIIILA GAIALIIGFG ISGRHSIIVT
TVASAGNIGE DGIQSCTFEP DIKLSDIVIQ WLKEGVLGLV
HEFKEGKDEL SEQDEMFRGR TAVFADQVIV GNASLRLKNV
QLTDAGTYKC YIIISKGKGN ANLEYKTGAF SMPEVNVDYN
ASSETLRCEA PRWFPQPTVV WASQVDQGAN FSEVSNISFE
LNSENVTMKV VSVLYNVTIN NTYSCMIEND IAKATGDIKV
TESEIKRRSH LQLLNSKASL CVSSFFAISW ALLPLSPYLM LK

### (SEQ ID NO:6).

The amino acid sequence of the human B7-H4 polypeptide of SEQ ID NO:6 without the signal sequence can be

GFGISGRHSI TVTTVASAGN IGEDGIQSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSHLQLLNSK ASLCVSSFFA
ISWALLPLSP YLMLK

### (SEQ ID NO:7).

Exemplary fragments and variants, including soluble fragments, that can be used as an antagonist of a neuropilin or a plexin mediated signal transduction pathway are discussed in more detail below.

1. Fragments of B7-H4 Polypeptides

The B7-H4 proteins contain two immunoglobulin domains within the extracellular domain, the IgV domain (or V domain) and the IgC domain (or C domain), which are related to the variable and constant domains of antibodies. The domains can be identified by anyone skilled in the art by searching against family and domain databases. Each Ig domain of the extracellular domain includes one disulfide bond formed between intradomain cysteine residues, as is typical for this fold and may be important for structure-function. In SEQ ID NOS: 4 and 6 these cysteines are located at residues 56 and 130 for the IgV domain, and 168 and 225 for the IgC domain.

For example, in some embodiments, the IgV domain includes a polypeptide having an amino acid sequence with 80%, 85%, 90%, 95%, 99%, or 100% sequence identity to the human amino acid sequence:

GFGISGRHSI TVTTVASAGN IGEDGIQSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVN

### (SEQ ID NO:8), or

HSITVTTVAS AGNIGEDGIQ SCTFEPDIKL SDIVIQWLKE
GVLGLVHEFK EGKDELSEQD EMFRGRTAVF ADQVIVGNAS
LRLKNVQLTD AGTYKCYIIT SKGKGNANLE YK

### (SEQ ID NO:9), or

GFGISGRHSI TVTTVASAGN IGEDGILSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVN

### (SEQ ID NO:10), or

HSITVTTVAS AGNIGEDGIL SCTFEPDIKL SDIVIQWLKE
GVLGLVHEFK EGKDELSEQD EMFRGRTAVF ADQVIVGNAS
LRLKNVQLTD AGTYKCYIIT SKGKGNANLE YK

### (SEQ ID NO:11),

In some embodiments, the IgC domain includes a polypeptide having an amino acid sequence with 80%, 85%, 90%, 95%, 99%, or 100% sequence identity to the human amino acid sequence:

VDYNASSETL RCEAPRWFPQ PTVVWASQVD QGANFSEVSN
TSFELNSENV TMKVVSVLYN VTINNTYSCM IENDIAKATG DIKVT

### (SEQ ID NO:12), or

PEVNVDYNAS SETLRCEAPR WFPQPTVVWA SQVDQGANFS
EVSNTSFELN SENVTMKVVS VLYNVTINNT YSCMIENDIA
KATGDIKVT

### (SEQ ID NO:13),

FSMPEVNVDY NASSETLRCE APRWFPQPTV VWASQVDQGA
NFSEVSNTSF ELNSENVTMK VVSVLYNVTI NNTYSCMIEN
DIAKATGDIK VTESEIKRRS

### (SEQ ID NO:14),

VNVDYNASSE TLRCEAPRWF PQPTVVWASQ VDQGANFSEV
SNTSFELNSE NVTMKVVSVL YNVTINNTYS CMIENDIAKA
TGDIKVTESE IKRRS

### (SEQ ID NO:15),

YNASSETLRC EAPRWFPQPT VVWASQVDQG ANFSEVSNTS
FELNSENVTM KVVSVLYNVT INNTYSCMIE NDIAKATGDI
KVTESEIKRR S

### (SEQ ID NO:16),

In some embodiments, a fragment of B7-H4 includes the IgV and IgC domains, but does not include the entire extracellular domain. For example, in some embodiments, a fragment of full-length B7-H4 includes a polypeptide having an amino acid sequence with 80%, 85%, 90%, 95%, 99%, or 100% sequence identity to the human amino acid sequence:

GFGISGRHSI TVTTVASAGN IGEDGIQSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKAIGD IKVTESEIKR RS

### (SEQ ID NO:17), or

GFGISGRHSI TVTTVASAGN IGEDGILSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RS

### (SEQ ID NO:18),

In addition, there is one predicted N-linked glycosylation site in the IgV domain and six predicted glycosylation sites in the IgC domain, which are conserved between mouse and human B7-H4 sequences.

Fragments of B7-H4 polypeptides include cell free fragments. Cell free B7-H4 polypeptide fragments are fragments of B7-H4 polypeptides that may be shed, secreted or otherwise extracted from the producing cells. Cell free fragments of B7-H4 polypeptides can include some or all of the extracellular domain of the polypeptide, and lack some or all of the intracellular and/or transmembrane domains. In one embodiment, B7-H4 polypeptide fragments include the entire extracellular domain of the B7-H4 polypeptide. In other embodiments, the cell free fragments of B7-H4 polypeptides include fragments of the extracellular domain that retain B7-H4 biological activity. The extracellular domain can include 1, 2, 3, 4, or 5 contiguous amino acids from the transmembrane domain, and/or 1, 2, 3, 4, or 5 contiguous amino acids from the signal sequence. Alternatively, the extracellular domain can have 1, 2, 3, 4, 5 or more amino acids removed from the C-terminus, N-terminus, or both

Generally, the B7-H4 polypeptides or fragments thereof are expressed from nucleic acids that include sequences that encode a signal sequence. The signal sequence is generally cleaved from the immature polypeptide to produce the mature polypeptide lacking the signal sequence. SEQ ID NOs: 5 and 7 each lack a signal peptide. The signal sequence of B7-H4, and optionally, one, two, three, four, five, or more amino acids of the IgV domain can be replaced by the signal sequence of another polypeptide using standard molecule biology techniques to affect the expression levels, secretion, solubility, or other property of the polypeptide. The signal sequence that is used to replace the B7-H4 signal sequence can be any known in the art. SEQ ID NOs: 4 and 6 each contain the endogenous B7-H4 signal peptide, which is from amino acid 1 to about amino acid 24 of SEQ ID NO:4 and 6, see for example UniProtKB/Swiss-Prot: Q7Z7D3.1.

B7-H4 polypeptides and receptor polypeptides, and fragments and fusions thereof, both with and without a signal sequence are provided herein. It is understood that the mature protein, i.e., the protein sequence without the signal sequence, is a putative mature protein. During normal cell expression, a signal sequence can be removed by a cellular peptidase to yield a mature protein. The actual mature protein expressed following in vivo cleavage of the signal sequence many include 1, 2, 3, 4, 5, 6, 7, or 8 more; or 1, 2, 3, 4, 5, 6, 7, or 8 fewer amino acids than the putative mature proteins provided herein. It is also understood that a nucleic acid sequence encoding the putative mature proteins provided herein can be modified to include a nucleic acid sequence encoding an endogenous or heterologous signal sequence at the 5′ end, which, when expressed in a cell, yields a mature B7-H4 protein, or fragment, or fusion thereof such as those putative mature proteins provided herein.

2. Variants of B7-H4 Polypeptides

B7-H4 polypeptides include polypeptides that are mutated to contain a deletion, substitution, insertion, or rearrangement of one or more amino acids relative to the wild-type polypeptide sequence. Variants can be variants of full-length B7-H4, or fragments thereof such as those described above. In a preferred embodiment, the variant is a soluble fragment of B7-H4.

Useful variants include those that increase biological activity, as indicated by any of the assays described herein, or that increase half-life or stability of the protein. In a preferred embodiment, the B7-H4 polypeptide or fragment has been modified with at least one amino acid substitution, deletion, or insertion that increases the binding of the molecule to a B7-H4 receptor such as a neuropilin or a plexin, or decrease bind to a semaphorin.

Other preferred variants are those B7-H4 polypeptides that are engineered to selectively bind to one type of T cell versus other immune cells. Preferential binding refers to binding that is at least 10%, 20%, 30%, 40%, 50%, 60%, 70%, 80%, 90%, 95%, or greater for one type of cell over another type of cell.

Still other variants of B7-H4 can be engineered to have reduced binding to immune cells relative to wildtype B7-H4, or have reduced binding to a neuropilin or a plexin while leaving other interactions of B7-H4 intact, such as a variant that modifies the IgC domain. These variants can be used in combination with variants having stronger binding properties to modulate the immune response with a moderate impact.

Finally, variant B7-H4 polypeptides can be engineered to have an increased half-life relative to wildtype. These variants typically are modified to resist enzymatic degradation. Exemplary modifications include modified amino acid residues and modified peptide bonds that resist enzymatic degradation. Various modifications to achieve this are known in the art. For example, the juxtamembrane region of B7-H4 includes a dibasic motif, KRRS, which could potentially be recognized and cleaved, for example by a member of the proprotein convertase family of proteases. This motif (KRRS) can be removed, blocked, or modified to increase half-life. The variants can be modified to adjust for effects of affinity for the receptor on the half-life of B7-H4 polypeptides, fragments, or fusions thereof at serum and endosomal pH.

Variant B7-H4 polypeptides can have any combination of amino acid substitutions, deletions or insertions. In one embodiment, isolated B7-H4 polypeptides have an integer number of amino acid alterations such that their amino acid sequence shares at least 60, 70, 80, 85, 90, 95, 97, 98, 99, 99.5 or 99.9% identity with an amino acid sequence of a wild type B7-H4 polypeptide, such as those provided above. In a preferred embodiment, B7-H4 polypeptides have an amino acid sequence sharing at least 60, 70, 80, 85, 90, 95, 97, 98, 99, 99.5 or 100% identity with the amino acid sequence of a wild type murine or wild type human B7-H4 receptor polypeptide, such as the sequences for B7-H4 provided above.

Amino acid substitutions in B7-H4 polypeptides can be “conservative” or “non-conservative”. Conservative and non-conservative substitutions as well as methods of determining percent identity are discussed in detail above with respect to B7-H4 receptor polypeptides.

D. Antibodies

In some embodiments, a B7-H4 receptor antagonist is an antibody, or an antigen-binding fragment thereof. Methods of producing antibodies are well known and within the ability of one of ordinary skill in the art and are described in more detail below.

B7-H4 receptor antagonistic antibodies disclosed herein specifically bind to and block a B7-H4 receptor and are capable of reducing or inhibiting the binding of B7-H4 receptors to B7-H4. In some embodiments, the B7-H4 receptor antagonistic antibody is an antibody or antigen-binding fragment thereof that binds to a ligand of the B7-H4 receptor and blocks the ability of the ligand to bind to, or otherwise activate receptor activity. These antibodies are defined as “blocking”, “function-blocking” or “antagonistic” antibodies. In preferred embodiments the antagonistic antibodies specifically bind to a portion of the extracellular domain of B7-H4 receptors or the extracellular domain of B7-H4.

For example, the antibody can be an antibody or an antigen-binding fragment thereof that binds to a neuropilin, a plexin, or a complex thereof.

The antibody can be an antibody that binds to a ligand of the receptor. For example, the antibody can be an anti-B7-H4 antibody, or an antibody that binds to a sempaphorin.

The antibody can be a bi-specific antibody. For example, a bi-specific antibody specific for two or more B7-H4 receptor epitopes; two or more B7-H4 epitopes; or a ligand epitope and a receptor epitope can be generated using methods generally known in the art. Homodimeric antibodies can also be generated by cross-linking techniques known in the art (e.g., Wolff et al., Cancer Res., 53: 2560-2565 (1993)).

In some embodiments, one or more antagonistic antibodies are administered in combination. For example, a method of antagonizing a neuropilin, a plexin, or complex thereof can include co-administration of two or more antibodies or an antigen binding fragments thereof that bind wherein the two antibodies separately bind to two or more of a neuropilin, a plexin, a semaphorin, and B7-H4. In a particular embodiment, an antibody or an antigen-binding fragment thereof that binds a neuropilin, a plexin, or a semaphorin is co-administered with an anti-B7-H4 antibody or an antigen-binding fragment thereof.

E. Inhibitory Nucleic Acids

In another embodiment B7-H4 receptor antagonists reduce B7-H4 binding to a B7-H4 receptor by reducing or inhibiting the expression of B7-H4 receptors or B7-H4. Useful B7-H4 receptor antagonists that reduce or inhibit expression of B7-H4 receptors include functional nucleic acids, including, but not limited to, antisense oligonucleotides, ribozymes, external guide sequences, triplex-forming oligonucleotides (TFOs), aptamers, RNAi, siRNA, and microRNA specific for receptor nucleic acids or proteins.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10-12 M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000-fold difference in binding affinities between the target molecule and another molecule that differ at only a single position on the molecule. It is preferred that the aptamer have a K_{d} with the target molecule at least 10-, 100-, 1000-, 10,000-, or 100,000-fold lower than the K_{d} with a background binding molecule. It is preferred when doing the comparison for a polypeptide for example, that the background molecule be a different polypeptide.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed transferases.

Inhibitory nucleic acids and methods of producing them are well known in the art. siRNA design software is available for example at http://i.cs.hku.hk/˜sirna/software/sirna.php. Synthesis of nucleic acids is well known in the art. See, for example, Molecular Cloning: A Laboratory Manual (Sambrook and Russel eds. 3rd ed.) Cold Spring Harbor, N.Y. (2001). The term “siRNA” means a small interfering RNA that is a short-length double-stranded RNA that is not toxic. Generally, there is no particular limitation in the length of siRNA as long as it does not show toxicity. “siRNAs” can be, for example, 15 to 49 bp, preferably 15 to 35 bp, and more preferably 21 to 30 bp long. Alternatively, the double-stranded RNA portion of a final transcription product of siRNA to be expressed can be, for example, 15 to 49 bp, preferably 15 to 35 bp, and more preferably 21 to 30 bp long. The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. Nonpairing portions can be contained to the extent that they do not interfere with siRNA formation.

The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, the double-stranded RNA region of siRNAs in which two RNA strands pair up may contain both bulge and mismatched, which sum up to, preferably 1 to 7, more preferably 1 to 5 in number.

The terminal structure of siRNA may be either blunt or cohesive (overhanging) as long as siRNA can silence, reduce, or inhibit the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 bp double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the siRNA is not necessarily the cut off structure at both ends as described above, and may have a stem-loop structure in which ends of one side of double-stranded RNA are connected by a linker RNA. The length of the double-stranded RNA region (stem-loop portion) can be, for example, 15 to 49 bp, preferably 15 to 35 bp, and more preferably 21 to 30 bp long. Alternatively, the length of the double-stranded RNA region that is a final transcription product of siRNAs to be expressed is, for example, 15 to 49 bp, preferably 15 to 35 bp, and more preferably 21 to 30 bp long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA or viral RNA, or an artificial RNA molecule.

miRNAs are produced by the cleavage of short stem-loop precursors by Dicer-like enzymes; whereas, siRNAs are produced by the cleavage of long double-stranded RNA molecules. MiRNAs are single-stranded, whereas siRNAs are double-stranded.

Useful functional nucleic acids include those that reduce the expression of RNA encoding B7-H4 receptors or a receptor ligand such as a semaphorin by at least 20%, 30%, 40%, 50%, 60%, 70%, 80%, 90% or 95% compared to controls. For example, in some embodiments, the inhibitory nucleic acid reduces expression of RNA encoding a neuropilin, a plexin, a sempaphorin or a fragment or variant thereof of with 70%, 75%, 80%, 85%, 90%, 95%, or 99% sequence identity to the neuropilin, plexin, or semaphorin. For example the target can be a nucleic acid that encodes a protein sequence of a neuropilin, a plexin, or a semaphorin provided above. Expression of B7-H4 receptors and ligands thereof can be measured by methods well know to those of skill in the art, including northern blotting and quantitative polymerase chain reaction (PCR). In a preferred embodiment, the inhibitory nucleic acids lead to reduced expression of a neuropilin, plexin, or semaphorin, for example any of the neuropilin, plexin, or semaphorin amino acid sequences disclosed above.

### IV. Agonists of B7-H4 Receptor Signal Transduction

Agonists of B7-H4 receptors are disclosed. The disclosed agonists are typically molecules that bind to or interact with a neurtropilin, a plexin, or a complex thereof. Agonists can promote, induce, or otherwise increase or enhance an immune suppressive response, such as an immune suppressive response transduced through a neuropilin, a plexin, or a complex thereof.

B7-H4 receptor agonists function to stimulate the biological activity of B7-H4 receptors. B7-H4 receptor agonists can decrease T cell responses, decrease proliferation of T cells, decrease production and/or secretion of cytokines by T cells such as Th1 and Th17 cytokines, decrease differentiation and effector functions of T cells and/or decreases survival of T cells relative to T cells not contacted with the B7-H4 receptor agonist. Additionally or alternatively, a B7-H4 receptor agonist may increase Treg numbers or differentiation, or increase Treg cellular responses, such as an increase in IL-10 production, and restore immune tolerance. The agonist can mimic, promote or increase binding of B7-H4 to a semaphorin, a neuropilin, a plexin, or a complex thereof. For example, the agonist can bind to a neuropilin, a plexin, or a complex thereof and induces signal transduction through the B7-H4 receptor.

Exemplary agonists include B7-H4 fusion proteins, semphorins, and function activating antibodies that bind to B7-H4 receptors or ligands thereof such as B7-H4 and semphorin, or a combination thereof.

The agonists can contain a targeting domain to target the molecule to specific sites in the body. Preferred targeting domains target the agonist to areas of inflammation. Exemplary targeting domains are antibodies, or antigen binding fragments thereof that are specific for inflamed tissue or to a proinflammatory cytokine including but not limited to IL17, IL-4, IL-6, IL-12, IL-21, IL-22, and IL-23. Additional targeting domains can be peptide aptamers specific for a proinflammatory cytokine. In other embodiments, the agonist can include binding partner specific for a polypeptide displayed on the surface of an immune cell, for example a T cell. In still other embodiments, the targeting domain specifically targets activated immune cells. Preferred immune cells that are targeted include Th0, Th1, and Th17 T cells.

A. B7-H4 Fusion Proteins

B7-H4 polypeptides, fusions, and pharmaceutical compositions including B7-H4 polypeptides, and fragments and fusions thereof are disclosed in U.S. Published Application Nos. 2012/0177645 and 2012/0276095 which are incorporated herein by reference in their entirety.

B7-H4 fusion polypeptides have a first fusion partner including all or a part of a B7-H4 protein fused to a second polypeptide directly or via a linker peptide sequence that is fused to the second polypeptide. The fusion proteins optionally contain a domain that functions to dimerize or multimerize two or more fusion proteins. The peptide/polypeptide linker domain can either be a separate domain, or alternatively can be contained within one of the other domains (B7-H4 polypeptide or second polypeptide) of the fusion protein. Similarly, the domain that functions to dimerize or multimerize the fusion proteins can either be a separate domain, or alternatively can be contained within one of the other domains (B7-H4 polypeptide, second polypeptide or peptide/polypeptide linker domain) of the fusion protein. In one embodiment, the dimerization/multimerization domain and the peptide/polypeptide linker domain are the same.

Fusion proteins disclosed herein are of formula I:

N—R₁—R₂—R₃—C

wherein “N” represents the N-terminus of the fusion protein, “C” represents the C-terminus of the fusion protein. In the preferred embodiment, “R₁” is a B7-H4 polypeptide, “R₂” is an optional peptide/polypeptide linker domain, and “R₃” is a second polypeptide. Alternatively, R₃ may be a B7-H4 polypeptide and R₁ may be a second polypeptide.

Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric. Dimerization and multimerization domains are discussed in detail above with respect to B7-H4 receptor fusions proteins.

In one embodiment, the first fusion partner is a fragment or variant of B7-H4, such as those discussed above. In a preferred embodiment, the fusion protein includes the extracellular domain of B7-H4, or a fragment thereof, and which is without the transmembrane domain, fused to an Ig Fc region. Recombinant B7-H4-Ig fusion proteins can be prepared by fusing the coding region of the extracellular domain of B7-H4 or a fragment thereof to the Fc region of human IgG1 or mouse IgG2a, or other suitable Ig domain, as described previously (Chapoval, et al., Methods Mol. Med., 45:247-255 (2000)).

Exemplary B7-H4 fusion proteins are provided. In some embodiments, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGIQSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKTG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIKRR
SEPKSCDKTH TCPPCPAPEL LGGPSVFLFP PKPKDTLMIS
RTPEVTCVVV DVSHEDPEVK FNWYVDGVEV HNAKTKPREE
QYNSTYRVVS VLTVLHQDWL NGKEYKCKVS NKALPAPIEK
TISKAKGQPR EPQVYTLPPS RDELTKNQVS LTCLVKGFYP
SDIAVEWESN GQPENNYKTT PPVLDSDGSF FLYSKLTVDK
SRWQQGNVFS CSVMHEALHN HYTQKSLSLS PGK

### (SEQ ID NO:22).

The amino acid sequence of the human B7-H4 fusion protein of SEQ ID NO:22 without the signal sequence can be:

GFGISGRHSI TVTTVASAGN IGEDGIQSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SRDELTKNQV SLTCLVKGFY PSDIAVEWES NGQPENNYKT
TPPVLDSDGS FFLYSKLTVD KSRWQQGNVF SCSVMHEALH
NHYTQKSLSL SPGK

### (SEQ ID NO:23).

In another embodiment, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGIQSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKTG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIKRR
SDKTHTCPPC PAPELLGGPS VFLFPPKPKD TLMISRTPEV
TCVVVDVSHE DPEVKFNWYV DGVEVHNAKT KPREEQYNST
YRVVSVLTVL HQDWLNGKEY KCKVSNKALP APIEKTISKA
KGQPREPQVY TLPPSRDELT KNQVSLTCLV KGFYPSDIAV
EWESNGQPEN NYKTTPPVLD SDGSFFLYSK LTVDKSRWQQ
GNVFSCSVMH EALHNHYTQK SLSLSPGK

### (SEQ ID NO:44).

The amino acid sequence of the human B7-H4 fusion protein of SEQ ID NO:44 without the signal sequence can be:

GFGISGRHSI TVTTVASAGN IGEDGIQSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSDKTHTCPP CPAPELLGGP
SVFLFPPKPK DTLMISRTPE VTCVVVDVSH EDPEVKFNWY
VDGVEVHNAK IKPREEQYNS TYRVVSVLTV LHQDWLNGKE
YKCKVSNKAL PAPIEKTISK AKGQPREPQV YTLPPSRDEL
TKNQVSLTCL VKGFYPSDIA VEWESNGQPE NNYKTTPPVL
DSDGSFFLYS KLTVDKSRWQ QGNVFSCSVM HEALHNHYTQ
KSLSLSPGK

### (SEQ ID NO:45).

In another embodiment, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGIQSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKTG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIDKT
HTCPPCPAPE LLGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SRDELTKNQV SLTCLVKGFY PSDIAVEWES
NGQPENNYKT TPPVLDSDGS FFLYSKLTVD KSRWQQGNVF
SCSVMHEALH NHYTQKSLSL SPGK

### (SEQ ID NO:46).

The amino acid sequence of the human B7-H4 fusion protein of SEQ ID NO:46 without the signal sequence can be:

GFGISGRHSI TVTTVASAGN IGEDGIQSCT FEPDIKLSDI
VIQWLKEGVL GLVHEFKEGK DELSEQDEMF RGRIAVFADQ
VIVGNASLRL KNVQLIDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIDK THTCPPCPAP ELLGGPSVFL
FPPKPKDTLM ISRTPEVTCV VVDVSHEDPE VKFNWYVDGV
EVHNAKTKPR EEQYNSTYRV VSVLTVLHQD WLNGKEYKCK
VSNKALPAPI EKTISKAKGQ PREPQVYTLP PSRDELTKNQ
VSLTCLVKGF YPSDIAVEWE SNGQPENNYK TTPPVLDSDG
SFFLYSKLTV DKSRWQQGNV FSCSVMHEAL HNHYTQKSLS
LSPGK

### (SEQ ID NO:47).

In another embodiment, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGIQSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKTG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIKRR
SHLQLLNSKD KTHTCPPCPA PELLGGPSVF LFPPKPKDTL
MISRTPEVTC VVVDVSHEDP EVKFNWYVDG VEVHNAKTKP
REEQYNSTYR VVSVLTVLHQ DWLNGKEYKC KVSNKALPAP
IEKTISKAKG QPREPQVYTL PPSRDELTKN QVSLTCLVKG
FYPSDIAVEW ESNGQPENNY KTTPPVLDSD GSFFLYSKLT
VDKSRWQQGN VFSCSVMHEA LHNHYTQKSL SLSPGK

### (SEQ ID NO:48).

The amino acid sequence of the human B7-H4 fusion protein of SEQ ID NO:48 without the signal sequence can be:

GFGISGRHSI TVTTVASAGN IGEDGIQSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSHLQLLNSK DKTHTCPPCP
APELLGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSRDELTK NQVSLTCLVK GFYPSDIAVE WESNGQPENN
YKTTPPVLDS DGSFFLYSKL TVDKSRWQQG NVFSCSVMHE
ALHNHYTQKS LSLSPGK

### (SEQ ID NO:49).

In another embodiment, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGILSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKTG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIKRR
SEPKSCDKTH TCPPCPAPEL LGGPSVFLFP PKPKDTLMIS
RTPEVTCVVV DVSHEDPEVK FNWYVDGVEV HNAKTKPREE
QYNSTYRVVS VLTVLHQDWL NGKEYKCKVS NKALPAPIEK
TISKAKGQPR EPQVYTLPPS RDELTKNQVS LTCLVKGFYP
SDIAVEWESN GQPENNYKTT PPVLDSDGSF FLYSKLTVDK
SRWQQGNVFS CSVMHEALHN HYTQKSLSLS PGK

### (SEQ ID NO:24).

The amino acid sequence of the human B7-H4 fusion protein of SEQ ID NO:24 without the signal sequence can be:

GFGISGRHSI TVTTVASAGN IGEDGILSCT FEPDIKLSDT
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SRDELTKNQV SLTCLVKGFY PSDIAVEWES NGQPENNYKT
TPPVLDSDGS FFLYSKLTVD KSRWQQGNVF SCSVMHEALH
NHYTQKSLSL SPGK

### (SEQ ID NO:25).

Changes in SEQ ID NO:24 relative to SEQ ID NO:22 are illustrated below:

MEWSWVFLFFLSVTTGVHSGFGISGRHSITVTTVASAGNIGEDGI [symbol] SC
TFEPDIKLSDIVIQWLKEGVLGLVHEFKEGKDELSEQDEMFRGRTAVFAD
QVIVGNASLRLKNVQLTDAGTYKCYIITSKGKGNANLEYKTGAFSMPEVN
VDYNASSETLRCEAPRWFPQPTVVWASQVDQGANFSEVSNTSFELNSENV
TMKVVSVLYNVTINNTYSCMIENDIAKATGDIKVTESEIKRRSEPKSCDK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNV
FSCSVMHEALHNHYTQKSLSLSPGK

### (SEQ ID NO:64).

In another embodiment, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGILSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKTG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIKRR
SDKTHTCPPC PAPELLGGPS VFLFPPKPKD TLMISRTPEV
TCVVVDVSHE DPEVKFNWYV DGVEVHNAKT KPREEQYNST
YRVVSVLTVL HQDWLNGKEY KCKVSNKALP APIEKTISKA
KGQPREPQVY TLPPSRDELT KNQVSLTCLV KGFYPSDIAV
EWESNGQPEN NYKTTPPVLD SDGSFFLYSK LTVDKSRWQQ
GNVFSCSVMH EALHNHYTQK SLSLSPGK

### (SEQ ID NO:50).

The amino acid sequence of the human B7-H4 fusion protein of SEQ ID NO:50 without the signal sequence can be:

GFGISGRHSI TVTTVASAGN IGEDGILSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSDKTHTCPP CPAPELLGGP
SVFLFPPKPK DTLMISRTPE VTCVVVDVSH EDPEVKFNWY
VDGVEVHNAK TKPREEQYNS TYRVVSVLTV LHQDWLNGKE
YKCKVSNKAL PAPIEKTISK AKGQPREPQV YTLPPSRDEL
TKNQVSLTCL VKGFYPSDIA VEWESNGQPE NNYKTTPPVL
DSDGSFFLYS KLTVDKSRWQ QGNVFSCSVM HEALHNHYTQ
KSLSLSPGK

### (SEQ ID NO:51).

Changes in SEQ ID NO:50 relative to SEQ ID NO:22 are show below:

MEWSWVFLFFLSVTTGVHSGFGISGRHSITVTTVASAGNIGEDGI [symbol] SC
TFEPDIKLSDIVIQWLKEGVLGLVHEFKEGKDELSEQDEMFRGRTAVFAD
QVIVGNASLRLKNVQLTDAGTYKCYIITSKGKGNANLEYKYGAFSMPEVN
VDYNASSETLRCEAPRWFPQPTVVWASQVDQGANFSEVSNTSFELNSENV
TMKVVSVLYNVTINNTYSCMIENDIAKATGDIKVTESEIKRRS [symbol] DKTH
TCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFS
CSVMHEALHNHYTQKSLSLSPGK

(SEQ ID NO:66). Amino acids in strike-through are deleted.

In another embodiment, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGILSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKYG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIDKT
HICPPCPAPE LLGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SRDELTKNQV SLTCLVKGFY PSDIAVEWES
NGQPENNYKT IPPVLDSDGS FFLYSKLTVD KSRWQQGNVF
SCSVMHEALH NHYTQKSLSL SPGK

### (SEQ ID NO:52).

The amino acid sequence of the human B7-H4 fusion protein of SEQ ID NO:52 without the signal sequence can be:

GFGISGRHSI TVTTVASAGN IGEDGILSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIDK THTCPPCPAP ELLGGPSVFL
FPPKPKDTLM ISRTPEVTCV VVDVSHEDPE VKFNWYVDGV
EVHNAKTKPR EEQYNSTYRV VSVLTVLHQD WLNGKEYKCK
VSNKALPAPI EKTISKAKGQ PREPQVYTLP PSRDELTKNQ
VSLTCLVKGF YPSDIAVEWE SNGQPENNYK TTPPVLDSDG
SFFLYSKLTV DKSRWQQGNV FSCSVMHEAL HNHYTQKSLS
LSPGK

### (SEQ ID NO:53).

Changes in SEQ ID NO:52 relative to SEQ ID NO:22 are illustrated below:

MEWSWVFLFFLSVTTGVHSGFGISGRHSITVTTVASAGNIGEDGI [symbol] SC
TFEPDIKLSDIVIQWLKEGVLGLVHEFKEGKDELSEQDEMFRGRTAVFAD
QVIVGNASLRLKNVQLTDAGTYKCYIITSKGKGNANLEYKTGAFSMPEVN
VDYNASSETLRCEAPRWFPQPTVVWASQVDQGANFSEVSNTSFELNSENV
TMKVVSVLYNVTINNTYSCMIENDIAKATGDIKVTESEI [symbol] DKTHTCP
PCPAPELLGGPSVFLFPPKPKDTLMISRTPEVTCVVVDVSHEDPEVKFNW
YVDGVEVHNAKIKPREEQYNSIYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPGK

(SEQ ID NO:67). Amino acid sequences in strike-through are deleted.

In another embodiment, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGILSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKTG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIKRR
SHLQLLNSKD KTHTCPPCPA PELLGGPSVF LFPPKPKDTL
MISRTPEVTC VVVDVSHEDP EVKFNWYVDG VEVHNAKTKP
REEQYNSTYR VVSVLTVLHQ DWLNGKEYKC KVSNKALPAP
IEKTISKAKG QPREPQVYTL PPSRDELTKN QVSLTCLVKG
FYPSDIAVEW ESNGQPENNY KTTPPVLDSD GSFFLYSKLT
VDKSRWQQGN VFSCSVMHEA LHNHYTQKSL SLSPGK

### (SEQ ID NO:54).

The amino acid sequence of the human B7-H4 fusion protein of SEQ ID NO:54 without the signal sequence can be:

GFGISGRHSI IVTTVASAGN IGEDGILSCI FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSHLQLLNSK DKTHTCPPCP
APELLGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSRDELTK NQVSLTCLVK GFYPSDIAVE WESNGQPENN
YKTTPPVLDS DGSFFLYSKL TVDKSRWQQG NVFSCSVMHE
ALHNHYTQKS LSLSPGK

### (SEQ ID NO:55).

Changes in SEQ ID NO:54 relative to SEQ ID NO:22 are illustrated below:

MEWSWVFLFFLSVTTGVHSGFGISGRHSITVTTVASAGNIGEDGI [symbol] SCTF
EPDIKLSDIVIQWLKEGVLGLVHEFKEGKDELSEQDEMFRGRTAVFADQV
IVGNASLRLKNVQLTDAGTYKCYIITSKGKGNANLEYKTGAFSMPEVNVD
YNASSETLRCEAPRWFPQPTVVWASQVDQGANFSEVSNTSFELNSENVTM
KVVSVLYNVTINNTYSCMIENDIAKATGDIKVTESEIKRRS [symbol]
DKTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVM
HEALHNHYTQKSLSLSPGK

(SEQ ID NO:68). Amino acid sequences in strike-through are deleted.

In another embodiment, a representative human B7-H4 fusion protein has at least 80%, 85%, 90%, 95%, 99% or 100% sequence identity to:

MEWSWVFLFF LSVTTGVHSG FGISGRHSIT VTTVASAGNI
GEDGILSCTF EPDIKLSDIV IQWLKEGVLG LVHEFKEGKD
ELSEQDEMFR GRTAVFADQV IVGNASLRLK NVQLTDAGTY
KCYIITSKGK GNANLEYKTG AFSMPEVNVD YNASSETLRC
EAPRWFPQPT VVWASQVDQG ANFSEVSNTS FELNSENVTM
KVVSVLYNVT INNTYSCMIE NDIAKATGDI KVTESEIKRR
SEPKSCDKTH TCPPCPAPEL LGGPSVFLFP PKPKDTLMIS
RTPEVTCVVV DVSHEDPEVK FNWYVDGVEV HNAKTKPREE
QYQSTYRVVS VLTVLHQDWL NGKEYKCKVS NKALPAPIEK
TISKAKGQPR EPQVYTLPPS RDELTKNQVS LTCLVKGFYP
SDIAVEWESN GQPENNYKTT PPVLDSDGSF FLYSKLTVDK
SRWQQGNVFS CSVMHEALHN HYTQKSLSLS PGK

### (SEQ ID NO:56).

The amino acid sequence of the human B7-H4 fusion protein of SEQ ID NO:56 without the signal sequence can be:

GFGISGRHSI TVTTVASAGN IGEDGILSCT FEPDIKLSDI
VIQWLKEGVL GLVHEFKEGK DELSEQDEMF RGRTAVFADQ
VIVGNASLRL KNVQLTDAGT YKCYIITSKG KGNANLEYKT
GAFSMPEVNV DYNASSETLR CEAPRWFPQP TVVWASQVDQ
GANFSEVSNT SFELNSENVT MKVVSVLYNV TINNTYSCMI
ENDIAKATGD IKVTESEIKR RSEPKSCDKT HTCPPCPAPE
LLGGPSVFLF PPKPKDTLMI SRTPEVTCVV VDVSHEDPEV
KFNWYVDGVE VHNAKTKPRE EQYQSTYRVV SVLTVLHQDW
LNGKEYKCKV SNKALPAPIE KTISKAKGQP REPQVYTLPP
SRDELTKNQV SLTCLVKGFY PSDIAVEWES NGQPENNYKT
TPPVLDSDGS FFLYSKLTVD KSRWQQGNVF SCSVMHEALH
NHYTQKSLSL SPGK

### (SEQ ID NO:57).

Changes in SEQ ID NO:56 relative to SEQ ID NO:22 are illustrated below:

MEWSWVFLFFLSVTTGVHSGFGISGRHSITVTTVASAGNIGEDGILSCTF
EPDIKLSDIVIQWLKEGVLGLVHEFKEGKDELSEQDEMFRGRTAVFADQV
IVGNASLRLKNVQLTDAGTYKCYIITSKGKGNANLEYKTGAFSMPEVNVD
YNASSETLRCEAPRWFPQPTVVWASQVDQGANFSEVSNTSFELNSENVTM
KVVSVLYNVTINNTYSCMIENDIAKATGDIKVTESEIKRRSEPKSCDKTH
TCPPCPAPELLGGPSVFLFPPKPKDTLMISRTPEVTCVVVDVSHEDPEVK
FNWYVDGVEVHNAKTKPREEQY [symbol] STYRVVSVLTVLHQDWLNGKEYKCKVS
NKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFS
CSVMHEALHNHYTQKSLSLSPGK

### (SEQ ID NO:65).

In another embodiment, a representative human B7-H4 fusion protein has at least 80%, 85%, 90%, 95%, 99% or 100% sequence identity to:

MEWSWVFLFF LSVTTGVHSF GISGRHSITV TTVASAGNIG
EDGILSCTFE PDIKLSDIVI QWLKEGVLGL VHEFKEGKDE
LSEQDEMFRG RTAVFADQVI VGNASLRLKN VQLTDAGTYK
CYIITSKGKG NANLEYKTGA FSMPEVNVDY NASSETLRCE
APRWFPQPTV VWASQVDQGA NFSEVSNTSF ELNSENVTMK
VVSVLYNVTI NNTYSCMIEN DIAKATGDIK VTESEIKQQS
HLQLLNSKAS EPKSSDKTHT CPPCPAPELL GGPSVFLFPP
KPKDTLMISR TPEVTCVVVD VSHEDPEVKF NWYVDGVEVH
NAKTKPREEQ YNSTYRVVSV LTVLHQDWLN GKEYKCKVSN
KALPAPIEKT ISKAKGQPRE PQVYTLPPSR DELTKNQVSL
TCLVKGFYPS DIAVEWESNG QPENNYKTTP PVLDSDGSFF
LYSKLTVDKS RWQQGNVFSC SVMHEALHNH YTQKSLSLSP
G

### (SEQ ID NO:58).

The amino acid sequence of the human B7-H4 fusion protein of SEQ ID NO:58 without the signal sequence can be:

FGISGRHSIT VTTVASAGNI GEDGILSCTF EPDIKLSDIV
IQWLKEGVLG LVHEFKEGKD ELSEQDEMFR GRTAVFADQV
IVGNASLRLK NVQLTDAGTY KCYIITSKGK GNANLEYKTG
AFSMPEVNVD YNASSETLRC EAPRWFPQPT VVWASQVDQG
ANFSEVSNTS FELNSENVTM KVVSVLYNVT INNTYSCMIE
NDIAKATGDI KVTESEIKQQ SHLQLLNSKA SEPKSSDKTH
TCPPCPAPEL LGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS RDELTKNQVS LTCLVKGFYP SDIAVEWESN
GQPENNYKTT PPVLDSDGSF FLYSKLTVDK SRWQQGNVFS
CSVMHEALHN HYTQKSLSLS PG

### (SEQ ID NO:59).

Changes in SEQ ID NO:58 relative to SEQ ID NO:22 are illustrated below:

MEWSWVFLFFLSVTTGVHS [symbol] FGISGRHSITVTTVASAGNIGEDGI [symbol] SCTF
EPDIKLSDIVIQWLKEGVLGLVHEFKEGKDELSEQDEMFRGRTAVFADQV
IVGNASLRLKNVQLTDAGTYKCYIITSKGKGNANLEYKTGAFSMPEVNVD
YNASSETLRCEAPRWFPQPTVVWASQVDQGANFSEVSNTSFELNSENVTM
KVVSVLYNVTINNTYSCMIENDIAKATGDIKVTESEIK [symbol] SHLQLLNSKAS
EPKS [symbol] DKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

[symbol]

### (SEQ ID NO:74).

In another embodiment, a representative human B7-H4 fusion protein has at least 80%, 85%, 90%, 95%, 99% or 100% sequence identity to:

MEWSWVFLFF LSVTTGVHSF GISGRHSITV TTVASAGNIG
EDGILSCTFE PDIKLSDIVI QWLKEGVLGL VHEFKEGKDE
LSEQDEMFRG RTAVFADQVI VGNASLRLKN VQLTDAGTYK
CYIITSKGKG NANLEYKTGA FSMPEVNVDY NASSETLRCE
APRWFPQPTV VWASQVDQGA NFSEVSNTSF ELNSENVTMK
VVSVLYNVTI NNTYSCMIEN DIAKATGDIK VTESEIKRRS
HLQLLNSKAS ESKYGPPCPP CPAPEFLGGP SVFLFPPKPK
DTLMISRTPE VTCVVVDVSQ EDPEVQFNWY VDGVEVHNAK
TKPREEQFNS TYRVVSVLTV LHQDWLNGKE YKCKVSNKGL
PSSIEKTISK AKGQPREPQV YTLPPSQEEM TKNQVSLTCL
VKGFYPSDIA VEWESNGQPE NNYKTTPPVL DSDGSFFLYS
RLTVDKSRWQ EGNVFSCSVM HEALHNHYTQ KSLSLSPG

### (SEQ ID NO:32).

The amino acid sequence of the human B7-H4 fusion protein of SEQ ID NO:32 without the signal sequence can be:

FGISGRHSIT VTTVASAGNI GEDGILSCTF EPDIKLSDIV
IQWLKEGVLG LVHEFKEGKD ELSEQDEMFR GRTAVFADQV
IVGNASLRLK NVQLTDAGTY KCYIITSKGK GNANLEYKTG
AFSMPEVNVD YNASSETLRC EAPRWFPQPT VVWASQVDQG
ANFSEVSNTS FELNSENVTM KVVSVLYNVT INNTYSCMIE
NDIAKATGDI KVTESEIKRR SHLQLLNSKA SESKYGPPCP
PCPAPEFLGG PSVFLFPPKP KDTLMISRTP EVTCVVVDVS
QEDPEVQFNW YVDGVEVHNA KTKPREEQFN STYRVVSVLT
VLHQDWLNGK EYKCKVSNKG LPSSIEKTIS KAKGQPREPQ
VYTLPPSQEE MTKNQVSLTC LVKGFYPSDI AVEWESNGQP
ENNYKTTPPV LDSDGSFFLY SRLTVDKSRW QEGNVFSCSV
MHEALHNHYT QKSLSLSPG

### (SEQ ID NO:33).

Changes in SEQ ID NO: 32 relative to SEQ ID NO:22 are illustrated below:

MEWSWVFLFFLSVTTGVHS [symbol] FGISGRHSITVTTVASAGNIGEDGI [symbol] SCTF
EPDIKLSDIVIQWLKEGVLGLVHEFKEGKDELSEQDEMFRGRTAVFADQV
IVGNASLRLKNVQLTDAGTYKCYIITSKGKGNANLEYKTGAFSMPEVNVD
YNASSETLRCEAPRWFPQPTVVWASQVDQGANFSEVSNTSFELNSENVTM
KVVSVLYNVTINNTYSCMIENDIAKATGDIKVTESEIKRRS [symbol]
E [symbol] K [symbol] CPPC
PAPE [symbol] LGGPSVFLFPPKPKDTLMISRTPEVTCVVVDVS [symbol] EDPEV [symbol] FNWYV
DGVEVHNAKTKPREEQ [symbol] NSTYRVVSVLTVLHQDWLNGKEYKCKVSNK [symbol] LP [symbol]
IEKTISKAKGQPREPQVYTLPPS [symbol] E [symbol] TKNQVSLTCLVKGFYPSDIAVEW
ESNGQPENNYKTTPPVLDSDGSFFLYS [symbol] LTVDKSRWQ [symbol] GNVFSCSVMHEA
LHNHYTQKSLSLSPG

[symbol]

(SEQ ID NO:73). Amino acid sequences in strike-through are deleted.

Binding between B7-H4 and its receptors can be mediated by the IgV domain. Therefore, in some embodiments, the fusion proteins include a B7-H4 polypeptide having an IgV domain, but wherein part or all of the IgC is absent. For example, in some embodiments, the fusion protein includes a part of the B7-H4 extracellular domain, but does not include all or part of SEQ ID NOS: 12, 13, 14, 15, 16, or peptide having 80%, 85%, 90%, 95%, 99% identity to SEQ ID NOS: 12, 13, 14, 15, or 16. In some embodiments, the fusion protein includes SEQ ID NOS:8, 9, 10, or 11.

Therefore, in another embodiment, a representative human B7-H4 fusion protein has at least 80%, 85%, 90%, 95%, 99% or 100% sequence identity to:

MEWSWVFLFF LSVTTGVHSF GISGRHSITV TTVASAGNIG
EDGILSCTFE PDIKLSDIVI QWLKEGVLGL VHEFKEGKDE
LSEQDEMFRG RTAVFADQVI VGNASLRLKN VQLTDAGTYK
CYIITSKGKG NANLEYKTGA EPKSSDKTHT CPPCPAPELL
GGPSVFLFPP KPKDTLMISR TPEVTCVVVD VSHEDPEVKF
NWYVDGVEVH NAKTKPREEQ YNSTYRVVSV LTVLHQDWLN
GKEYKCKVSN KALPAPIEKT ISKAKGQPRE PQVYTLPPSR
DELTKNQVSL TCLVKGFYPS DIAVEWESNG QPENNYKTTP
PVLDSDGSFF LYSKLTVDKS RWQQGNVFSC SVMHEALHNH
YTQKSLSLSP G

### (SEQ ID NO:26).

The amino acid sequence of the human B7-H4 fusion protein of SEQ ID NO:26 without the signal sequence can be:

FGISGRHSIT VTTVASAGNI GEDGILSCTF EPDIKLSDIV
IQWLKEGVLG LVHEFKEGKD ELSEQDEMFR GRTAVFADQV
IVGNASLRLK NVQLTDAGTY KCYIITSKGK GNANLEYKTG
AEPKSSDKTH TCPPCPAPEL LGGPSVFLFP PKPKDTLMIS
RTPEVTCVVV DVSHEDPEVK FNWYVDGVEV HNAKTKPREE
QYNSTYRVVS VLTVLHQDWL NGKEYKCKVS NKALPAPIEK
TISKAKGQPR EPQVYTLPPS RDELTKNQVS LTCLVKGFYP
SDIAVEWESN GQPENNYKTT PPVLDSDGSF FLYSKLTVDK
SRWQQGNVFS CSVMHEALHN HYTQKSLSLS PG

### (SEQ ID NO:27).

Changes in SEQ ID NO:26 relative to SEQ ID NO:22 are illustrated below:

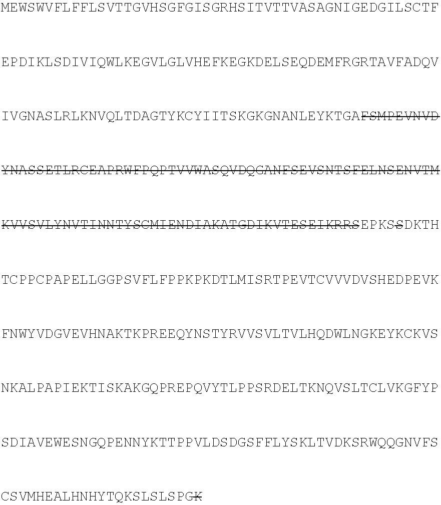

(SEQ ID NO:69). Amino acid sequences in strike-through are deleted.

In another embodiment, a representative human B7-H4 fusion protein has at least 80%, 85%, 90%, 95%, 99% or 100% sequence identity to:

MEWSWVFLFF LSVTTGVHSF GISGRHSITV TTVASAGNIG
EDGILSCIFE PDIKLSDIVI QWLKEGVLGL VHEFKEGKDE
LSEQDEMFRG RTAVFADQVI VGNASLRLKN VQLTDAGTYK
CYIITSKGKG NANLEYKTGA FSMPEEPKSS DKTHTCPPCP
APELLGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSRDELTK NQVSLTCLVK GFYPSDIAVE WESNGQPENN
YKTTPPVLDS DGSFFLYSKL TVDKSRWQQG NVFSCSVMHE
ALHNHYTQKS LSLSPG

### (SEQ ID NO:28).

The amino acid sequence of the human B7-H4 fusion protein of SEQ ID NO:28 without the signal sequence can be:

FGISGRHSIT VITVASAGNI GEDGILSCTF EPDIKLSDIV
IQWLKEGVLG LVHEFKEGKD ELSEQDEMFR GRTAVFADQV
IVGNASLRLK NVQLTDAGTY KCYIITSKGK GNANLEYKTG
AFSMPEEPKS SDKTHTCPPC PAPELLGGPS VFLFPPKPKD
TLMISRTPEV TCVVVDVSHE DPEVKFNWYV DGVEVHNAKT
KPREEQYNST YRVVSVLTVL HQDWLNGKEY KCKVSNKALP
APIEKTISKA KGQPREPQVY TLPPSRDELT KNQVSLTCLV
KGFYPSDIAV EWESNGQPEN NYKTTPPVLD SDGSFFLYSK
LTVDKSRWQQ GNVFSCSVMH EALHNHYTQK SLSLSPG

### (SEQ ID NO:29).

Changes in SEQ ID NO:28 relative to SEQ ID NO:22 are illustrated below:

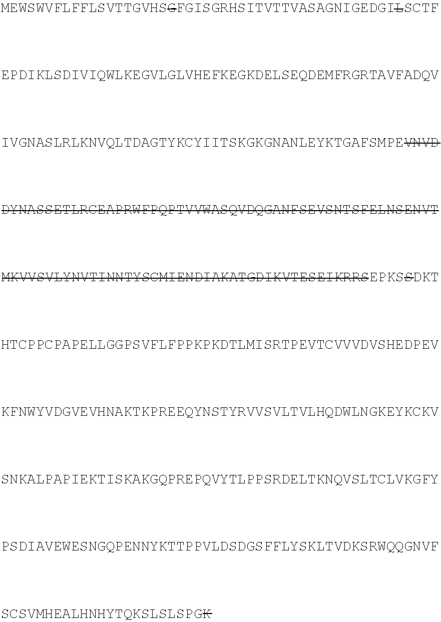

(SEQ ID NO:70). Amino acid sequences in strike-through are deleted.

In another embodiment, a representative human B7-H4 fusion protein has at least 80%, 85%, 90%, 95%, 99% or 100% sequence identity to:

MEWSWVFLFF LSVTTGVHSF GISGRHSITV TTVASAGNIG
EDGILSCTFE PDIKLSDIVI QWLKEGVLGL VHEFKEGKDE
LSEQDEMFRG RTAVFADQVI VGNASLRLKN VQLTDAGTYK
CYIITSKGKG NANLEYKTGA FSMPEVNVDE PKSSDKTHTC
PPCPAPELLG GPSVFLFPPK PKDTLMISRT PEVTCVVVDV
SHEDPEVKFN WYVDGVEVHN AKTKPREEQY NSTYRVVSVL
TVLHQDWLNG KEYKCKVSNK ALPAPIEKTI SKAKGQPREP
QVYTLPPSRD ELTKNQVSLT CLVKGFYPSD IAVEWESNGQ
PENNYKTTPP VLDSDGSFFL YSKLTVDKSR WQQGNVFSCS
VMHEALHNHY TQKSLSLSPG

### (SEQ ID NO:30).

The amino acid sequence of the human B7-H4 fusion protein of SEQ ID NO:30 without the signal sequence can be:

FGISGRHSIT VTTVASAGNI GEDGILSCTF EPDIKLSDIV
IQWLKEGVLG LVHEFKEGKD ELSEQDEMFR GRTAVFADQV
IVGNASLRLK NVQLTDAGTY KCYIITSKGK GNANLEYKTG
AFSMPEVNVD EPKSSDKTHT CPPCPAPELL GGPSVFLFPP
KPKDTLMISR TPEVTCVVVD VSHEDPEVKF NWYVDGVEVH
NAKTKPREEQ YNSTYRVVSV LTVLHQDWLN GKEYKCKVSN
KALPAPIEKT ISKAKGQPRE PQVYTLPPSR DELTKNQVSL
TCLVKGFYPS DIAVEWESNG QPENNYKTTP PVLDSDGSFF
LYSKLTVDKS RWQQGNVFSC SVMHEALHNH YTQKSLSLSP G

### (SEQ ID NO:31).

Changes in SEQ ID NO:30 relative to SEQ ID NO:22 are illustrated below:

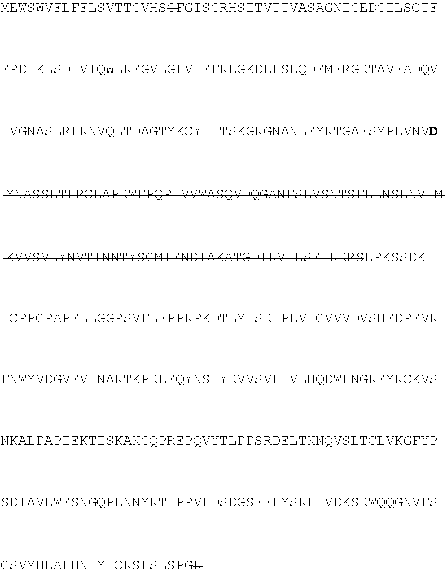

(SEQ ID NO:71). Amino acid sequences in strike-through are deleted.

Binding between B7-H4 and its receptors can be mediated by the IgC domain. Therefore, in some embodiments, the fusion proteins include a B7-H4 polypeptide having an IgC domain, but wherein part or all of the IgV is absent. For example, in some embodiments, the fusion protein includes a part of the B7-H4 extracellular domain, but does not include all or part of SEQ ID NOS:8, 9, 10, or 11. In some embodiments, the fusion protein includes SEQ ID NOS: 12, 13, 14, 15, 16, or peptide having 80%, 85%, 90%, 95%, 99% identity to SEQ ID NOS: 12, 13, 14, 15, or 16.

The fusion protein can include, for example,

EVNVDYNASS ETLRCEAPRW FPQPTVVWAS QVDQGANFSE
VSNTSFELNS ENVTMKVVSV LYNVTINNTY SCMIENDIAK
ATGDIKVTES EIKQQSHLQL LNSKAS

(SEQ ID NO:19) or peptide having 80%, 85%, 90%, 95%, 99% identity to SEQ ID NOS: 12, 13, 14, 15, or 16.

An exemplary B7-H4-Ig fusion protein including an IgC domain and without an IgV domain can have at least 80%, 85%, 90%, 95%, 99% or 100% sequence identity to:

MEWSWVFLFF LSVTTGVHSE VNVDYNASSE TLRCEAPRWF
PQPTVVWASQ VDQGANFSEV SNTSFELNSE NVTMKVVSVL
YNVTINNTYS CMIENDIAKA TGDIKVTESE IKQQSHLQLL
NSKASEPKSS DKTHTCPPCP APELLGGPSV FLFPPKPKDT
LMISRTPEVT CVVVDVSHED PEVKFNWYVD GVEVHNAKTK
PREEQYNSTY RVVSVLTVLH QDWLNGKEYK CKVSNKALPA
PIEKTISKAK GQPREPQVYT LPPSRDELTK NQVSLTCLVK
GFYPSDIAVE WESNGQPENN YKTTPPVLDS DGSFFLYSKL
TVDKSRWQQG NVFSCSVMHE ALHNHYTQKS LSLSPG

### (SEQ ID NO:20).

The amino acid sequence of the fusion protein of SEQ ID NO:20 without the signal sequence can be:

EVNVDYNASS ETLRCEAPRW FPQPTVVWAS QVDQGANFSE
VSNTSFELNS ENVTMKVVSV LYNVTINNTY SCMIENDIAK
ATGDIKVTES EIKQQSHLQL LNSKASEPKS SDKTHTCPPC
PAPELLGGPS VFLFPPKPKD TLMISRTPEV TCVVVDVSHE
DPEVKFNWYV DGVEVHNAKT KPREEQYNST YRVVSVLTVL
HQDWLNGKEY KCKVSNKALP APIEKTISKA KGQPREPQVY
TLPPSRDELT KNQVSLTCLV KGFYPSDIAV EWESNGQPEN
NYKTTPPVLD SDGSFFLYSK LTVDKSRWQQ GNVFSCSVMH
EALHNHYTQK SLSLSPG

### (SEQ ID NO:21).

Changes in SEQ ID NO:20 relative to SEQ ID NO:22 are illustrated below:

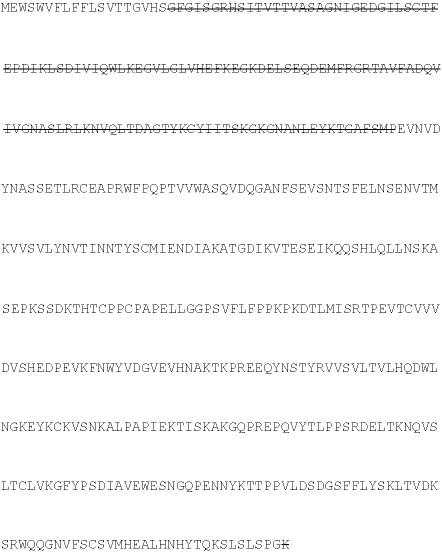

(SEQ ID NO:72). Amino acid sequences in strike-through are deleted.

Another exemplary B7-H4-Ig fusion protein including an IgC domain and without an IgV domain can have at least 80%, 85%, 90%, 95%, 99% or 100% sequence identity to:

MEWSWVFLFF LSVTTGVHSE VNVDYNASSE TLRCEAPRWF
PQPTVVWASQ VDQGANFSEV SNTSFELNSE NVTMKVVSVL
YNVTINNTYS CMIENDIAKA TGDIKVTESE IKSEPKSDKT
HTCPPCPAPE LLGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SRDELTKNQV SLTCLVKGFY PSDIAVEWES
NGQPENNYKT TPPVLDSDGS FFLYSKLTVD KSRWQQGNVF
SCSVMHEALH NHYTQKSLSL SPG

### (SEQ ID NO:60).

The amino acid sequence of the fusion protein of SEQ ID NO:20 without the signal sequence can be:

EVNVDYNASS ETLRCEAPRW FPQPTVVWAS QVDQGANFSE
VSNTSFELNS ENVTMKVVSV LYNVTINNTY SCMIENDIAK
ATGDIKVTES EIKSEPKSDK THTCPPCPAP ELLGGPSVFL
FPPKPKDTLM ISRTPEVTCV VVDVSHEDPE VKFNWYVDGV
EVHNAKTKPR EEQYNSTYRV VSVLTVLHQD WLNGKEYKCK
VSNKALPAPI EKTISKAKGQ PREPQVYTLP PSRDELTKNQ
VSLTCLVKGF YPSDIAVEWE SNGQPENNYK TTPPVLDSDG
SFFLYSKLTV DKSRWQQGNV FSCSVMHEAL HNHYTQKSLS
LSPG

### (SEQ ID NO:61).

A murine B7-H4-Ig can have the sequence

MEWSWVFLFF LSVTTGVHSG FGISGKHFIT VTTFTSAGNI
GEDGTLSCTF EPDIKLNGIV IQWLKEGIKG LVHEFKEGKD
DLSQQHEMFR GRTAVFADQV VVGNASLRLK NVQLTDAGTY
TCYIRSSKGK GNANLEYKTG AFSMPEINVD YNASSESLRC
EAPRWFPQPT VAWASQVDQG ANFSEVSNTS FELNSENVTM
KVVSVLYNVT INNTYSCMIE NDIAKATGDI KVTDSEVKRR
SQLQLLNSGE PRGPTIKPCP PCKCPAPNLL GGPSVFIFPP
KIKDVLMISL SPIVTCVVVD VSEDDPDVQI SWFVNNVEVH
TAQTQTHRED YNSTLRVVSA LPIQHQDWMS GKEFKCKVNN
KDLPAPIERT ISKPKGSVRA PQVYVLPPPE EEMTKKQVTL
TCMVTDFMPE DIYVEWTNNG KTELNYKNTE PVLDSDGSYF
MYSKLRVEKK NWVERNSYSC SVVHEGLHNH HTTKSFSRTP
GK

### (SEQ ID NO:34).

SEQ ID NO:34 without the signal sequence is

GFGISGKHFI TVTTFTSAGN IGEDGTLSCT FEPDIKLNGI
VIQWLKEGIK GLVHEFKEGK DDLSQQHEMF RGRTAVFADQ
VVVGNASLRL KNVQLTDAGT YTCYIRSSKG KGNANLEYKT
GAFSMPEINV DYNASSESLR CEAPRWFPQP TVAWASQVDQ
GANFSEVSNT SFELNSENVT MKVVSVLYNV TINNTYSCMI
ENDIAKATGD IKVTDSEVKR RSQLQLLNSG EPRGPTIKPC
PPCKCPAPNL LGGPSVFIFP PKIKDVLMIS LSPIVTCVVV
DVSEDDPDVQ ISWFVNNVEV HTAQTQTHRE DYNSTLRVVS
ALPIQHQDWM SGKEFKCKVN NKDLPAPIER TISKPKGSVR
APQVYVLPPP EEEMTKKQVT LTCMVTDFMP EDIYVEWTNN
GKTELNYKNT EPVLDSDGSY FMYSKLRVEK KNWVERNSYS
CSVVHEGLHN HHTTKSFSRT PGK

### (SEQ ID NO:35).

SEQ ID NO:34 can be encoded by the sequence

| | |
|---|---|
| ATGGAGTGGT CATGGGTTTT TCTGTTCTTT CTTAGCGTGA CTACAGGCGT CCATTCAGGA | 60 |
| TTCGGCATAA GCGGCAAGCA CTTCATCACA GTTACAACGT TTACAAGTGC GGGGAACATT | 120 |
| GGGGAAGATG GAACATTGTC ATGTACATTT GAGCCAGATA TCAAACTCAA TGGAATAGTA | 180 |
| ATTCAGTGGC TTAAGGAGGG CATCAAGGGC CTGGTCCACG AATTTAAGGA GGGGAAAGAC | 240 |
| GATCTGTCTC AGCAGCACGA GATGTTCAGG GGCAGAACCG CCGTCTTCGC AGACCAGGTT | 300 |
| GTGGTAGGCA ACGCCAGTTT GCGGCTGAAA AACGTGCAGC TGACTGACGC CGGCACCTAC | 360 |
| ACATGCTATA TCCGGTCCTC TAAGGGCAAG GGGAACGCTA ATCTCGAGTA CAAAACAGGC | 420 |
| GCCTTTTCTA TGCCAGAGAT CAACGTGGAC TATAACGCAA GCTCTGAAAG TCTGAGATGC | 480 |
| GAGGCGCCAA GGTGGTTCCC TCAGCCCACC GTCGCGTGGG CTTCCCAGGT GGATCAAGGC | 540 |
| GCCAACTTTT CTGAGGTTTC TAACACCAGC TTCGAACTGA ACAGCGAAAA TGTGACAATG | 600 |
| AAGGTAGTCA GCGTTCTGTA TAACGTGACC ATCAACAATA CTTACTCCTG TATGATAGAA | 660 |
| AATGATATAG CCAAGGCTAC AGGAGATATT AAAGTGACGG ATTCAGAAGT GAAAAGGAGG | 720 |
| AGTCAACTGC AACTCTTGAA TAGCGGCGAG CCAAGAGGTC CTACGATCAA GCCCTGCCCG | 780 |
| CCTTGTAAAT GCCCAGCTCC AAATTTGCTG GGTGGACCGT CAGTCTTTAT CTTCCCGCCA | 840 |
| AAGATAAAGG ACGTCTTGAT GATTAGTCTG AGCCCCATCG TGACATGCGT TGTGGTGGAT | 900 |
| GTTTCAGAGG ATGACCCCGA CGTGCAAATC AGTTGGTTCG TTAACAACGT GGAGGTGCAT | 960 |
| ACCGCTCAAA CCCAGACCCA CAGAGAGGAT TATAACAGCA CCCTGCGGGT AGTGTCCGCC | 1020 |
| CTGCCGATCC AGCATCAGGA TTGGATGAGC GGGAAAGAGT TCAAGTGTAA GGTAAACAAC | 1080 |
| AAAGATCTGC CAGCGCCGAT TGAACGAACC ATTAGCAAGC CGAAAGGGAG CGTGCGCGCA | 1140 |
| CCTCAGGTTT ACGTCCTTCC TCCACCAGAA GAGGAGATGA CGAAAAAGCA GGTGACCCTG | 1200 |
| ACATGCATGG TAACTGACTT TATGCCAGAA GATATTTACG TGGAATGGAC TAATAACGGA | 1260 |
| AAGACAGAGC TCAATTACAA GAACACTGAG CCTGTTCTGG ATTCTGATGG CAGCTACTTT | 1320 |
| ATGTACTCCA AATTGAGGGT CGAGAAGAAG AATTGGGTCG AGAGAAACAG TTATAGTTGC | 1380 |
| TCAGTGGTGC ATGAGGGCCT CCATAATCAT CACACCACAA AGTCCTTCAG CCGAACGCCC | 1440 |
| GGGAAATGA | 1449 |

### (SEQ ID NO:36).

The aforementioned exemplary fusion proteins can incorporate any combination of the variants described herein. In another embodiment the terminal lysine of the aforementioned exemplary fusion proteins is deleted.

The disclosed fusion proteins can be isolated using standard molecular biology techniques. For example, an expression vector containing a DNA sequence encoding a B7-H4-Ig fusion protein is transfected into 293 cells by calcium phosphate precipitation and cultured in serum-free DMEM. The supernatant is collected at 72 h and the fusion protein is purified by Protein G, or preferably Protein A SEPHAROSE® columns (Pharmacia, Uppsala, Sweden).

B. Antibodies

In some embodiments, B7-H4 receptor agonists are antibodies. Methods of producing antibodies are well known and within the ability of one of ordinary skill in the art and are described in more detail below.

The B7-H4 receptor agonistic antibodies disclosed herein specifically bind to a B7-H4 receptor and are capable of activating the B7-H4 receptor to effect signaling inside the cell expressing the B7-H4 receptor. Agonistic B7-H4 receptor antibodies thus mimic the effects of natural ligands for B7-H4 receptors, including B7-H4. These antibodies are defined as “activating” or “agonistic” antibodies. In preferred embodiments the agonistic antibodies specifically bind to a portion of the extracellular domain of B7-H4 receptors.

The antibody can be an antibody or an antigen binding fragment thereof that binds to a neuropilin, a plexin, or a complex thereof.

The antibody can be an anti-B7-H4 antibody or an anti-semphorin antibody. For example, in some embodiments, the antibody is increases, enhances, or stabilizes interaction between B7-H4 and/or semaphorin with the receptor complex.

In another embodiment, the antibody can target an alternative ligand or co-ligand for a neuropilin or a plexin such as VEGF, or a semaphorin that is not a co-ligand with B7-H4, to encourage binding of neuropilin to B7-H4 or a fusion protein thereof.

Bi-specific antibodies specific for two or more B7-H4 receptor epitopes; or two or more co-receptor epitopes; or two or more B7-H4 receptor ligand epitopes; or a ligand epitope and a receptor epitope can be generated using methods generally known in the art. Homodimeric antibodies can also be generated by cross-linking techniques known in the art (e.g., Wolff et al., Cancer Res. 53: 2560-2565).

In some embodiments, one or more agonist antibodies are administered in combination.

C. Semaphorins

The Examples below show that semaphorins such as SEMA3A can increase B7-H4 binding to neuropilins such as NRP-1. Accordingly, in some embodiments, the B7-H4 receptor agonist is a semaphorin polypeptide, or a functional fragment or variant thereof, or fusion protein thereof. In the most preferred embodiments, a semaphorin is co-administered with another B7-H4 agonist, for example a B7-H4 fusion protein. In preferred embodiments, the semaphorin is a soluble semaphorin, or example a secreted semaphorin or a soluble fragment of membrane semaphorin. Preferably the semaphorin can bind to B7-H4, to a neuropilin, to a plexin, or a combination thereof. Semaphorins and sequences thereof, as well as fragments and variants thereof, or disclosed above.

Semaphorin fusion proteins are also provided. Fusion proteins are discussed above with reference to B7-H4. For example, semaphorin fusion proteins can have a first fusion partner including all or a part of a semaphorin protein fused to a second polypeptide directly or via a linker peptide sequence that is fused to the second polypeptide. The fusion proteins optionally contain a domain that functions to dimerize or multimerize two or more fusion proteins. The peptide/polypeptide linker domain can either be a separate domain, or alternatively can be contained within one of the other domains (semaphorin polypeptide or second polypeptide) of the fusion protein. Similarly, the domain that functions to dimerize or multimerize the fusion proteins can either be a separate domain, or alternatively can be contained within one of the other domains (semaphorin polypeptide, second polypeptide or peptide/polypeptide linker domain) of the fusion protein. In one embodiment, the dimerization/multimerization domain and the peptide/polypeptide linker domain are the same.

Fusion proteins disclosed herein are of formula I:

N—R₁—R₂—R₃—C

wherein “N” represents the N-terminus of the fusion protein, “C” represents the C-terminus of the fusion protein. In the preferred embodiment, “R₁” is a semaphorin polypeptide, “R₂” is an optional peptide/polypeptide linker domain, and “R₃” is a second polypeptide. Alternatively, R₃ may be a semaphorin polypeptide and R₁ may be a second polypeptide.

Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric. Dimerization and multimerization domains are discussed in detail above with respect to semaphorin receptor fusions proteins.

In one embodiment, the first fusion partner is a fragment or variant of semaphorin, such as those discussed above. In a preferred embodiment, the fusion protein includes a secreted semaphorin or a soluble fragment of membrane semaphorin, or a functional fragment or variant thereof fused to an Ig Fc region. Recombinant semaphorin fusion proteins can be prepared by fusing the coding region of the semaphorin or a fragment thereof to the Fc region of human IgG1 or mouse IgG2a, or other suitable Ig domain, as described in more detail above with respect to B7-H4 and B7-H4 receptor fusion proteins.

In a particular embodiment, the semaphorin is SEQ ID:62, or a functional fragment or variant thereof.

### V. Pharmaceutical Compositions

Pharmaceutical compositions including B7-H4 receptor agonists or antagonists may be administered by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration. Compositions containing agonists or antagonists of B7-H4 receptors can additionally be formulated for enteral administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected. Typically, therapeutically effective amounts of B7-H4 receptor agonists cause an immune inhibitory response to be activated or sustained or an immune stimulatory response to be reduced or inhibited whereas therapeutically effective amounts of B7-H4 receptor antagonists cause an immune stimulatory response to be activated or sustained or an immune inhibitory response to be reduced or inhibited.

The selected dosage depends upon the desired therapeutic effect, on the route of administration, and on the duration of the treatment desired. Generally dosage levels of 0.001 to 20 mg/kg of body weight daily are administered to mammals. Generally, for intravenous injection or infusion, dosage may be lower.

B7-H4 receptor agonists or antagonists can bind directly to the B7-H4 receptor. B7-H4 receptor antagonists can bind to B7-H4. Methods for measuring binding affinity between two molecules are well known in the art, and include, but are not limited to, fluorescence activated cell sorting (FACS), surface plasmon resonance, fluorescence anisotropy, affinity chromatography and affinity selection-mass spectrometry.

Activities of B7-H4 receptors that can be measured include effects on T cell survival, T cell activation, T cell proliferation, Treg survival, Treg activation, Treg proliferation, Treg differentiation, cytokine release, and the activation or inhibition of various protein kinase signaling pathways and transcriptional factors. Effect of B7-H4 receptor agonists or antagonists on inhibiting or reducing T cell activation can be measured as a decrease in proliferation or secretion of cytokines, including, but not limited to, IL-2. Methods for measuring cell survival, cell proliferation, protein phosphorylation, activation of various transcriptional factors including NF-κB, JNK, and AP-1, and cytokine secretion are well known to those of skill in the art.

Accordingly, compositions, dosages and treatments and the activities and effects thereof can be compared to a control, for example, an untreated subject or to the subject prior to treatment. Disclosed activities suitable for comparison to a control and assays for making the measurements are disclosed herein and include the assays described in the Examples below.

A. Formulations for Parenteral Administration

In a preferred embodiment, the disclosed compositions, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include sterile water, buffered saline (e.g., Tris-HCl, acetate, phosphate), pH and ionic strength; and optionally, additives such as detergents and solubilizing agents (e.g., TWEEN® 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

B. Controlled Delivery Polymeric Matrices

Compositions containing one or more B7-H4 receptor agonists or antagonists can be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel. The matrix can also be incorporated into or onto a medical device to modulate an immune response, to prevent infection in an immunocompromised patient (such as an elderly person in which a catheter has been inserted or a premature child) or to aid in healing, as in the case of a matrix used to facilitate healing of pressure sores, decubitis ulcers, etc.

Either non-biodegradable or biodegradable matrices can be used for delivery of B7-H4 agonists or antagonists, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

Controlled release oral formulations may be desirable. B7-H4 agonists or antagonists can be incorporated into an inert matrix which permits release by either diffusion or leaching mechanisms, e.g., films or gums. Slowly disintegrating matrices may also be incorporated into the formulation. Another form of a controlled release is one in which the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the active agent (or derivative) or by release of the active agent beyond the stomach environment, such as in the intestine. To ensure full gastric resistance an enteric coating (i.e., impermeable to at least pH 5.0) is essential. These coatings may be used as mixed films or as capsules such as those available from Banner Pharmacaps.

The devices can be formulated for local release to treat the area of implantation or injection and typically deliver a dosage that is much less than the dosage for treatment of an entire body. The devices can also be formulated for systemic delivery. These can be implanted or injected subcutaneously.

C. Formulations for Enteral Administration

B7-H4 receptor agonists or antagonists can also be formulated for oral delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 21st Ed. (2005, Lippincott, Williams & Wilins, Baltimore, Md. 21201) pages 889-964. The compositions may be prepared in liquid form, or may be in dried powder (e.g., lyophilized) form. Liposomal or polymeric encapsulation may be used to formulate the compositions. See also Marshall, K. In: Modern Pharmaceutics Edited by G. S. Banker and C. T. Rhodes Chapter 10, 1979. In general, the formulation will include the active agent and inert ingredients which protect the B7-H4 receptor agonists or antagonists in the stomach environment, and release of the biologically active material in the intestine.

Liquid dosage forms for oral administration, including pharmaceutically acceptable emulsions, solutions, suspensions, and syrups, may contain other components including inert diluents; adjuvants such as wetting agents, emulsifying and suspending agents; and sweetening, flavoring, and perfuming agents.

D. Vaccine Formulations Including B7-H4 Receptor Antagonists

Vaccines require strong T cell response to eliminate cancer cells and infected cells. B7-H4 receptor antagonists can be administered as a component of a vaccine to inhibit or reduce inhibition of T cells by endogenous B7-H4. Vaccines disclosed herein include antigens, a source of B7-H4 receptor antagonists, and optionally adjuvants.

1. Antigens

As used herein, an antigen is an entity to which an antibody specifically binds. Antigens can be any substance that evokes an immunological response in a subject. Representative antigens include peptides, proteins, polysaccharides, saccharides, lipids, nucleic acids, or combinations thereof. The antigen can be derived from a tumor or from a transformed cell such as a cancer or leukemic cell and can be a whole cell or immunogenic component thereof, e.g., cell wall components or molecular components thereof.

Suitable antigens are known in the art and are available from commercial government and scientific sources. The antigens may be purified or partially purified polypeptides derived from tumors or other sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

2. Viral Antigens

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

3. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

4. Parasitic Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

5. Tumor Antigens

The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAAO205, Mart2, Mum-1, 2, and 3, neo-PAP, myosin class I, OS-9, pm1-RARα fusion protein, PTPRK, K-ras, N-ras, Triosephosphate isomeras, Bage-1, Gage 3,4,5,6,7, GnTV, Herv-K-me1, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

6. Adjuvants

Optionally, the vaccines described herein may include one or more adjuvants. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminum salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor. Additional adjuvants can include polypeptides, including polypeptides of the B7 family. Such proteinaceous adjuvants may be provided as the full-length polypeptide or an active fragment thereof, or in the form of DNA, such as plasmid DNA.

### VI. Screening for Receptor Agonists and Antagonists

B7-H4 receptors, nucleic acids encoding B7-H4 receptor polypeptides, antibodies that bind to B7-H4 receptors, and agonists and antagonists of B7-H4 receptors are useful as research tools for in vitro and in vivo studies of T cell and immune system function. For example, these compositions can be used to measure B7-H4/B7-H4 receptor interactions, for the identification of B7-H4 receptor expressing cells, for the identification of new cell types, and for studies into the molecular mechanisms of action of B7-H4.

Additionally, bioactive agents may be screened for B7-H4 receptor agonistic or antagonistic activity. Accordingly, methods of screening for additional bioactive agents that function as B7-H4 receptor agonists or antagonists are also provided. In one embodiment, candidate bioactive agents are screened for their ability to activate the B7-H4 receptor. In another embodiment candidate bioactive agents are screened for their ability to function as antagonists of the B7-H4 receptor. The assays preferably utilize human B7-H4 receptors, although other B7-H4 receptors may also be used.

Bioactive agents that may be screened for B7-H4 receptor agonist or antagonist activity include, but are not limited to, proteins, small organic molecules, carbohydrates (including polysaccharides), polynucleotides and lipids. Generally a plurality of assay mixtures are run in parallel with different agent concentrations to obtain a differential response to the various concentrations. Typically, one of these concentrations serves as a negative control, i.e., at zero concentration or below the level of detection. In addition, positive controls, i.e. the use of agents known to agonize or antagonize B7-H4 receptors may be used.

Candidate agents encompass numerous chemical classes, though typically they are organic molecules, preferably small organic compounds having a molecular weight of more than 100 and less than about 2,500 daltons, more preferably between 100 and 2000, more preferably between about 100 and about 1250, more preferably between about 100 and about 1000, more preferably between about 100 and about 750, more preferably between about 200 and about 500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Additional candidate agents include peptidomimetics. Peptidomimetics can be made as described, e.g., in WO 98/156401. Peptidomimetics, as used herein, refers to molecules which mimic peptide structures. Peptidomimetics have general features analogous to their parent structures, polypeptides, such as amphiphilicity. Examples of such peptidomimetic materials are described in Moore et al., Chem. Rev. 101(12), 3893-4012 (2001) and Gentilucci, et al., Curr. Med. Chem., 13(20):2449-66 (2006). Peptidomimetics have been developed in a number of classes, such as peptoids, retro-inverso peptides, azapeptides, urea-peptidomimetics, sulphonamide peptides/peptoids, oligoureas, oligocarbamates, N,N′-linked oligoureas, oligopyrrolinones, oxazolidin-2-ones, azatides, and hydrazino peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs. In a preferred embodiment, the candidate bioactive agents are organic chemical moieties or small molecule chemical compositions, a wide variety of which are available in the art.

In an exemplary assay cells expressing a neuropilin, a plexin, a semaphorin, and/or cells that secrete, or cause other cells to secrete inflammatory cytokines, can be used to identify antibodies, small molecules, and other modulators that are agonists or antagonists of B7-H4 receptors, or B7-H4. For example, in the presence of a B7-H4 fusion protein and a stimulatory signal, an antagonist such as a neutralizing antibody would increase the production of IL-17 or IFN-γ from T cells, or reduce the level of IL-10 production from Tregs. In a specific example, the antagonist is a neutralizing anti-B7-H4 antibody, or a neutralizing anti-B7-H4 receptor antibody. Other outcomes or end points for screening receptor agonist and antagonists will be understood to one of skill in the art based on the functions and activity of the agonists and antagonists described herein.

### VII. Methods of Use

Methods of modulating immune responses by modulating a neuropilin or a plexin signal transduction pathways using the disclosed receptor antagonists and agonists are disclosed. Antagonists typically block or reduce signal transduction through a neuropilin or a plexin, while agonists typically promote or increase signal transduction through a neuropilin or a plexin. Modulation of neuropilin or plexin signal transduction results in modulation of an immune response. Accordingly, methods for promoting or inhibiting immune responses are provided and can include administering an agonist or antagonist of neuropilin or plexin signal transduction to a subject in need thereof.

B7-H4/B7-H4 receptor signal transduction can trigger events in the immune response pathway. Therefore, modulators of B7-H4/B7-H4 receptor signal transduction can be used to mediate, for example Th1 and Th17 and Treg responses, and treat diseases associated therewith as discussed in more detail below.

Administration is not limited to the treatment of existing conditions, diseases or disorders (i.e. an existing cancer or infection) but can also be used to prevent or lower the risk of developing such diseases in an individual, i.e., for prophylactic use. Potential candidates for prophylactic treatment include individuals with a high risk of developing cancer or contracting an infection or infectious disease, and transplant candidates and recipients.

A number of roles for Nrp-1 in immune response regulation have been identified. For example, Nrp-1 has been proposed to play a role in the interaction of Treg cells with DCs. Nrp-1 is preferentially expressed on Treg cells and can be induced by ectopic expression of Foxp3 in Foxp3− T cells (Sarris et al., Immunity, 28:402-413 (2008), and reviewed in Shevach, Immunity, 30:636-645 (2009)). Nrp-1 promotes long interactions between Treg cells and immature DCs. Blocking of Nrp-1 decreases the frequency of long interactions, whereas ectopic expression of Nrp-1 in Foxp3− T cells increases the number of long interactions. Anti-Nrp-1 completely abrogates suppression of proliferation mediated by Treg cells when the responder T cells are stimulated with low concentrations of antigen. These data indicate that the role of Nrp-1 is to give Treg cells a head start over naive responder T cells under conditions in which antigen is limiting.

Accordingly, as discussed in more detail below, in some embodiments, the neuropilin agonists disclosed herein are used to increase Treg activity under low concentrations of antigen. In some embodiments, antagonists of neuropilin are used to reduce suppression of proliferation mediated by Treg cells when the responder T cells are stimulated with low concentrations of antigen.

Solomon, et al., PNAS, 108(5):2040-2045 (2011) reported that overexpression of Nrp-1 attenuates EAE progression and, conversely, the lack of Nrp-1 results in disease aggravation. The increased disease severity occurs in a CD4+ T cell-dependent manner (that skews the balance of helper T cells away from regulatory subtypes toward inflammatory TH-17 subtypes). Solomon, et al., also reported that the suppressive effect of CD4+ T cells from myelin antigen-ECi mice appears to be independent of Foxp3, because the lack of Nrp1 impairs immune suppression without altering Foxp3 expression.

Accordingly, in some embodiments, the neuropilin agonists disclosed herein are used to prevent, reduce, or inhibit one or more symptoms of an inflammatory or autoimmune disease or disorder by restoring Treg function.

Additionally, it has been reported that function between Treg cells and DCs is unbalanced in the context of rheumatoid arthritis, resulting in an increased number of DCs with a higher ability to capture antigens (Xq, et al., Scand J Rheumatol., 41:413-420 (2012), and that nTregs are unable to inhibit the proinflammatory cytokine production of rheumatoid synoviocytes, potentially limiting the scope for conventional Treg therapy (Beavis, et al., 108(40):16717-22 (2012)).

Accordingly, as discussed in more detail below, in some embodiments, the neuropilin agonists disclosed herein are used to increase the activity or number of Tregs; to decrease the activity or number of dendritic cells or T helper cells, or cytotoxic T cells; to increase the ratio of Tregs to dendritic cells or T helper cells, or cytotoxic T cells; or combinations thereof.

Nrp-1 also acts as a co-receptor for the vascular endothelial growth factor (VEGF). Tumors can produce high levels of VEGF, and Hansen, OncoImmunology 2:2, e23039 (2013), reported elevated levels of Nrp-1-expressing Foxp3+ Tregs around found within tumors and that Nrp-1+ Tregs (but not their Nrp-1-deficient counterparts) migrated in response to recombinant VEGF, leading to a conclusion that tumor-derived VEGF attracts Tregs via Nrp-1, which reduces the immune response to the cancer. Additional data showed that T cell-specific ablation of Nrp-1 results in significantly lower numbers of tumor-infiltrating Foxp3+ Tregs, and the abrogation of tumor-produced VEGF produced a similar phenotype, featuring reduced tumor growth, a comparatively stronger activation of tumor-infiltrating CD8+ T cells and decreased amounts of tumor-infiltrating Tregs.

Accordingly, in some embodiments, the neuropilin antagonists disclosed herein are used to prevent, inhibit, reduce, or block Treg proliferation or differentiation, or one or more Treg functions. The methods can be used, for example, to prevent Treg inhibition of an immune response against cancer.

Such antagonists can be used in the treatment of autoimmune disease by inhibiting the interaction of DCs and Nrp-1+ Th cells, and thus allowing the interaction of DCs with Tregs, and the re-establishment of immune tolerance. In a preferred embodiment such antagonists bind preferentially to and/or antagonize Nrp-1 receptor complexes on the surface of Th cells, or the binding of B7-H4 to Th cells.

In one embodiment discussed in more detail below, the Nrp-1 receptor complex antagonist is a B7-H4 fusion protein, whereby the relatively low affinity (compared to an anti-Nrp-1 or anti-plexin antibody) of the B7-H4 fusion protein results in preferential binding to Nrp-1 high expressing Th cells, without compromising the ability of B7-H4 expressing DCs to bind Nrp-1 low expressing Tregs, thus allowing for the re-establishment of immune tolerance. In a preferred embodiment the Nrp-1 receptor complex is a fusion protein including the extracellular domain of B7-H4 fused to the Fc region of an immunoglobulin protein. Selection of the antagonist for Nrp-1 high expressing Th cells can also be addressed through careful dosing of the antagonist.

For example, a method of inducing or re-establishing immune tolerance can include administering to a subject in need thereof an effective amount of a B7-H4-Ig fusion protein to decrease interaction between dendritic cells and Th cells in the subject. The agonist can modulate pro-inflammatory dendritic cell response or Th/Treg cell differentiation/balance in the subject. In some embodiments, the interaction between dendritic cells and Tregs is increased. The dendritic cells can be mature dendritic cells, and the interaction between the dendritic cells and the Th cells can be Nrp-1 dependent. In some embodiments, Th activity is reduced, Treg activity is increased, the ratio of Treg activity to Th cell activity is increased, or a combination thereof. The methods can be used to treat subjects with inflammatory and autoimmune diseases and disorders, for example, by reducing one or more symptoms thereof.

Method of modulating immune response pathways using the disclosed B7-H4 receptors antagonists and agonists are discussed in more detail below.

A. Methods of Using B7-H4 Receptor Antagonists

The disclosed B7-H4 receptor antagonists can be used to increase an immune stimulatory response, to decrease an immune inhibitory response, or a combination thereof. By inhibiting, reducing, or blocking the biological activity of B7-H4 receptors, B7-H4 receptor antagonists can be used to maintain, prolong, or enhance activation of T cells, as inhibiting, reducing, or blocking B7-H4 receptor biological activity can inhibit the suppression or attenuation of T cell activation or functional activity that would otherwise occur.

The antagonists of B7-H4 receptors can be used alone or in combination. Inhibition of B7-H4 receptor activity is typically compared to an appropriate control or predetermined amount of activity using conventional methods. For example, threshold B7-H4 receptor activity in a host can be determined prior to administration of B7-H4 receptor antagonists. B7-H4 receptor activity after administration of that antagonist that is lower than the threshold B7-H4 receptor activity demonstrates an inhibition of B7-H4 receptor activity and stimulation or enhancement of an immune response.

1. Methods of Modulating Immune Responses with B7-H4 Receptor Antagonists

An immune response can be induced, maintained, prolonged or enhanced in a host, preferably a human host, by inhibiting, reducing or blocking the biological activity or expression of B7-H4 receptors in the host. Interfering with the activity of B7-H4 receptors, for example by blocking binding of its natural ligand, will inhibit or reduce the inhibition of mature dendritic cell, Tfh cells, or a T cell response by B7-H4 Inhibiting or reducing the activity of B7-H4 receptors allows for an immune response to initiate and progress and increase the response of the immune system to infections and cancer. Thus, an immune response against cancer or infection can be enhanced, maintained or prolonged by administering a therapeutically effective amount of a B7-H4 antagonist to a host.

Receptor antagonists can also be administered in an effective amount to decrease Treg responses, proliferation, or differentiation; or increase Th1 or Th17 cell responses, proliferation, or differentiation. In some embodiments, the receptor antagonist directly blocks or reduces an inhibitory signal. In some embodiments the antagonist also leads to an indirect or downstream decrease in an immune inhibitory response, or an increase in an immune stimulating response.

Methods for treating diseases associated with elevated levels of B7-H4 and B7-H4 receptors are also provided. Subjects with diseases characterized by elevated levels of B7-H4, for example, certain cancers, can be administered an effective amount of B7-H4 receptor antagonist to reduce B7-H4-mediated immune suppression and enhance an immune response against the disease. For example, B7-H4 receptor antagonists can be used to target B7-H4+ tumors, or other cell types expressing B7-H4 such as tumor associated macrophages and for ADC (protein) targeting as indicated above.

In particular embodiment, B7-H4 is expressed on the surface of tumor cells (and infiltrating DCs) and binds to B7-H4 receptors on, for example, effector T cells, mature DC and B cells, leading to immune suppression. Antagonists of B7-H4 or B7-H4 receptors can overcome this immune suppression by inhibiting the interaction of tumor cells with effector T cell or mature DC and thus break immune suppression, allowing for the activation of Th cells and the enhancement of effector immune functions.

Receptor antagonists can also have direct or indirect anti-tumor activity. For example. Qian, et al (Qian, et al., Cell Tissue Res, 2013 May 10. [Epub ahead of print]) showed that B7-H4 enhances oncogenicity and inhibits apoptosis in cancer cells. Furthermore, disrupting B7-H4 function on tumor cells prevents tumor cell growth through a number of processes, such as increased caspase activity and apoptosis, and inhibition of the Erk 1/2 signaling pathway. Therefore, antagonists that prevent the binding B7-H4 to its receptor as described herein, can also prevent tumor growth through many processes, including the induction of apoptosis, inhibition of tumor cell proliferation and migration, and inhibition of the Erk1/2 pathway.

B7-H4 receptor antagonists can be used in combination for enhanced efficacy. For example, antibodies that bind to B7-H4 receptors and anti-B7-H4 antibodies can be combined such that anti-receptor antibody targets mature DC and blocks immune evasion, whereas anti-B7-H4 targets the tumor cell directly. In a preferred embodiment, the anti-B7-H4 antibody has direct anti-tumor activity such as ADCC, CDC or ADC.

2. Inhibition of Tumor-Associated Macrophage Mediated Immune Suppression

The association between inflammation and cancer dates back more than a century to observations noting infiltration of large numbers of white blood cells into tumor sites (Balkwill, F. et al. (2001) “Inflammation And Cancer: Back To Virchow?,” Lancet 357:539-545; Coussens, L. M. et al. (2002) “Inflammation and Cancer,” Nature 420:860-867). Several studies have now identified two main pathways linking inflammation and cancer: an intrinsic and an extrinsic pathway (Allavena, P. et al. (2008) “Pathways Connecting Inflammation and Cancer,” Curr. Opin. Genet. Devel. 18:3-10; Colotta, F. (2009) “Cancer-Related Inflammation, The Seventh Hallmark of Cancer: Links to Genetic Instability,” Carcinogenesis 30(7): 1073-1081; Porta, C. et al. (2009) “Cellular and Molecular Pathways Linking Inflammation and Cancer,” Immunobiology 214:761-777). The intrinsic pathway includes genetic alterations that lead to inflammation and carcinogenesis, whereas the extrinsic pathway is characterized by microbial/viral infections or autoimmune diseases that trigger chronic inflammation in tissues associated with cancer development. Both pathways activate pivotal transcription factors of inflammatory mediators (e.g., NF-κB, STAT3, and HIF-1) and result in the recruitment of leukocytes that play a key role in inflammation (Solinas, G. et al. (2009) “Tumor-Associated Macrophages (TAM) As Major Players Of The Cancer-Related Inflammation,” J. Leukoc. Biol. 86(5):1065-1073).

Tumor-associated macrophages (TAMs) provide a link between inflammation and cancer. Macrophages are immune system cells derived from activated blood monocytes. They are primarily recognized as participating in inflammatory responses induced by pathogens or tissue damage by acting to remove (i.e., phagocytose) pathogens, dead cells, cellular debris, and various components of the extra-cellular matrix (ECM). Macrophages have been found to constitute an important constituent in the tumor microenvironment and to represent up to 50% of the tumor mass.

In addition to mediating phagocytosis, macrophages secrete pro-angiogenic growth factors and matrix-remodeling proteases, and thus play a role in the development of the vascular infrastructure (i.e., angiogenesis) needed for tumor development and growth (Pollard, J. W. (2009) “Trophic Macrophages In Development And Disease,” Nat. Rev. Immunol. 9:259-270). As such, the presence of macrophages within a tumor appears to assist the growth of the tumor. A number of studies provide evidence that the presence of tumor-associated macrophages within the tumor is a negative prognostic factor of survival (Farinha, P. et al. (2005) “Analysis Of Multiple Biomarkers Shows That Lymphoma-Associated Macrophage (LAM) Content Is An Independent Predictor Of Survival In Follicular Lymphoma (FL),” Blood 106:2169-2174; Dave, S. S. et al. (2004) “Prediction Of Survival In Follicular Lymphoma Based On Molecular Features Of Tumor-Infiltrating Immune Cells,” N. Engl. J. Med. 351:2159-2169; Solinas, G. et al. (2009) “Tumor-Associated Macrophages (TAM) As Major Players Of The Cancer-Related Inflammation,” J. Leukoc. Biol. 86(5):1065-1073).

B7-H4 has been shown to be over-expressed in tumor associated macrophages (TAMs) including those present in ovarian tumors (Kryczek, I. et al. (2006) “B7-H4 Expression Identifies A Novel Suppressive Macrophage Population In Human Ovarian Carcinoma,” J. Exp. Med. 203(4):871-881; Kryczek, I. et al. (2007) “Relationship Between B7-H4, Regulatory T Cells, And Patient Outcome In Human Ovarian Carcinoma,” Cancer Res. 67(18):8900-8905).

Tumors typically need to generate their own vasculature to enable oxygen and nourishment delivery to the expanding tumor cells. Thus, the progression of tumors requires coordinated signaling between tumor cells and non-malignant cells in the tumor microenvironment (Kaler, P. et al. (2010) “Tumor Associated Macrophages Protect Colon Cancer Cells from TRAIL-Induced Apoptosis through IL-1β-Dependent Stabilization of Snail in Tumor Cells,” PLos ONE 5(7):e11700 1-13). It is now well established that tumor-associated macrophages (TAMs), as well as neutrophils, fibroblasts and other cells cooperate with tumor cells to facilitate angiogenesis in tumors (Nucera, S. et al. (2011) “The Interplay Between Macrophages And Angiogenesis In Development, Tissue Injury And Regeneration,” Int. J. Dev. Biol. doi: 10.1387/ijdb.103227sn; Zamarron, B. F. et al. (2011) “Dual Roles Of Immune Cells And Their Factors In Cancer Development And Progression,” Int. J. Biol. Sci. 7(5):651-658; Liu, J. et al. (2011) “Tumor-Associated Macrophages Recruit CCR6+ Regulatory T Cells And Promote The Development Of Colorectal Cancer Via Enhancing CCL20 Production In Mice,” PLoS One. 6(4):e19495; Rigo, A. et al. (2010) “Macrophages May Promote Cancer Growth Via A GM-CSF/HB-EGF Paracrine Loop That Is Enhanced By CXCL12,” Molec. Cancer 9(273):1-13; Lin, J. Y. et al. (2011) “Clinical Significance Of Tumor-Associated Macrophage Infiltration In Supraglottic Laryngeal Carcinoma,” Chin. J. Cancer 30(4):280-286; Vergati, M. (2011) “The Consequence Of Immune Suppressive Cells In The Use Of Therapeutic Cancer Vaccines And Their Importance In Immune Monitoring,” J. Biomed. Biotechnol. 2011:182413).

The high levels of B7-H4 expression found in numerous tumor tissues, for example, human ovarian cancers, points to a key role for B7-H4 in mediating immune suppression. B7-H4+ TAMs have been found to suppress tumor-associated antigen-specific T cell immunity (Kryczek, I. et al. (2006) “B7-H4 Expression Identifies A Novel Suppressive Macrophage Population In Human Ovarian Carcinoma,” J. Exp. Med. 203(4):871-881). The intensity of B7-H4 expression in TAMs correlates significantly with Treg cell numbers in the tumor. Furthermore, B7-H4 expressed on TAMs, is associated with poor patient outcome (Kryczek, I. et al. (2006) “B7-H4 Expression Identifies A Novel Suppressive Macrophage Population In Human Ovarian Carcinoma,” J. Exp. Med. 203(4):871-881). Previously published data also showed that TAMs spontaneously produce chemokine CCL22 that mediates Treg cell trafficking into the tumor, and Treg cell-induced B7-H4 expression on antigen-presenting cells (APC), including TAMs themselves (Kryczek, I. et al. (2006) “Cutting Edge: Induction Of B7-H4 On APCs Through IL-10: Novel Suppressive Mode For Regulatory T Cells,” J. Immunol. 177(1):40-44). Taken together, such findings suggest that B7-H4+ TAMs play a very important role on immune suppression in the tumor microenvironment allowing the tumor to avoid detection by the immune system (“immune evasion”).

Thus, one embodiment provides methods for blocking B7-H4, modulating its surface expression, or depleting B7-H4⁺ tumor associated macrophages using molecules (including anti-B7-H4 antibodies) that are capable of immunospecifically binding to B7-H4 to treat cancer.

Thus, methods for inhibiting tumor-associated macrophage (TAM) mediated immune suppression can include administering to a subject an effective amount of B7-H4 receptor antagonist to reduce or inhibit TAM activity.

3. Diseases to be Treated with Receptor Antagonists

Receptor antagonist can be used to treated diseases including cancer and infectious diseases. The methods typically include administering to a subject in need thereof a receptor antagonist in an effective amount to reduce one or more symptoms of the disease being treated. For example, if the disease is cancer, the treatment can reduce tumor burden or prevent tumor growth or spreading. A method of treating cancer in a subject can include administering to a subject an effective amount of a pharmaceutical composition including a B7-H4 antagonist or B7-H4 receptor antagonist to increase or induce apoptosis of tumor cells, reduce or inhibit tumor cell proliferation, reduce or inhibit tumor cell migration, reduce or inhibit the Erk1/2 pathway in tumor cells, or a combination thereof.

If the disease is infectious, the treatment can be effective to reduce one or more pathologies associated with the infection, or reduce or prevent growth or spreading of the infection or infectious agent causing the infection.

In some embodiments, the subject is one with elevated levels of B7-H4. Accordingly, patients with elevated expression of B7-H4 relative to control can be selected for treatment with antagonists of B7-H4 receptors.

a. Cancer

Method of treating cancer and of increasing an immune response to cancer in a subject with cancer using B7-H4 receptor antagonists are disclosed. In some embodiments, a method for enhancing, maintaining or prolonging an immune response in host for treating cancer includes administering an amount of a B7-H4 receptor polypeptide antagonist effective to inhibit or reduce inhibition of T cells by B7-H4 expressed on the tumor or TAM in the host that provides an immunosuppressive signal. In such cases, B7-H4 antagonists can break immune suppressive and trigger anti-tumor immune responses.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

Cancers that can be treated include, but are not limited to, bladder, brain, breast, cervical, colo-rectal, esophageal, kidney, liver, lung, nasopharangeal, pancreatic, prostate, skin, stomach, uterine, ovarian, and testicular cancers.

In some embodiments the receptor antagonists described herein contain domains that bind to antigens, ligands or receptors that are specific to tumor cells or tumor-associated neovasculature, or are upregulated in tumor cells or tumor-associated neovasculature compared to normal tissue.

In one embodiment the antagonist, for example a receptor fusion protein, contains a domain that specifically binds to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

A tumor antigen may include a cell surface molecule. Tumor antigens of known structure and having a known or described function, include the following cell surface receptors: HER1 (GenBank Accession No. U48722), HER2 (GenBank Acc. Nos. X03363 and M17730), HER3 (GenBank Acc. Nos. U29339 and M34309), HER4 (GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043), insulin-like growth factor-II (GenBank Acc. Nos. X03562, X00910, M17863 and M17862), transferrin receptor (GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (GenBank Acc. Nos. M65132 and M64928) NY-ESO-1 (GenBank Acc. Nos. AJ003149 and U87459), Melan-A/MART-1 (GenBank Acc. Nos. U06654 and U06452), tyrosinase (GenBank Acc. No. M26729), Gp-100 (GenBank Acc. No. S73003), MAGE (GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Protein therapeutics can be ineffective in treating tumors because they are inefficient at tumor penetration. Tumor-associated neovasculature provides a readily accessible route through which protein therapeutics can access the tumor. In another embodiment the antagonist contains a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed antagonists.

b. Infections

Method of treating infections and of increasing an immune response to infectious agent in a subject with an infection using B7-H4 receptor antagonists are disclosed. In some embodiments, enhancing, maintaining or prolonging an immune response in a host is desirable, for example, when the host suffers from a viral infection, bacterial infection, fungal, protozoa infection. Thus, one embodiment provides a method for treating an infection by administering an amount of a B7-H4 receptor antagonist effective to inhibit or reduce down regulation of T cells by B7-H4 in the host.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni.

B. Methods of Using B7-H4 Receptor Agonists

The disclosed B7-H4 receptor agonists can be used to decrease an immune stimulatory response, decrease an inflammatory response, to increase an immune inhibitory response, to decrease autoimmune disease, to restore immune tolerance, or a combination thereof. By increasing signal transduction through B7-H4 receptors, B7-H4 receptor agonists can be used to inhibit, reduce, or block activation of T cells, and leading to the suppression or attenuation of T cell activation or functional activity that would otherwise occur.

The agonists of B7-H4 receptors can be used alone or in combination. Activation of B7-H4 receptor activity is typically compared to an appropriate control or predetermined amount of activity using conventional methods. For example, threshold B7-H4 receptor activity in a host can be determined prior to administration of the B7-H4 receptor agonist. B7-H4 receptor activity after administration of that agonist that is higher than the threshold B7-H4 receptor activity demonstrates an activation of B7-H4 receptor activity and suppression or reduction of an immune response.

1. Methods of Modulating Immune Responses with B7-H4 Receptor Agonists

Receptor agonists can be administered in an effective amount to decrease Treg responses, proliferation, or differentiation; increase Th1 or Th17 cell responses, proliferation, or differentiation include administering an effective amount of an agonist of B7-H4 signal transduction to enhance signal transduction through a B7-H4 receptor. In some embodiments, the receptor agonist directly induces an inhibitory signal, for example by suppressing effector T cell or Tfh cell response.

In some embodiments, the receptor agonists disclosed herein are used to reduce effector T cell or Tfh cell proliferation or differentiation. The methods can be used, for example, to reduce overactive T cell responses.

The agonists can be used to establish or re-establish immune tolerance in a subject in need thereof. For example, a method of inducing or re-establishing immune tolerance can include administering to a subject in need thereof an effective amount of a B7-H4 receptor agonist to modulate dendritic cell pro-inflammatory response. Receptor agonists may affect DC cytokine profile, for example, IL-6 and IL-10, which can modulate Treg and Th17 differentiation/balance in the host. Such agonists can be used in the treatment of autoimmune disease by selectively inhibiting the interaction of DCs and receptor positive Th cells, and thus allowing the interaction of DCs with Tregs, and the re-establishment of immune tolerance. In some embodiments, the interaction between dendritic cells and Tregs is increased. The dendritic cells can be mature dendritic cells, and the interaction between the dendritic cells and the Th cells can be dependent on a neuropilin alone or in combination with a plexin. In some embodiments, Th activity is reduced, Treg activity is increased, the ratio of Treg activity to Th cell activity is increased, or a combination thereof.

In particular embodiments the Nrp-1 receptor complex agonist is a B7-H4 fusion protein, whereby the relatively low affinity (compared to an anti-Nrp-1 or anti-plexin antibody) of the B7-H4 fusion protein results in preferential binding to Nrp-1 high expressing Th cells, without compromising the ability of B7-H4 expressing DCs to bind Nrp-1 low expressing Tregs, thus allowing for the re-establishment of immune tolerance. In a preferred embodiment the Nrp-1 receptor complex is a fusion protein comprising the extracellular domain of B7-H4 fused to the Fc region of an immunoglobulin protein. Selection of the antagonist for Nrp-1 high expressing Th cells can also be addressed through careful dosing of the antagonist.

Agonists can also be used in the treatment of autoimmune disease by suppressing the function of mature DC, Tfh cells and B cells and establishing immune tolerance. In a preferred embodiment such agonists bind preferentially to and/or agonize B7-H4 receptors on the surface of DC, Tfh or B cells.

Administration is not limited to the treatment of existing conditions, diseases or disorders (i.e. an existing inflammatory or autoimmune disease or disorder) but can also be used to prevent or lower the risk of developing such diseases in an individual, i.e., for prophylactic use. Potential candidates for prophylactic vaccination include individuals with a high risk of developing an inflammatory or autoimmune disease or disorder, i.e., with a personal or familial history of certain types of autoimmune disorders and transplant rejection.

For example, a method of inducing or re-establishing immune tolerance can include administering to a subject in need thereof an effective amount of a B7-H4-Ig fusion protein to decrease interaction between dendritic cells and Th cells in the subject. In some embodiments, the interaction between dendritic cells and Tregs is increased. The dendritic cells can be mature dendritic cells, and the interaction between the dendritic cells and the Th cells can be Nrp-1 dependent. In some embodiments, Th activity is reduced, Treg activity is increased, the ratio of Treg activity to Th cell activity is increased, or a combination thereof.

The method can be employed to treat subjects with an inflammatory or autoimmune disease/disorder, for example, by reducing one or more symptoms of the inflammatory or autoimmune disease/disorder. In some embodiments, the Th cells in the subject overexpress Nrp-1 compared to Th cells in a control subject, the Th cells have greater cell surface expression of Nrp-1 than the Treg cells, or a combination thereof. In some embodiments, the B7-H4 fusion protein has lower affinity for an anti-B7-H4 receptor complex than anti-Nrp-1 antibody or anti-plexin antibody resulting in preferential binding to Nrp-1 high expressing Th cells, without compromising the ability of B7-H4 expressing dendritic cells to bind Nrp-1 low expressing Tregs. Preferred B7-H4-Ig fusions protein include the amino acid sequence of SEQ ID NO:22, 23, 24, or 25.

2. Diseases to be Treated with Receptor Agonists

a. Inflammation and Autoimmunity

Chronic and persistent inflammation is a major cause of the pathogenesis and progression of systemic autoimmune diseases such as rheumatoid arthritis (RA) and systemic lupus erythematosus (SLE). B7-H4 inhibits cell cycle progression of T cells in the presence of antigen stimulation through activation of B7-H4 receptors expressed on the T cell surface. B7-H4 can also inhibit innate immunity by suppressing proliferation of neutrophil progenitors.

Additionally, it has been reported that function between Treg cells and DCs is unbalanced in the context of rheumatoid arthritis, resulting in an increased number of DCs with a higher ability to capture antigens (Xq, et al., Scand J. Rheumatol., 41:413-420 (2012), and that nTregs are unable to inhibit the proinflammatory cytokine production of rheumatoid synoviocytes, potentially limiting the scope for conventional Treg therapy (Beavis, et al., 108(40):16717-22 (2012)).

Accordingly, in some embodiments, the B7-H4 receptor agonists disclosed herein are used to decrease the activity or number of mature dendritic cells or T helper cells, or cytotoxic T cells; to increase the ratio of Tregs to dendritic cells or T helper cells, or cytotoxic T cells; to treat diseases associated with elevated levels of a neuropilin or a plexin; or to treat subjects with elevated expression of a neuropilin or a plexin.

The methods can be employed to treat subjects with an inflammatory or autoimmune disease/disorder, for example, by reducing, preventing, or inhibiting one or more symptoms of the inflammatory or autoimmune disease/disorder. An immune response can be inhibited or reduced in a host, preferably a human host, by stimulating the biological activity of B7-H4 receptors in the host. Therefore, an inflammatory response can be reduced or inhibited by agonizing the biological activity of B7-H4 receptors in vivo, for example, by administering to an individual in need thereof an effective amount of a B7-H4 receptor agonist to inhibit or decrease one or more symptoms of the disease.

Compositions and methods for treating autoimmune and inflammatory diseases/disorders by using a B7-H4-Ig fusion protein to increase B7-H4 mediated signaling are discussed in, for example, U.S. Published Application Nos. 2012/0177645 and 2012/0276095; and compositions and methods for interfering with the biological activity of soluble B7-H4 are discussed in, for example, U.S. Pat. Nos. 7,931,896 and 7,989,173, and U.S. Published Application No. 2009/0142342, each of which is specifically incorporated by reference herein in its entirety.

Representative inflammatory or autoimmune diseases and disorders that may be treated using B7-H4 receptor agonists include, but are not limited to, transplant rejection, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

Inflammatory or autoimmune diseases and disorders including transplant rejection can be inhibited or reduced in a subject by administering an effective amount of a B7-H4 receptor agonist to inhibit or reduce the biological activity of an immune cell or to reduce the amounts of proinflammatory cytokines. Exemplary proinflammatory cytokines include, but are not limited to IL-1β, TNF-α, TGF-beta, IFN-γ, IL-17, IL-6, IL-23, IL-22, IL-21, and MMPs.

A B7-H4 receptor agonist can also be used to target other cells that secrete, or cause other cells to secrete, inflammatory cytokines. In a preferred embodiment, a B7-H4 receptor agonist is used to target neutrophils. It is believed that B7-H4 receptor agonists reduce neutrophil proliferation, decreases secretion of inflammatory cytokines, and/or reduce recruitment of additional neutrophils and/or other inflammatory cells.

For transplant rejection therapy, the transplanted material to be treated with a B7-H4 receptor agonist can be cells, tissues, organs, limbs, digits or a portion of the body, preferably the human body. The transplants are typically allogenic or xenogeneic. B7-H4 receptor agonist can be administered systemically or locally. In some embodiments, B7-H4 receptor agonist is administered to a site of transplantation prior to, at the time of, or following transplantation. In one embodiment, B7-H4 receptor agonist are administered to a site of transplantation parenterally, such as by subcutaneous injection. In other embodiments, or B7-H4 receptor agonist are administered ex vivo directly to cells, tissue or organ to be transplanted. In one embodiment, the transplant material is contacted with B7-H4 receptor agonist prior to transplantation, after transplantation, or both. In other embodiments, B7-H4 receptor agonist is administered to immune tissues or organs, such as lymph nodes or the spleen.

A B7-H4 receptor agonist can be administered in combination with one or more additional therapeutic agents, including, but not limited to, antibodies against other lymphocyte surface markers (e.g., CD40) or against cytokines, other fusion proteins, e.g., CTLA4-Ig (Orencia®), TNFR-Ig (Enbrel®), anti-TNF (Humira) or other immunosuppressive drugs, anti-proliferatives, cytotoxic agents, or other compounds that may assist in immunosuppression. In one embodiment, the additional therapeutic agent is a CTLA-4 fusion protein, such as CTLA-4 Ig (abatacept). In a preferred embodiment, the additional therapeutic agent is a CTLA4-Ig fusion protein known as belatacept that contains two amino acid substitutions (L104E and A29Y) that markedly increase its avidity to CD86 in vivo.

Still another embodiment provides methods and compositions for treating one or more symptoms of graft versus host disease (GVHD) in a subject in need thereof by administering an effective amount of B7-H4 receptor agonist to alleviate one or more symptoms associated with GVHD.

b. Angiogenesis

Uncontrolled or unwanted angiogenesis can be inhibited or reduced in a host, preferably a human host, by stimulating the biological activity of B7-H4 receptors in the host. Angiogenesis is a critical event in a number of diseases and disorders including rheumatoid arthritis. The process of neovascularization depends on cross-talk between the immune system and the vasculature. For example VEGF stimulates mitogenesis and cell migration in endothelial cells, but may also influence the biological activity of T cells by serving as a chemoattractant and/or enhancing antigen-induced cytokine production in T cells such as Th1, Th2, and Th17 cells (Basu, et al., J. Immunology, 184:545-549 (2010), epub (Dec. 11, 2009)). Additionally, neutrophils are thought to contribute to angiogenic switching by affecting the release of VEGF. Furthermore, secretion of IL-17, such as by Th-17 cells, increases endothelial cell migration, tube formation, and blood vessel development in rheumatoid arthritis (Pickens, et al., J. Immunology, epub. Feb. 19, 2010). Therefore, angiogenesis can be reduced or inhibited by agonizing the biological activity of B7-H4 receptors in vivo, for example, by administering to an subject, preferably in need thereof an effective amount of a B7-H4 receptor agonist to limit, terminate or attenuate T cell responses, and/or limit, terminate, or attenuate mitogenic activity or proliferation of neurophils or endothelial cells.

c. Reducing Soluble B7-H4 Levels

Soluble forms of B7-CD28 family molecules are implicated in the progression of rheumatoid diseases. A recent study shows that soluble B7-H4 could be detected in rheumatoid arthritis (RA) patients and the levels of soluble B7-H4 are correlated with TNF-alpha concentration in synovial fluid.

Soluble B7-H4 (sH4) has also been detected in ovarian cancer patients as a potential biomarker, but the mechanism of production of sH4 is unknown. It has also been discovered that sH4 is found in sera of approximately two-thirds of RA and one third of systemic lupus erythematosus (SLE) patients sampled, and the concentration of sH4 correlates closely with the severity of RA. In an experimental model of RA and SLE, the effect of sH4 was recapitulated, and it was demonstrated that sH4 acts as a decoy to block suppressive functions of endogenous B7-H4, leading to exacerbation of systemic autoimmune diseases. The results demonstrate a role of sH4 in the pathogenesis of systemic autoimmune diseases.

It is believed that elevated levels of sH4 block the inhibitory effect of endogenous B7-H4 resulting in inflammatory and autoimmune diseases and disorders. Therefore, an inflammatory response or an autoimmune disorder can be treated by interfering with the biological activity of sH4.

In one embodiment, sH4 is removed from an individual's blood or plasma ex vivo by contacting it with B7-H4 receptor polypeptides. Blood or plasma is removed from an individual and sH4 is selectively removed from the blood or plasma by contacting the blood or plasma with B7-H4 receptor polypeptides or fragments thereof. The B7-H4 receptor polypeptides or fragments thereof can be immobilized on a substrate. The treated blood or plasma is then returned to the individual.

In another embodiment, soluble B7-H4 receptors or fragments thereof are administered to an individual to reduce the bioavailability of sH4 in vivo.

### VIII. Combination Therapies

Combination therapies are also disclosed. For example, in some embodiments, two or more B7-H4 receptor agonists are administered to a subject. In some embodiments, two or more B7-H4 receptor antagonists are administered to a subject. The compositions can include, or be administered in combination with a co-stimulatory molecule. For example, receptor antagonists can be administered in combination with a co-stimulatory molecule that co-stimulates an immune stimulatory response, for example 4-1BB, CD27, OX40 and CD28H, and/or checkpoint blockers including, but not limited to, anti-PD-1 and anti-PD-Ll. Receptor agonists can be administered in combination with a co-inhibitory molecule that induces an immune inhibitory response, for example CTLA4Ig.

The disclosed compositions can include, or can be administered to a subject in need thereof alone or in combination with one or more additional therapeutic agents. The additional therapeutic agents are selected based on the condition, disorder or disease to be treated. For example, B7-H4 receptor agonists can be co-administered with one or more additional agents that function to inhibit, reduce or suppress the activation of immune responses. Alternatively, B7-H4 receptor antagonists useful to activate or sustain immune responses can be co-administered with one or more chemotherapeutic or anti-viral agents. In a preferred embodiment the additional therapeutic agent targets a different pathway so that the combined effect of the therapies is greater than each alone.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second). The additional therapeutic agents can be administered locally or systemically to the subject, or coated or incorporated onto, or into a device or graft.

A. Immunosuppressive Agents

Suitable immunosuppressive agents include, but are not limited to, antibodies against other lymphocyte surface markers (e.g., CD40) or against cytokines, fusion proteins, e.g., CTLA4-Ig, or other immunosuppressive drugs (e.g., cyclosporin A, FK506-like compounds, rapamycin compounds, or steroids), anti-proliferatives, cytotoxic agents, disease-modifying antirheumatic drugs (DMARDs), or other compounds that may assist in immunosuppression.

As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary Rapamycin compounds are known in the art (See, e.g. Abraham and Gibbons, Clin. Cancer Res., 13(11):3109-14 (2007), WO 95122972, WO 95116691, WO 95104738, U.S. Pat. Nos. 6,015,809; 5,989,591; U.S. Pat. Nos. 5,567,709; 5,559,112; 5,530,006; 5,484,790; 5,385,908; 5,202,332; 5,162,333; 5,780,462; 5,120,727).

The language “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds include, for example, those described in WO 00101385. Preferably, the language “rapamycin compound” as used herein does not include FK506-like compounds.

In a particular combination, the disclosed compositions are administered in combination with one or more disease-modifying antirheumatic drugs (DMARDs). DMARDs typically work by modulating the underlying processes that cause certain forms of inflammatory arthritis including rheumatoid arthritis (RA), ankylosing spondylitis, and psoriatic arthritis. These drugs not only treat arthritis symptoms, but they also can slow down progressive joint destruction, and are thereof useful in treating a variety of inflammatory and autoimmune diseases/disorder such as those disclosed herein. Some of these medications can also be used to treat other conditions, such as cancer or inflammatory bowel disease, or to reduce the risk of rejection of a transplanted organ. Exemplary DMARDs include, but are not limited to, hydroxychloroquine (PLAQUENIL®), leflunomide (ARAVA®), cyclosporine (NEORAL®), sulfasalzine (AZULFIDINE®), methotrexate (RHEUMATREX®, TREXALL®), azathioprine (IMURAN®), cyclophosphamide (CYTOXAN®), and various other biologics such as tocilizumb (ACTEMRA®), certolizumab pegol (CIMZIA®), etanercept (ENBREL®), adalimumab (HUMIRA®), anakinra (KINERET®), abatacept (ORENCIA®), infliximab (REMICADE®), rituximab (RITUXAN®), and golimumab (SIMPONI®).

Other suitable therapeutics include, but are not limited to, anti-inflammatory agents. The anti-inflammatory agent can be non-steroidal, steroidal, or a combination thereof.

B. Anti-Cancer Therapies

The stimulation of an immune response against a cancer may be coupled with surgical, chemotherapeutic, radiologic, hormonal and other immunologic approaches in order to affect treatment.

For example, B7-H4 receptor antagonists can be administered with an antibody or antigen binding fragment thereof specific for a growth factor receptors or tumor specific antigens. Representative growth factors receptors include, but are not limited to, epidermal growth factor receptor (EGFR; HER1); c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II R/M-6-P receptor); insulin receptor related kinase (IRRK); platelet-derived growth factor receptor (PDGFR); colony-stimulating factor-1receptor (CSF-1R) (c-Fms); steel receptor (c-Kit); Flk2/Flt3; fibroblast growth factor receptor 1 (Flg/Cek1); fibroblast growth factor receptor 2 (Bek/Cek3/K-Sam); Fibroblast growth factor receptor 3; Fibroblast growth factor receptor 4; nerve growth factor receptor (NGFR) (TrkA); BDNF receptor (TrkB); NT-3-receptor (TrkC); vascular endothelial growth factor receptor 1 (Flt1); vascular endothelial growth factor receptor 2/Flk1/KDR; hepatocyte growth factor receptor (HGF-R/Met); Eph; Eck; Eek; Cek4/Mek4/HEK; Cek5; Elk/Cek6; Cek7; Sek/Cek8; Cek9; Cek10; HEK11; 9 Ror1; Ror2; Ret; Ax1; RYK; DDR; and Tie.

Additional therapeutic agents include conventional cancer therapeutics such as chemotherapeutic agents, cytokines, chemokines, and radiation therapy. The majority of chemotherapeutic drugs can be divided into: alkylating agents, antimetabolites, anthracyclines, plant alkaloids, topoisomerase inhibitors, and other antitumour agents. All of these drugs affect cell division or DNA synthesis and function in some way. Additional therapeutics include monoclonal antibodies and the tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof.

### IX. B7-H4 Receptors and B7-H4 as Biomarkers

Soluble neuropilins, soluble plexins, or soluble B7-H4, or neuropilin autoantibody, plexin autoantibody, semphorin autoantibody, B7-H4 autoantibody or combination thereof can also be used as biomarkers for immune diseases or disorders, including for example autoimmunity. Elevated levels, or an increase over time, of soluble neuropilins, soluble plexins or soluble B7-H4 or membrane bound B7-H4 or neuropilin autoantibody, plexin autoantibody, semaphorin autoantibody, or B7-H4 autoantibody are indicative of an elevated or unregulated immune response. B7-H4 fusion proteins or other agonists of the B7-H4 signal transduction pathway as described herein can be administered to a subject having elevated levels of circulating soluble neuropilins, soluble plexins or soluble B7-H4, or neuropilin autoantibody, plexin autoantibody, or semaphorin autoantibody or B7-H4 autoantibody or combination thereof in an amount effective to reduce or inhibit an immune response in the subject. Neuropilin and/or plexin fusion proteins can also be administered to neutralize high levels of soluble B7-H4. The effectiveness of the treatment can be evaluated by monitoring the levels of soluble neuropilins, soluble plexins, or soluble B7-H4, or neuropilin autoantibody, plexin autoantibody, or semaphorin autoantibody or B7-H4 autoantibody or combination thereof in the subject over time. If the treatment is effective, the relative levels of soluble neuropilin or soluble plexin, or soluble B7-H4, or neuropilin autoantibody, or plexin autoantibody, or semaphorin autoantibody or B7-H4 autoantibody or combination thereof in the subject should decline over time.

### XIII. Detection, Diagnosis, Pharmacodynamics, and Patient Selection

A. Method of Detection

Methods of detecting one or more B7-H4 receptors or co-ligands in a biological sample are also provided. The detection of B7-H4 receptor or co-ligand proteins in a biological sample obtained from a subject is made possible by a number of conventional methods including but not limited to electrophoresis, chromatography, mass spectroscopy and immunoassays. A preferred method includes immunoassays whereby B7-H4 receptor and/or co-ligand proteins are detected by their interaction with a B7-H4 receptor or co-ligand specific antibody. B7-H4 receptor or co-ligand specific antibodies can be used to detect the presence of B7-H4 receptor or co-ligand proteins in either a qualitative or quantitative manner. Exemplary immunoassays that can be used for the detection of B7-H4 receptors and co-ligands include, but are not limited to, radioimmunoassays, ELISAs, immunoprecipitation assays, Western blot, fluorescent immunoassays, and immunohistochemistry.

A biological sample that may contain B7-H4 receptor or co-ligand proteins thereof can be obtained from an individual. If the biological sample is of tissue or cellular origin, the sample is solubilized in a lysis buffer optionally containing a chaotropic agent, detergent, reducing agent, buffer, and salts. The sample can be a biological fluid sample taken from a subject. Examples of biological samples include urine, barbotage, blood, serum, plasma, tears, saliva, cerebrospinal fluid, tissue, lymph, synovial fluid, or sputum etc. In a preferred embodiment, the biological fluid is whole blood, or more preferably serum or plasma. Serum is the component of whole blood that is neither a blood cell (serum does not contain white or red blood cells) nor a clotting factor. It is the blood plasma with the fibrinogens removed. Accordingly, serum includes all proteins not used in blood clotting (coagulation) and all the electrolytes, antibodies, antigens, hormones, and any exogenous substances (e.g., drugs and microorganisms). The sample can be diluted with a suitable diluent before contacting the sample to the antibody.

Generally, a sample obtained from a subject can be contacted with the antibody that specifically binds a B7-H4 receptor or co-ligand. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or ProteinChip® array and can be analyzed by gas phase ion spectrometry as described above.

Immunoassays for the detection of B7-H4 receptor or co-ligand proteins include the ability to contact a biological sample with an antibody specific to a B7-H4 receptor or co-ligand protein under conditions such that an immunospecific antigen-antibody interaction may occur, followed by the detection or measurement of this interaction. The binding of the antibody to B7-H4 receptor or co-ligand proteins may be used to detect the presence and altered production of B7-H4 receptor or co-ligand proteins.

A particularly preferred immunoassay is ELISA. ELISA typically includes the use of two different B7-H4 receptor or co-ligand-specific antibodies: a capture antibody and a detection antibody. In some embodiments an antibody or antigen binding fragment thereof that recognizes a B7-H4 receptor or co-ligand is used to capture most or all of the B7-H4 receptor or co-ligand in the sample. A detection antibody that can recognize most or all of the B7-H4 receptor or co-ligand can be used to determine the total level of B7-H4 receptor or co-ligand in the biological sample. In some embodiments, the detection antibody recognizes a different domain or epitope than the capture antibody.

As discussed above, it is believed that B7-H4 can bind to neuropilin, as well as plexin4A, and a semphorin such as Sema3A or Sema6C. Therefore, in some embodiments, the capture antibody is specific for one protein and the capture antibody is specific for the same protein. In other embodiments, the capture antibody is specific for one protein and the detection antibody is specific for a different protein. For example, in a particular, non-limiting embodiment, the capture agent is specific for neuropilin-1 and the detection antibody is specific for plexin4A or a semaphorin. In some embodiments, a first detection antibody is specific for the same protein as the capture antibody, and a second assay is carried out in which the detection antibody is to a different protein. In this way, the composition of a receptor complex can be determined and characterized.

In some embodiments, the capture antibody recognizes a receptor and the detection antibody recognizes a ligand bound to the receptor, for example a soluble or cell-free B7-H4, a B7-H4 fusion protein, or a semaphorin. Therefore in some embodiments, the detection antibody binds to B7-H4, particularly the extracellular domain of B7-H4, or the second polypeptide of the fusion protein. Therefore in some embodiments, the detection antibody binds to B7-H4, particularly the extracellular domain of B7-H4, or the second polypeptide of the fusion protein. For example, if the second polypeptide of the fusion protein is the Fc region for human IgG1, the antibody can be an anti-human IgG1 Fc antibody. In this way, receptor occupancy of therapeutic B7-H4 fusion protein or cell-free, soluble B7-H4 can be determined In some embodiments, a fusion protein can be distinguished from cell-free B7-H4 only, transmembrane B7-H4 only, or a combination thereof.

Alternatively, in some embodiments, the capture antibody recognizes a ligand and the detection antibody recognizes the receptor to which the ligand is bound.

In some embodiments, anti-B7-H4 receptor antibodies, and methods of detecting B7-H4 receptor proteins can be used to detect the presence and altered production of B7-H4.

B. Diagnosis

A disease or disorder in an individual can be detected by quantifying the amount of one or more B7-H4 receptors or co-ligands thereof in a biological sample of the individual, wherein an elevated or reduced amount of B7-H4 receptor or co-ligand in the individual's biological sample compared to a control (single or more preferably pooled or averaged values of normal individuals in same assay) is indicative of a disorder or disease state. In one embodiment the under expression of B7-H4 receptor protein is indicative of a immunological disorder, for example an autoimmune disorder or inflammation.

In still another embodiment, the level of the B7-H4 receptor or co-ligand is over expressed. Representative B7-H4 receptors that are overexpressed include soluble, cell-free, or otherwise circulating form of the receptor or co-ligand. In some embodiments the ratio of one receptor (e.g., a neuropilin) to another (e.g., a plexin), or to a co-ligand (e.g., a semaphorin) altered compared to a control.

A biological sample includes tissue or biological fluid such as a fluid from the individual, for example, blood, plasma, saliva, lymph, cerebrospinal fluid, or sputum. A control refers to a biological sample from an individual not experiencing the disease or disorder being tested for. In some embodiment, the receptor or co-ligand is a membrane bound, transmembrane or membrane associated receptor or co-ligand. Therefore, in some embodiment, the biological sample is cells obtained from the subject, for example, immune cells.

The amount of B7-H4 receptor or co-ligand in a sample can be determined using conventional techniques such as chromatography, electrophoresis, immunohistochemistry, immunofluorescence, enzyme-linked immunosorbent assays, mass spectrometry, spectrophotometry, or a combination thereof.

The severity of a disorder or a disease can be detected or assessed by quantifying the level of B7-H4 receptor or co-ligand in an individual's biological sample and correlating the amount of B7-H4 receptor in the individual's biological sample with amount B7-H4 receptor or co-ligand expression indicative of different stages of a disorder or disease. The amounts of B7-H4 receptor or co-ligand that correlate to different stages of disease or disorder or different levels of severity can be predetermined by quantifying B7-H4 receptor or co-ligand levels in patients at different stages of, or with different severity of, a disease or disorder. For example, with RA the following classification for severity is typically employed: Class I: No restriction of ability to perform normal activities; Class II: Moderate restriction, but with an ability to perform most activities of daily living; Class III: Marked restriction, with an inability to perform most activities of daily living and occupation; and Class IV: Incapacitation with confinement to bed or wheelchair.

C. Pharmacodynamic Markers

The effectiveness of treatments using receptor agonists and antagonists can be determined by assaying a sample obtained from a subject receiving treatment with the receptor agonist or antagonist for changes in levels of biomarkers such as serum proteins, preferably pro-inflammatory cytokines, chemokines, acute phase markers, and/or antibodies, such as total IgG, or specific disease-related IgG, or other serum proteins for example sH4, as well as the expression levels of B7-H4 receptors and/or ligands. For example, baseline levels of biomarkers in a biological sample obtained from a subject can be determined prior to treatment with a receptor agonist or antagonist. After or during treatment with the receptor agonist or antagonist, biomarker levels in biological samples from the subject can be monitored. A change in biomarker level, for example a decline in proinflammatory cytokine levels, relative to baseline levels indicates that the treatment is effective in reducing one or more symptoms of an inflammatory disorder. Alternatively, the cytokine levels in blood samples from a subject undergoing treatment with a receptor agonist or antagonist can be compared to predetermined levels of biomarkers determined from subjects without the disease or condition being treated. In some embodiments the level of only one biomarker is monitored. In other embodiments, the levels of 2, 3, 4, 5 or more biomarkers are monitored.

The effectiveness of treatments using a receptor agonist or antagonist can also be determined by assaying a sample obtained from a subject receiving treatment with the receptor agonist or antagonist for changes in levels lymphocyte populations, such as increased or decreased numbers of Treg, or decreased or increased numbers of activated Th1 or Th17 cells compared to a control.

In some embodiments, the effectiveness of treatments using a receptor agonist or antagonist is determined by monitoring disease specific markers or symptoms, using methods known in the art. For example imaging can be employed to assess effectiveness of treatment for Multiple Sclerosis, or delayed-type hypersensitive (DTH) can be monitored to assess effectiveness of treatment for lupus.

The effectiveness of treatments using a receptor agonist or antagonist can also be determined by assaying a sample obtained from a subject receiving treatment with the receptor agonist or antagonist for changes in the expression levels of genes, including, but not limited to, those encoding serum proteins, preferably pro-inflammatory cytokines and/or chemokines, as well as secreted factors, cell surface receptors, and transcription factors that are characteristic of Th1, Th17, and Treg cells. Methods of measuring gene expression are well known in the art and include, but are not limited to, quantitative RT-PCR and microarray analysis.

Exemplary markers that can be monitored to determine the effectiveness of treatment with a receptor agonist or antagonist include, but are not limited to, one or more of IL-1β, TNF-α, TGF-beta, IFN-γ, IL-10, IL-17, IL-6, IL-23, IL-22, IL-21, and MMPs. Biomarkers particularly useful for monitoring arthritis include, for example, CRP, ET-1, IL-6, MCP-1, MCP-3, MIP-2 and TNF-α. Another marker useful for monitoring the effectiveness of treatment with a receptor agonist or antagonist is the level of CD73 in a tissue fluid of a patient, see for example WO 09/05352.

Other preferred markers that can be monitored to determine the effectiveness of treatment with a receptor agonist or antagonist include, but are not limited to B7-H4 receptors, such as a neuropilin (e.g., Nrp-1, Nrp-2, etc.), or plexin (e.g., plexin4A); ligands such as semaphorin (Sema3A, Sema6C, etc.), or B7-H4, or combinations thereof.

D. Patient Selection

Methods of determining the level of B7-H4 receptor or co-ligand may also allow the selection of patients most likely to respond to B7-H4 mediated therapy. For example, it is believed that B7-H4 receptor such as a neuropilin alone, or in a complex, mediates an immune inhibitory signal. Therefore, it is believed that inflammatory or autoimmune diseases/disorders are likely associated with lower receptor levels. Such subjects could be selected for treatment with B7-H4 receptor agonist, such as a B7-H4-Ig fusion protein to increase the inhibitory signal and reduce the autoimmune or inflammatory response.

Similarly, some patients with inflammatory or autoimmune diseases/disorders may have low levels of semaphorin (and/or other receptor proteins). Such subjects could be selected for treatment with B7-H4 receptor agonist, such as a recombinant semaphorin or fusion protein thereof alone or in combination with a B7-H4-Ig, to increase the inhibitory signal and reduce the autoimmune or inflammatory response.

In other embodiments, receptor occupancy by cell-free, soluble B7-H4 can be an indicator of an inflammatory or autoimmune diseases/disorder. Receptor occupancy can be determined as discussed above. Subjects with cell-free B7-H4 occupance that is higher than a control can be selected for B7-H4 receptor agonist therapy, for example B7-H4-Ig alone or in combination with a semaphorin or fusion protein thereof.

In some embodiments, patients with increased B7-H4 receptor or co-ligand levels may be more likely to respond to treatment that specifically targets the B7-H4 receptor, and can include both agonists and antagonists of B7-H4. In a preferred embodiment the B7-H4 receptor agonist is a B7-H4 Ig fusion protein.

The effectiveness of the receptor agonists and antagonists disclosed herein can be predicted by pre-screening target patients for levels of biomarkers, or gene expression as described above, or polymorphisms within the genes encoding downstream effector genes.

In a non-limiting example, patients that have elevated levels of one or more inflammatory cytokines or chemokines, relative to a subject that does not have an inflammatory disorder can be selected for treatment with a receptor agonist. Alternatively, patients that have a polymorphism in or more inflammatory cytokine or chemokine genes can be selected for treatment with a receptor agonist. For example, patients with particular polymorphisms within the IL-10 gene may be expected to respond more or less well to treatment with the disclosed compositions, depending on the nature of the polymorphism. Exemplary molecules and their respective genes that can be screened to determine if the treatment will be effective include, but are not limited to, one or more of IL-1β, TNF-α, TGF-beta, IFN-γ, IL-10, IL-17, IL-6, IL-23, IL-22, IL-21, and MMPs. Another marker useful for selecting patients for treatment with a receptor agonist or antagonist is the level of CD73 in a tissue fluid of a patient. Inflammatory molecule levels can be measured by known methods including, but not limited to, quantitative RT-PCR and ELISA. Methods of identifying gene polymorphisms are well known in the art and include, but are not limited to, DNA sequencing and DNA microarrays.

Patients can also be monitored for the efficacy of a treatment with a receptor agonist or antagonist by screening the patients for levels of one or more biomarkers, including levels of one or more B7-H4 receptors of ligands thereof, during the course of treatment. The dosage, frequency, or a combination thereof can be increased or decreased accordingly. For example, the dosage or frequency of administration of a receptor agonist to a subject can be increased if the levels of one or more cytokines is elevated in the subject compared to levels in a control subject that does not have an inflammatory disorder, or the dosage or frequency of administration of a receptor agonist to a subject if the levels of one or more cytokines is reduced in the subject compared to levels in a control subject that does not have an inflammatory disorder.

### X. Methods of Manufacture

A. Methods for Producing Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acid encoding a variant polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. B7-H4 receptor-encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992.

Nucleic acids can be optimized for expression in the expression host of choice. Codons may be substituted with alternative codons encoding the same amino acid to account for differences in codon usage between the mammal from which the receptor nucleic acid sequence is derived and the expression host. In this manner, the nucleic acids may be synthesized using expression host-preferred codons.

B. Vectors and Host Cells

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences, are typically expressed as a fusion with the encoded polypeptide. Such tags can be inserted anywhere within the polypeptide including at either the carboxyl or amino terminus Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A. In one embodiment, a nucleic acid molecule encoding a B7-H4 receptor polypeptide is present in a vector containing nucleic acids that encode one or more domains of an Ig heavy chain constant region, preferably having an amino acid sequence corresponding to the hinge, C_{H2} and C_{H3} regions of a human immunoglobulin Cγ1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the B7-H4 receptor polypeptides described herein.

C. Methods for Producing Polypeptides

The disclosed polypeptides can be obtained by, for example, chemical synthesis or by recombinant production in a host cell. To recombinantly produce a polypeptide, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding a polypeptide. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well known in the art include, for example, Escherichia coli strains such as BL-21, and cultured mammalian cells such as CHO cells.

In eukaryotic host cells, a number of viral-based expression systems can be utilized to express polypeptides. Viral based expression systems are well known in the art and include, but are not limited to, baculoviral, SV40, retroviral, or vaccinia based viral vectors.

Mammalian cell lines that stably express polypeptides can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of variant polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, polypeptides can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Polypeptides can be isolated using, for example, chromatographic methods such as DEAE ion exchange, gel filtration, and hydroxylapatite chromatography. For example, a polypeptide in a cell culture supernatant or a cytoplasmic extract can be isolated using a protein G column. In some embodiments, variant polypeptides can be “engineered” to contain an amino acid sequence that allows the polypeptides to be captured onto an affinity matrix. For example, a tag such as c-myc, hemagglutinin, polyhistidine, or Flag (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase Immunoaffinity chromatography also can be used to purify polypeptides.

D. Methods for Making Antibodies

The immunogen used to generate the B7-H4 receptor antibody may be any immunogenic portion of a B7-H4 receptor. Preferred immunogens include all or a part of the extracellular domain of human B7-H4 receptor polypeptides, where these residues contain the post-translation modifications, such as glycosylation, found on the native polypeptide. Protein domains of neuropilins, plexins, semaphorins, as well as protein domains and predicted post-translation modifications thereof are provided in the sequences and tables provided above, and can be used to prepare immunogens for raising antibodies against a neuropilin, plexin, or semaphorin. Preferably, the immunogen includes the extracellular domain or immunogenic fragments thereof. Such peptides can be produced in a variety of ways known in the art, e.g., expression of cloned genes using conventional recombinant methods, synthesized peptide complexes, isolation from cells of origin, cell populations expressing high levels of B7-H4 receptor polypeptides. Antibodies for use in receptor occupancy assays include those that do not bind the receptor in the presence of the ligand, when compared to antibodies that block ligand binding—these can be selected by screening cells expressing the receptor in the presence/absence of ligand.

The antibodies may be polyclonal or monoclonal antibodies. The antibodies may be xenogeneic, allogeneic, syngeneic, or modified forms thereof, such as humanized or chimeric antibodies. The antibodies may also be antiidiotypic antibodies. Antibodies, as used herein, also includes antibody fragments including Fab and F(ab)₂ fragments, and antibodies produced as a single chain antibody or scFv instead of the normal multimeric structure. The antibodies may be an IgG such as IgG1, IgG2, IgG3 or IgG4; or IgM, IgA, IgE or IgD isotype. The constant domain of the antibody heavy chain maybe selected depending on the effector function desired. The light chain constant domain may be a kappa or lambda constant domain.

### XI. Methods for Identifying and Characterizing B7-H4 Receptors

Other receptors for B7-H4 can be identified by methods well known to those of skill in the art, including biochemical and functional genomic methods and based on structure function studies (e.g. crystal structures of ligands and predictive algorithms based on the 3-D structure).

The Examples below describes the identification and characterization of a neuropilin, a plexin, and complex thereof as receptors for B7-H4 and a semaphorin as a co-ligand thereof, using a receptor array screen adapted from Yao, et al., Immunity 34, 729-740, (2011). The screen can also be modified or adapted to identify other putative B7-H4 receptors. Briefly, plasmids encoding transmembrane genes are prepared and spotted into wells of multi-well tissue culture dishes. Cells, such as 293T cells, are added to each well and transiently transfected with the plasmids therein. The cells are given time to express the transfected plasmid.

Next, a bait molecule, such as B7-H4-Ig or another B7-H4 protein, such as any of those disclosed herein, alone or in combination with a B7-H4 receptor co-ligand is added to the wells. Preferably, the B7-H4-Ig or B7-H4 protein has a “tag” such as human IgG1 Fc, which can be used to identify the B7-H4-Ig or B7-H4 protein using a secondary antibody. A secondary antibody that can detect human Fc is added to the wells, and the plates are analyzed for B7-H4-Ig or B7-H4 protein binding to the surface of the transfected cells. In some embodiments, the Applied Biosystems 8200 cellular detection system and CDS 8200 software are used for the analysis.

In some embodiments, the cells used in the array are one or more of the cell types discussed below. In some embodiments, the one or more of the cell types below are transfected with plasmids expressing membrane proteins. In some embodiments, the cells are not transfected with plasmids encoding human transmembrane proteins. Therefore, the screen will detect interaction of B7-H4-Ig or B7-H4 protein with the endogenous receptor expressed by the cell.

### XII. Transgenic Animals

Transgenic non-human animals that do not express B7-H4 receptors or have reduced expression are useful in screening and testing. The endogenous B7-H4 receptor gene and alleles can be disrupted by inserting a genetic element into the gene to prevent expression. Preferably, the endogenous B7-H4 receptor gene is deleted using homologous recombination. In the case of carbohydrate or lipid receptors, genes involved in the biosynthetic pathways can be disrupted in order to target the receptor molecule. Representative non-human transgenic animals include mice or other rodents, sheep, goats, cows, pigs, and non-human primates.

The transgenic animals can be used to as research tools to study how B7-H4 receptors modulate the immune system, in particular how B7-H4 receptors function to suppress cellular immune responses. For example, the transgenic animals can be used to screen for compounds that mimic endogenous B7-H4 receptor biological activity or for compounds that interact with B7-H4 receptors.

### EXAMPLES

### Example 1

### B7-H4-Ig Bind to Neuropilin-1

Materials and Methods

Binding in Solution Using an ELISA-Type Method

Wells were coated with mouse or human Nrp-1, Sema6C, PlexinA4, or a combination thereof. The coated wells were treated with B7-H4-Ig-biotin, and bound protein was detected. Human B7-H4-Ig-biotin (1 μg/ml) for detecting.

The B7-H4-Ig used in the Examples has an amino acid sequence of (SEQ ID NO:23).

The Nrp-1 used in the Examples is recombinant human Neuropilin-1, R & D catalog number: 3870-N1; source=Mouse myeloma cell line, NS0 derived, Phe22Lys644, with a C-terminal, 6-His tag Accession # NP_001019799 (SEQ ID NO:63), and described at Gene ID: 8829, which is hereby incorporated by reference in its entirety.

The Sema6C used in the Examples is a recombinant Human Semaphorin 6C Fc (R & D systems Catalog No: 2219-S6-050). The protein has a domain structure, from N-terminal to C-terminal of

(Ala25-Val601) Accession #: NP_112175 (SEQ ID NO:40)—IEGRMD (SEQ ID NO:94)—Human IgG (Pro100-Lys330) Chimera Mouse myeloma cell line, NS0-derived.

The Plexin4A used in the Examples is recombinant human Plexin4A, R & D catalog number: 5856-PA; source=Chinese Hamster Ovary cell line, CHO derived Thr24Pro1237, with a C-terminal, 6 His tag Accession # Q9HCM2 (SEQ ID NO:2) and described at Gene ID: 91584 which is hereby incorporated by reference in its entirety.

Results

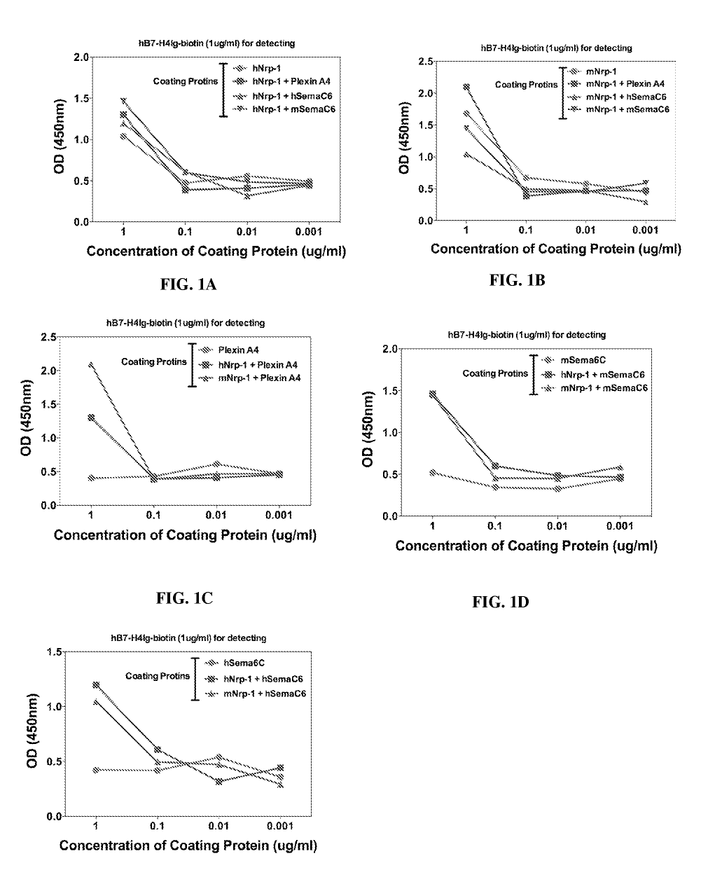

As shown in FIGS. 1A-1E, B7-H4-Ig binds Nrp-1, and the addition of PlexinA4 increases the binding. B7-H4-Ig does not appear to bind Sema6C or PlexinA4 in the absence of Nrp-1.

### Example 2

### Neuropilin-1 Bind B7-H4-Ig

Materials and Methods

Binding in Solution Using an ELISA-Type Method

Wells were coated with human Nrp-1 or B7-H4Ig. Detection was carried out with B7-H4Ig-biotin or Nrp-1-biotin, respectively, in the absence or presence of PlexinA4 and Sema6C.

Results

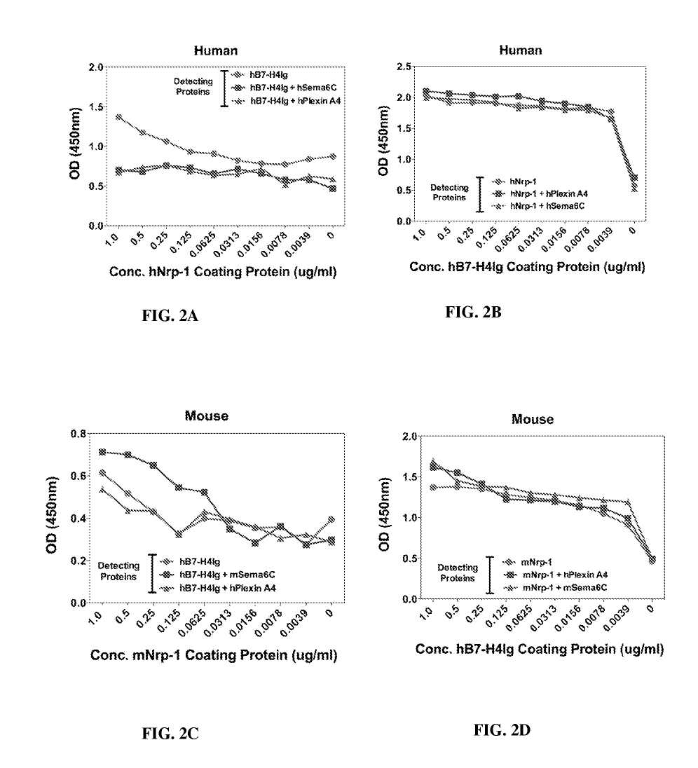

A shown in FIGS. 2A-2D, B7-H4-Ig binds Nrp-1, and the addition of PlexinA4 increases the binding.

### Example 3

### B7-H4 Binds to Sema3a

Materials and Methods

Over 4000 plasmids containing full length human transmembrane genes were transfected into 293T cells in the 384-well format by Lipofectamine 2000 (Yao, et al., Immunity, 34(5):729-40 (2011)). B7-H4-Ig was added as a bait together with anti-human Ig APC secondary antibody. The plates were read twenty-four hours after transfection by the Applied Biosystems 8200 cellular detection system and analyzed by CDS 8200 software. The Fc Receptor transfectants are used as positive controls.

The Sema3A used in the Examples has an amino acid sequence of (SEQ ID NO:62) (PreproTech Catalog Number 150-17).

Results

Cells transfected with Semaphorin 3A showed positive staining by B7-H4-Ig.

### Example 4

### B7-H4-Ig Binds to T Cells Containing Elevated Levels Sema3a, PlexinA4 and Neuropilin RNA

Materials and Methods

Mouse splenocytes from SJL/J mice were harvested and processed into a single cell suspension. After red blood cell lysis, total splenocytes were cultured at 5×10⁶ cells/well in a 24-well plate (1.5 mL per well) in the presence of anti-CD3 at 1 μg/ml in complete HL-1 medium. On day 3 of culture the cells are split into two tubes, i.e., one tube for B7-H4-Ig binding and one tube for Control Ig binding. The cells were treated with Cytochalasin D (final concentration of 1 μg/mL in culture medium), and incubated for 2 hr in a 37° C. incubator with CO₂. The cells were collected, washed and resuspended at 1×10⁶ cells/100 μL FACS staining buffer with Fc block and incubated at 4° C. for 20 min. The cells were then washed 3× in FACS staining buffer followed by staining with surface antibodies or isotype controls and incubated at 4° C. for 20 min in the dark. The cells were collected via centrifugation, washed 3× in FACS staining buffer, and resuspended at 1×10⁶ cells/100 μL FACS staining buffer plus biotinylated B7-H4-Ig or Control Ig (5 μg/1×10⁶ cells), and incubated at 4° C. for 30 min. The cells were collected, washed 3× in FACS staining buffer, and resuspended in 100 μL FACS staining buffer supplemented with PE-Streptavidin (eBioScience or BD, other color can be used) at 1:300 dilution, and incubated at 4° C. for 15 min in the dark. The cells were harvested, washed 3× in PBS, and resuspended at 1×10⁶ cells/100 μL of PBS plus LIVE/DEAD Fixable Aqua Dead Cell Staining Kit [Molecular Probes; L345957] at 1:1000 dilution followed by incubation at 4° C. for 30 min Cells were collected, washed 3× in PBS, and the labeled cells were resuspended at 20×10⁶ cells/mL in FACS staining buffer for FACS sorting.

From the B7-H4-Ig binding vial, both B7-H4-Ig binding negative cells and positive CD4+ T cells were sorted and collected. Both Control Ig binding negative cells and positive cells were also sorted and collected. All the cell samples were resuspended in RNAlater™ and shipped to Miltenyi Biotec on dry ice. RNA was isolated using standard RNA extraction protocols and passed Miltenyi RNA quality control tests. The RNA samples were then amplified and labeled using Miltenyi Biotec standard protocols followed by hybridization with Agilent Whole Mouse Genome Oligo Microarrays.

Results

Cells in the B7-H4-Ig binding peak and non-binding peak were sorted. Similar sorting was applied to Control Ig binding cells.

TABLE 18
Summary of Cells Collected from the FACSorter

| | Collected cell |
|---|---|
| Characteristics | number (×10⁶) |
| B7-H4-Ig Neg | 1.70 |
| B7-H4-Ig Pos | 0.18 |
| Ctrl Ig Neg | 2.43 |
| Ctrl Ig Pos | 0.21 |

RNA microarray analyses revealed that Sema3a, PlexinA4, Sema6c, Nrp-2, Nrp-1, DC-SIGNc and a few other DC-SIGN molecules (see Table 19) were present at increased levels in cells bound by B7-H4-Ig than in cells not bound by B7-H4-Ig.

TABLE 19
RNA Microarray Analyses of FACS-sorted Cells

| | B7-H4Ig binding− vs. | Ctrl Ig binding+ vs. | Ctrl Ig binding− vs. | Ctrl Ig binding− vs. B7- | |
|---|---|---|---|---|---|
| | B7-H4Ig binding+ | B7-H4Ig binding+ | Ctrl Ig binding+ | H4Ig binding− | Fold Change |
| Neuropilin 1 | 1.693 | −1.007 | 1.340 | 1.035 | +10 |
| Neuropilin 2 | 8.063 | 1.412 | 6.780 | −1.034 | +5 |
| PlexinA4 | 3.664 | 1.807 | 1.465 | 1.000 | +2 |
| Sema6c | 3.481 | 2.239 | 1.283 | −1.269 | +1 |
| Sema3a | 6.311 | 2.961 | 1.424 | 1.008 | 0.000 |
| Sema4a | 1.058 | 1.012 | −1.016 | −1.096 | −1.000 |
| LAIR1 | −6.209 | 1.013 | −2.291 | −1.173 | −2.000 |
| DC-SIGNa | −1.011 | 1.027 | 1.176 | −1.090 | −5.000 |
| DC-SIGNb | 1.837 | −1.000 | 1.421 | −1.002 | −10.000 |
| DC-SIGNc | 39. [symbol] 24 | 15.472 | 1.322 | 1.006 | |
| DC-SIGNd | 1.263 | 2.518 | −2.554 | 1.252 | |
| DC-SIGNe | 1.755 | 1.007 | 1.319 | 1.001 | |
| DC-SIGNf | 1.192 | 1.001 | −1.062 | 1.042 | |
| DC-SIGNg | 1.813 | 1.007 | 1.384 | 1.003 | |
| S1P1 | −1.602 | −1.474 | 1.061 | 1.125 | |
| c-Kit | −3.594 | 1.303 | −5.685 | 1.508 | |
| Tiam 1 | −2.894 | 1.603 | −6.023 | 1.187 | |

[symbol]

indicates data missing or illegible when filed

### Example 5

### Sema3a Binds to B7-H4-Ig

Materials and Methods

Sema3a recombinant protein (Peprotech) reconstituted in PBS at 1.0 μg/mL was immobilized on a 96-well ELISA plate at 4 degrees C. overnight. The plate was washed and blocked with PBS containing 10% FBS for 1 hour at room temperature (RT). A titration of B7-DCIg (fusion protein control), Control Ig (anti-RSV, isotype control), or B7-H4-Ig from 0.01 μg/ml to 1000 μg/ml were added to the plate for 2 hours at RT to evaluate binding activities. The plate was washed and anti-human IgG Fc HRP was added to the plate for 1 hour at RT. The plate was washed and TMB substrate was added for 30 mins. The reaction was stopped with 1N sulfuric acid. The plate was read with Perkin Elmer Envision 2104 Reader.

Results

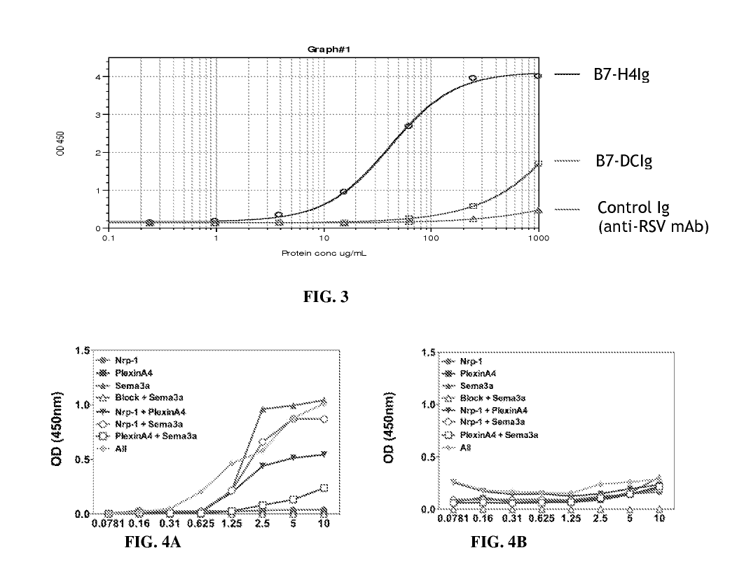

As shown in FIG. 3, Sema3a binds to B7-H4-Ig. No or low binding of Sema3a to Control Ig or B7-DCIg is observed.

### Example 6

### B7-H4-Ig interacts with Sema3a/Nrp-1/PlexinA4 Complexes

Materials and Methods

96-well ELISA plates were coated with recombinant human Nrp-1 (R&D Systems), PlexinA4 (R&D Systems), or Sema3a (Peprotech)(all at 1 μg/mL) or both Nrp-1+PlexinA4 overnight at 4° C. Following incubation the plates were washed 4× with 1×PBS, plates were blocked with blocking buffer (PBS+5% BSA) for 1 hour at 37° C., and washed 4× with 1×PBS. The wells coated with Nrp-1 (——), PlexinA4a (—▪—), Sema3a (—▴—), and Nrp-1+PlexinA4 (—▾—) only were further incubated with PBS+5% BSA alone. For wells that received a combination of Block+Sema3a (—Δ—), Nrp-1+Sema3a (—◯—), PlexinA4+Sema3a (—□—), or Nrp-1+PlexinA4+Sema3a (—♦—); soluble Sema3a (1 μg/mL) in PBS+5% BSA was added to the respective wells and incubated at 37° C. for 2 hours.

Following the incubation, the wells were washed 4× with 1×PBS, and biotinylated B7-H4-Ig or B7-DCIg (fusion protein control) was added to the wells at 10, 5, 2.5, 1.25, 0.625, 0.313, 0.16, 0.08 μg/mL in PBS+5% BSA, and incubated overnight at 4° C. Following the incubation, the wells were washed 4× with 1×PBS, streptavidin-HPR diluted 1:1000 in PBS+5% BSA was then added, and the plate was incubated for 30 minutes at 37° C. The wells were then washed 6× with 1×PBS and the ELISA plate was developed with TMB substrate (BioFX), and read on SpectraMax M2 (Molecular Devices).

Results

B7-DC-Ig, the fusion protein control, did not bind to Nrp-1, PlexinA4, Sema3a or any combination of these proteins. B7-H4-Ig very weakly bound to Nrp-1 or PlexinA4 alone (FIG. 4B). In contrast, B7-H4-Ig bound strongly to recombinant Sema3a (FIG. 4A—solid triangle). The Block+Sema3a wells (FIGS. 4A and 4B—open triangle) served as a set of negative control wells, and controlled for incomplete blocking of the Nrp-1+Sema3a, PlexinA4+Sema3a, and Nrp-1+PlexinA4+Sema3a, as well as any non-specific Sema3a interaction with BSA.

The data show that in wells incubated with PBS+5% BSA followed by recombinant Sema3a incubation, B7-H4-Ig did not bind. Incubation of Sema3a in wells pre-coated with Nrp-1 (FIGS. 4A and 4B—open circles), PlexinA4 (FIGS. 4A and 4B—open squares), or Nrp-1+PlexinA4 (FIGS. 4A and 4B—diamonds) allowed for greater B7-H4-Ig binding. Previous studies show that Sema3a binds to both Nrp-1 and PlexinA4. The data presented in FIGS. 4A and 19B show that B7-H4-Ig binds to Nrp-1/Sema3a, PlexinA4/Sema3a, and possibly Nrp-1/PlexinA4/Sema3a complexes.

### Example 7

### Nrp-1 is Important for B7-H4-Ig-Induced Immune Regulatory Function of T Cells In Vitro

Materials and Methods

Spleens from wildtype C57BL/6 mice or mice with Nrp-1 conditionally knocked out in FoxP3 expressing cells (Nrp-1^{-/-}) on a C57BL/6 background were harvested and processed into single cell suspensions. After lysis of red blood cells, the splenotypes (5×10⁵) were added to 96 well, flat-bottom wells and activated with 1 μg/mL of anti-CD3 together with 0, 0.16, 0.32, 1.25, 2.5, 5 or 10 μg/mL of B7-H4-Ig. At 24 hours post culture initiation, wells were pulsed with [³H]-thymidine (1 μCi/well) to assess cellular proliferation and cultures were harvested at 72 hours. Supernatants were also collected at 72 hours post culture initiation to determine the levels of secreted cytokines via LiquiChip analysis.

Results

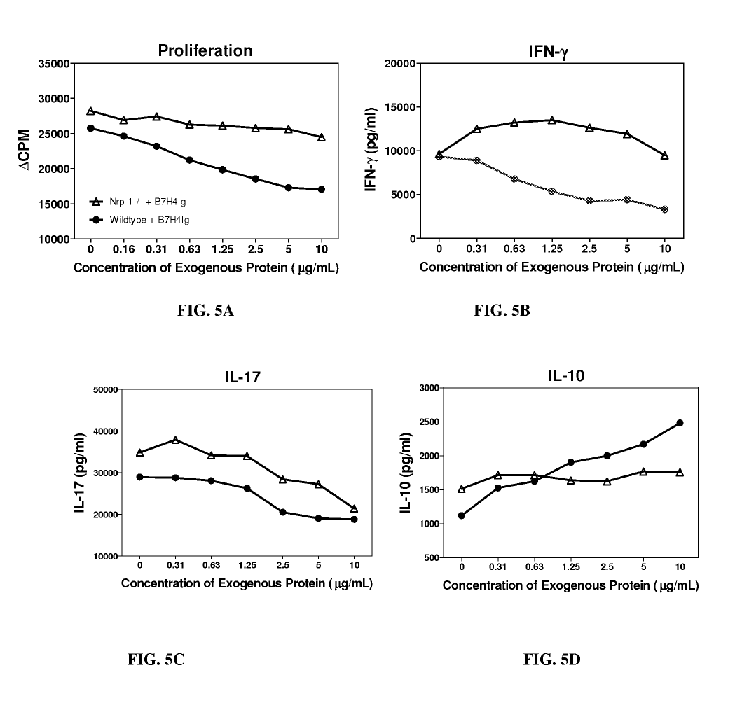

FIGS. 5A-5D show that B7-H4-Ig blocks anti-CD3-induced proliferation/activation in wildtype T cells. B7-H4-Ig decreased IFN-gamma and IL-17 production, as well as increased the level of IL-10 secreted into the culture supernatant of wildtype cells in a dose-dependent fashion (FIGS. 5A-5D—closed circles). However, the immunoregulatory effects of B7-H4-Ig (decreased proliferation and IFN-γ production and enhanced IL-10 production) were not seen in cells derived from the Nrp-1^{-/-} mice (FIGS. 5A-5D—open triangles) with the exception that IL-17 production was decreased in cells from both wildtype and Nrp-1^{-/-} mice in the presence of B7-H4-Ig.

Wildtype C57BL/6 or Nrp-1^{-/-} splenocytes were also cultured at 5×10⁶ cells/well in a final volume of 1.5 ml in a 24-well plate in the presence of anti-CD3 (1 μg/mL) plus Control Ig or B7-H4-Ig (10 μg/mL), and culture supernatants were collected on Day 3 of culture. As shown in FIG. 6, B7-H4-Ig treatment decreased the level of IFN-γ, GM-CSF and IL-17 secreted and increased the level of IL-10 secreted by wildtype T cells (FIG. 6, B bars vs. A bars). However, the immunomodulatory function of B7-H4-Ig treatment is lost in cultures containing T cells from Nrp-1−/− Treg knockouts (FIG. 6, D bars vs. C bars).

### Example 8

### Nrp-1 is Important for B7-H4-Ig-Induced Immune Regulatory Function of Non-Obese Diabetic (NOD) Mouse T Cells In Vitro

Materials and Methods

Spleens from wildtype NOD mice or Nrp-1 conditional knockout in FoxP3 expressing cells (Nrp-1^{-/-}) mice on a NOD background were harvested and separated to single cell suspension. After lysis of red blood cells, the splenotypes (5×10⁵) were added to 96 well, flat-bottom wells and activated with 1 μg/mL of anti-CD3 together with 0, 0.16, 0.32, 1.25, 2.5, 5 or 10 μg/mL of B7-H4-Ig. Culture supernatants were collected at 72 hours post culture initiation and the level of secreted cytokines determined via LiquiChip analysis.

Results

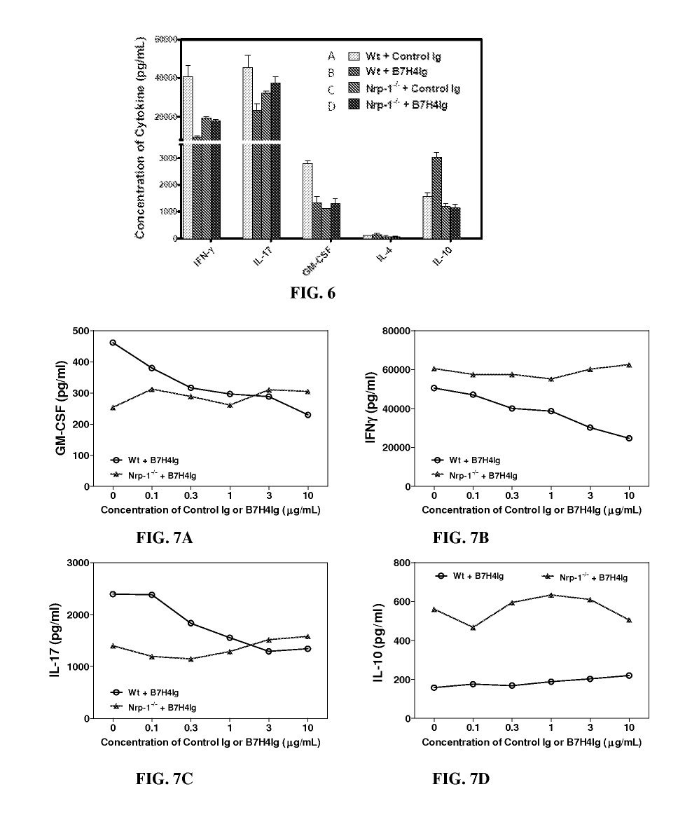

FIGS. 7A-7D show that B7-H4-Ig treatment decreased the level of secreted GM-CSF, IFN-γ, and IL-17 production and slightly increased the level of IL-10 production by T cells isolated from NOD mice. However, in cultures containing the Nrp-1^{-/-} cells isolated from NOD mice, B7-H4-Ig treatment did not decrease the secreted levels of IFN-gamma, GM-CSF and IL-17 or appreciably affect the secreted level of IL-10.

### Example 9

### Nrp-1 and PlexinA4 Partially Blocks B7-4 Ig Function In Vitro

Materials and Methods

Spleens from wildtype SJL/J mice were harvested and process into single cell suspensions. After lysis of red blood cells, the splenocytes (5×10⁵) were added to 96 well, flat-bottom wells and activated with 1 μg/mL of anti-CD3 together with 0, 0.313, 1.25, 2.5, 5 or 10 μg/mL of B7-H4-Ig. Control Ig, recombinant human Nrp-1, recombinant human PlexinA4, or serial diluted B7-H4-Ig+2.5 μg/mL of Nrp-1 (Ig fusion; R&D Systems) or PlexinA4 (Ig fusion; R&D Systems). At 24 hours post culture initiation, the cultures were pulsed with [³H]-thymidine (1 μCi/well) and cultures were harvested at 72 hours to assess the level of cellular proliferation.

Results

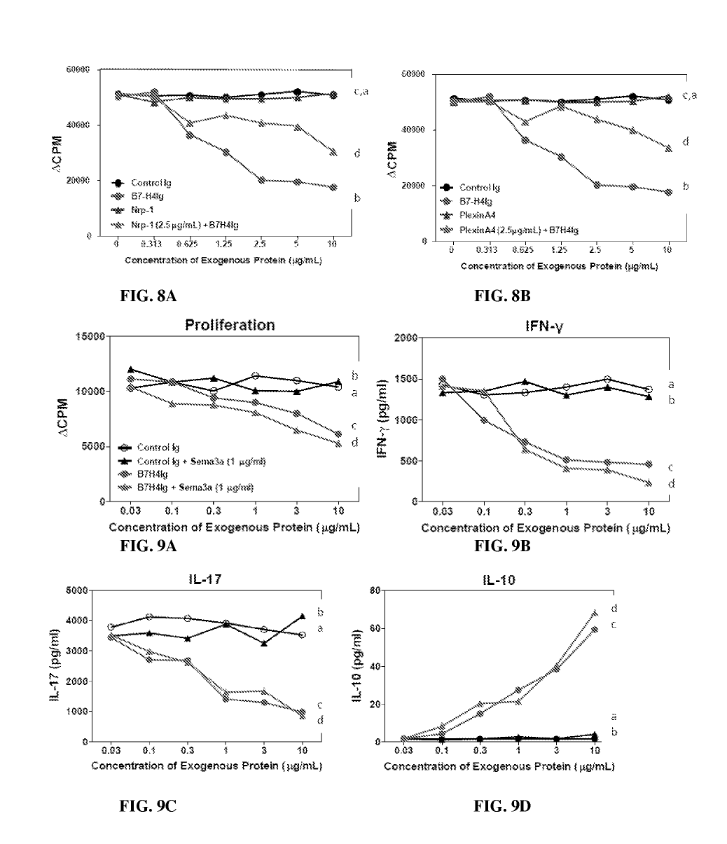

FIGS. 8A-8D show that the addition of either recombinant Nrp-1 or recombinant PlexinA4 alone did not alter the level of anti-CD3-induced T cell proliferation (FIGS. 8A-8B). In contrast, the treatment of the cultures with B7-H4-Ig decreased the level of anti-CD3-induced proliferation in a concentration-dependent manner (FIGS. 8A-8B). Furthermore, the co-treatment of cultures with recombinant human Nrp-1 (2.5 μg/mL) (FIG. 8A) or recombinant human PlexinA4 (2.5 μg/mL) (FIG. 8B) partially blocked the immunosuppression function of B7-H4-Ig (FIG. 8A-8B).

### Example 10

### Soluble Sema3a does not Affect B7-H4-Ig-Induced Immunoregulatory Function In Vitro

Materials and Methods

Spleens from wildtype SJL/J mice were harvested and processed into single cell suspensions. After lysis of red blood cells, the splenocytes (5×10⁵) were added to 96 well, flat-bottom wells and activated with 1 μg/mL of anti-CD3 together with 0.03, 0.1, 0.3, 1, 3, or 10 μg/mL of Control Ig or B7-H4-Ig in the absence or presence of recombinant human Sema3a (1 μg/mL). At 24 hours post culture initiation, wells were pulsed with [³H]-thymidine (1 μci/well) and cultures were harvested at 72 hours to assess cellular proliferation. Culture supernatants were collected at 72 hours post culture initiation and the level of secreted cytokine determined via LiquiChip analysis.

Results

FIGS. 9A-9D show that the addition of B7-H4-Ig to the cultures decreases the level of anti-CD3-induced T cell proliferation (24A), decreases the level of secreted IFN-gamma (24B) and IL-17 (24C), and increases the level of IL-10 (24D) secreted in a concentration-dependent manner. Addition of soluble recombinant human Sema3a did not alter the level of cellular proliferation or the level of cytokine created beyond that altered by treatment with Control Ig or B7-H4-Ig alone.

### Example 11

### Sema3a is Required for B7-H4-Ig-Induced Immunoregulatory Function In Vitro

Materials and Methods

Spleens from wildtype C3H/HeJ mice or Sema3a loss-of-function mice (Sema3^{-/-}, at Sema3a^{m808Ddg}, the Jackson Laboratory) were harvested and processed into single cell suspensions. After lysis of red blood cells, the splenotypes (5×10⁵) were added to 96 well, flat-bottom wells and activated with 1 μg/mL of anti-CD3 together with 0.1, 1, or 10 μg/mL of Control Ig or B7-H4-Ig in the absence or presence of recombinant human Sema3a (1 μg/mL). Culture supernatants were collected at 72 hours post culture initiation and the level of secreted cytokine determined via LiquiChip analysis.

Results

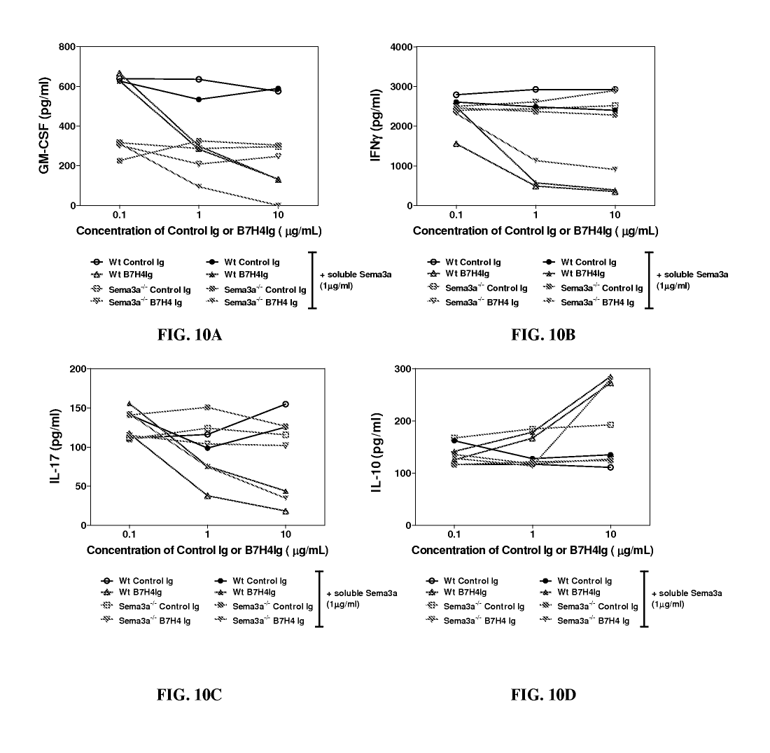

FIGS. 10A-10D show that the addition of B7-H4-Ig to the wildtype C3H/HeJ cultures decreases the level of anti-CD3-induced T cell secretion of GM-CSF, IFN-gamma, and IL-17, and increases the level of IL-10 secreted in a concentration-dependent manner. The addition of exogenous recombinant human Sema3a (1 μg/mL) did not alter B7-H4-Ig function. In contrast, the treatment of Sema3a^{-/-} cultures with increasing concentrations of B7-H4-Ig did not alter the level of cytokines secreted, but the addition of exogenous recombinant human Sema3a (1 μg/mL) promoted the recovery of B7-H4-Ig function. i.e., a concentration-dependent decrease in the level of secreted GM-CSF, IFN-gamma, and IL-17, and increases the level of IL-10 secreted. These data demonstrate that Sema3a is important for B7-H4-Ig function in culture.

### Example 12

### B7-H4-Ig Binding on Activated CD4+ T Cells Correlates with Sema3a Positivity

Materials and Methods

Splenocytes were harvested from SJL/J mice and processed into single cell suspensions. After red blood cell lysis, cells were treated with Cytochalasin D (final concentration of 1 μg/mL in culture medium), and incubated for 2 hr at 37° C. incubator with CO₂. The cells were collected, washed and resuspended at 1×10⁶ cells/100 μL FACS staining buffer with Fc block and incubated at 4° C. for 20 min. The cells were then washed 3× in FACS staining buffer followed by staining in the presence of antibodies to surface proteins or isotype controls and incubated at 4° C. for 20 min in the dark. The cells were collected via centrifugation, washed 3× in FACS staining buffer, and resuspended at 1×10⁶ cells/100 μL FACS staining buffer plus biotinylated B7-H4-Ig or Control Ig (5 μg/1×10⁶ cells), and incubated at 4° C. for 30 min. The cells were collected, washed 3× in FACS staining buffer, and resuspended in 100 μL FACS staining buffer supplemented with PE-Streptavidin (eBioScience or BD, other color can be used) at 1:300 dilution, and incubate at 4° C. for 15 min in the dark. The cells were harvested, washed 3× in PBS, and resuspended at 1×10⁶ cells/100 μL of PBS plus LIVE/DEAD Fixable Aqua Dead Cell Staining Kit [Molecular Probes; L345957] at 1:1000 dilution followed by incubation at 4° C. for 30 min Cells were collected, washed 3× in PBS, and the labeled cells were resuspended at 20×10⁶ cells/mL in FACS staining buffer for flow cytometric analysis.

Results

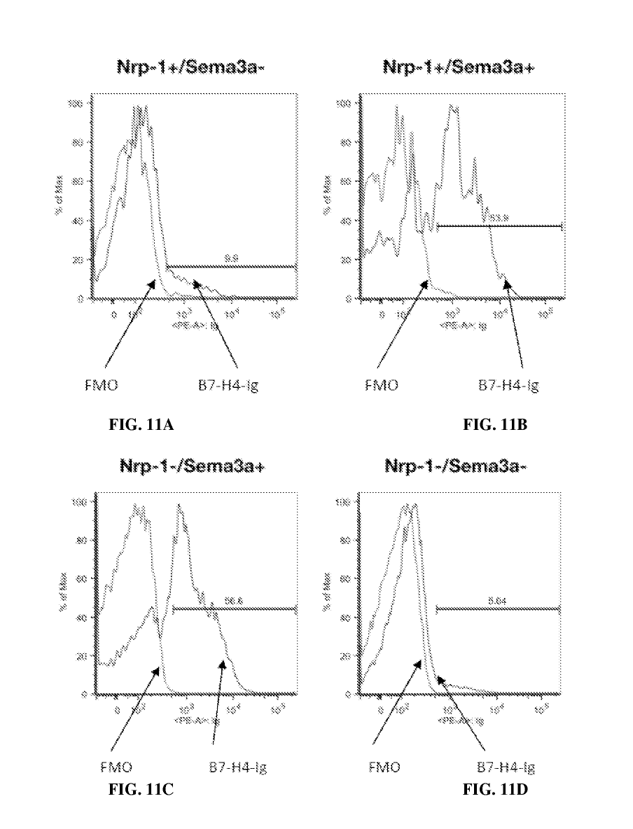

The results of the experiment, shown in FIGS. 11A-11D, indicate that CD4+T cells that are Sema3a+ bind B7-H4-Ig.

### Example 13

### B7-H4-Ig Binding CD4+ T Cells from PLP₁₃₉₋₁₅₁ Reactivation Cultures are Sema3a+

Materials and Methods

SJL/J mice were primed with PLP₁₃₉₋₁₅₁/CFA and on Day +8 post priming the draining lymph nodes are harvested and processed into single cell suspensions. The cells were cultured at 5×10⁶ cells/well in a 24-well plate (1.5 mL per well) in the presence of PLP₁₃₉₋₁₅₁ peptide (10 μg/ml) in complete HL-1 medium. On day 3 of culture the cells were treated with Cytochalasin D (final concentration of 1 μg/mL in culture medium), and incubated for 2 hr at 37° C. incubator with CO₂. The cells were collected, washed and resuspended at 1×10⁶ cells/100 μL FACS staining buffer with Fc block and incubated at 4° C. for 20 min. The cells were then washed 3× in FACS staining buffer followed by staining in the presence of cell surface antibodies or isotype controls and incubated at 4° C. for 20 min in the dark. The cells were collected via centrifugation, washed 3× in FACS staining buffer, and resuspended at 1×10⁶ cells/100 μL FACS staining buffer plus biotinylated B7-H4-Ig or Control Ig (5 μg/1×10⁶ cells), and incubated at 4° C. for 30 min. The cells were collected, washed 3× in FACS staining buffer, and resuspended in 100 μL FACS staining buffer supplemented with PE-Streptavidin (eBioScience or BD) at a 1:300 dilution, and incubated at 4° C. for 15 min in the dark. The cells were harvested, washed 3× in PBS, and resuspended at 1×10⁶ cells/100 μL of PBS plus LIVE/DEAD Fixable Aqua Dead Cell Staining Kit [Molecular Probes; L345957] at 1:1000 dilution followed by incubation at 4° C. for 30 min Cells were collected, washed 3× in PBS, and the labeled cells were resuspended at 20×10⁶ cells/mL in FACS staining buffer for flow cytometric analysis.

Results

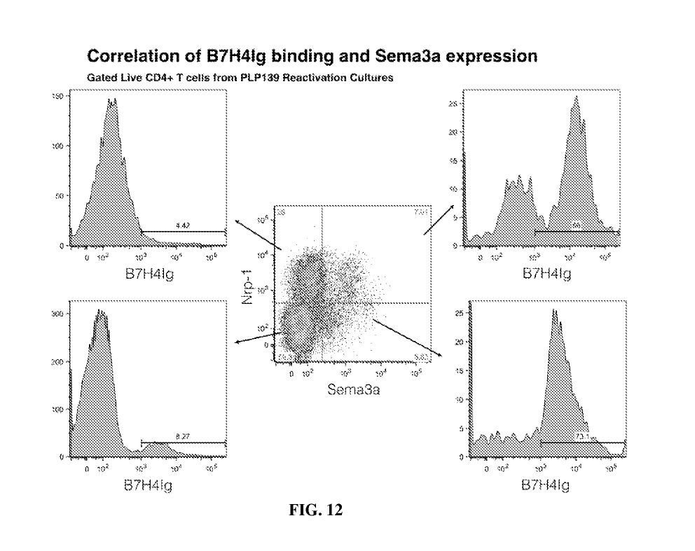

The results of the experiment, shown in FIG. 12, indicate that the majority of the Sema3a+CD4+ T cells from PLP₁₃₉₋₁₅₁ reaction cultures bind B7-H4-Ig.

### Example 14

### B7-H4-Ig Binds to Sema3a+293 Cells

Materials and Methods

293 cells were cultured at 1×10⁶ cells/well in a 24-well plate (1 mL per well) overnight in complete RPMI medium. The next morning the cells were treated with Cytochalasin D (final concentration of 1 μg/mL in culture medium), and incubated for 2 hr at 37° C. incubator with CO₂. The cells were collected, washed and resuspended at 1×10⁶ cells/100 μL FACS staining buffer alone or FACS staining buffer containing recombinant human Sema3a (1 μg/ml), and incubated at 4° C. for 20 min. The cells were collected, washed and resuspended at 1×10⁶ cells/100 μL FACS staining buffer with Fc block and incubated at 4° C. for 20 min. The cells were then washed 3× in FACS staining buffer followed by staining in the presence of cell surface antibodies or isotype controls and incubated at 4° C. for 20 min in the dark. The cells were collected via centrifugation, washed 3× in FACS staining buffer, and resuspended at 1×10⁶ cells/100 μL FACS staining buffer plus biotinylated B7-H4-Ig or Control Ig (5 μg/1×10⁶ cells), and incubated at 4° C. for 30 min. The cells were collected, washed 3× in FACS staining buffer, and resuspended in 100 μL FACS staining buffer supplemented with PE-Streptavidin (eBioScience or BD, other color can be used) at 1:300 dilution, and incubated at 4° C. for 15 min in the dark. The cells were harvested, washed 3× in PBS, and resuspended at 1×10⁶ cells/100 μL of PBS plus LIVE/DEAD Fixable Aqua Dead Cell Staining Kit [Molecular Probes; L345957] at 1:1000 dilution followed by incubation at 4° C. for 30 min Cells were collected, washed 3× in PBS, and the labeled cells were resuspended at 20×10⁶ cells/mL in FACS staining buffer for flow cytometric analysis.

Results

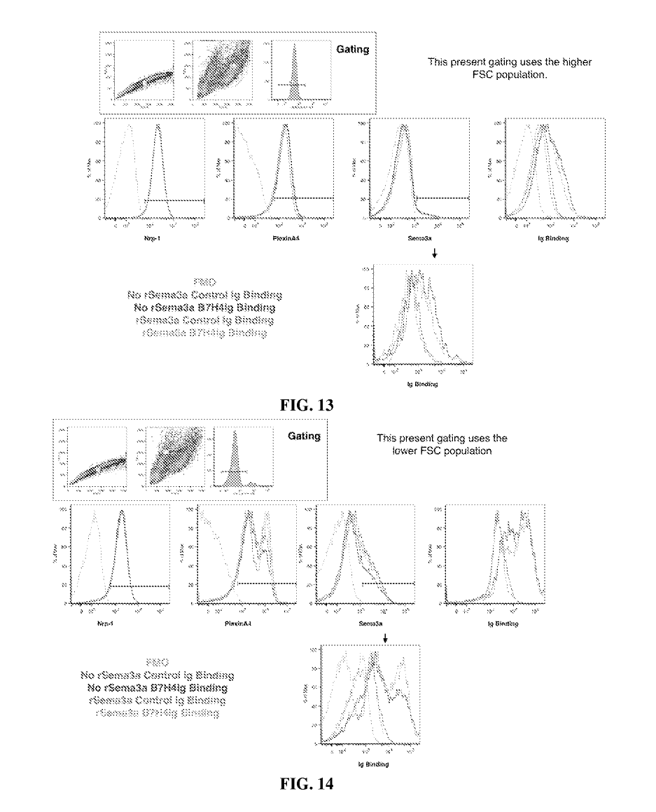

The results show that there are two live populations of 293 cells following culture, i.e., a larger FSC population and a smaller FSC population. The smaller FSC population of 293 cells has a higher level of PlexinA4 and Sema3a present on the cell surface (compare FIG. 14 (smaller FSC population) to FIG. 13 (larger FSC population). This same population of cells also binds B7-H4-Ig to a much greater extent as compared to the larger FSC population of 293 cells. However, the addition of a recombinant human Sema3a binding step did not allow for greater Sema3a+ being bound to the cell surface, as compared to cells that did not receive exogenous recombinant human Sema3a. Similar to the data present above for the CD4+ T cells, binding of B7-H4-Ig correlated with Sema3a being present on the cell surface (FIGS. 13-14).

### Example 15

### Addition of Recombinant Sema3a or B7-H4-Ig Alters 293 Cell Proliferation

Materials and Methods

293 cells were cultured at 50×10⁵ cells/well in a flat-bottom 96-well plate in a final volume of 200 μL in complete RPMI containing 1 μCi of tritiated thymidine. In triplicate wells the 293 cells were treated with recombinant human Sema3a at 0, 0.3, 1, 3, or 10 μg/ml in the presence of B7-H4-Ig at 0, 1, 5, or 10 μg/ml. On Day 3 of culture, wells were harvested and the amount of tritiated thymidine incorporation was assessed.

Results

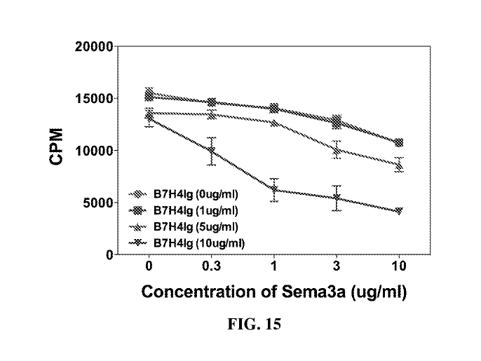

The results shown in FIG. 15 indicates that the addition of exogenous recombinant human Sema3a is able to decrease the level of 293 cell proliferation in a concentration-dependent manner. Similarly, the addition of soluble B7-H4-Ig was able to further decrease the level of 293 cell proliferation in a concentration-dependent manner, with the maximal reduction of 293 cells proliferation in the presence of 10 μg/ml exogenous recombinant human Sema3a and 10 μg/ml B7-H4-Ig.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for selecting subjects for treatment with a B7-H4-Ig fusion protein comprising determining the level of B7-H4 receptor protein on immune cells of the subject and selecting the subject for treatment with B7-H4-Ig if the level of B7-H4 receptor protein.

2. The method of claim 1, wherein the B7-H4 receptor protein comprises NRP-1, Sem3A, plexin or a combination thereof.

3. The method of claim 1 wherein the subject has an inflammatory or autoimmune disease or disorder.

4. The method of any one of claims 1-3 wherein the control is the level of neuropilin expressed on immune cells of a subject without an inflammatory or autoimmune disease or disorder, or an average from subjects without an inflammatory or autoimmune disease or disorder.

5. The method of any one of claims 1-4 wherein the fusion protein comprises the extracellular domain of B7-H4.

6. The method of any one of claims 1-5 wherein the fusion protein comprises the amino acid sequence of SEQ ID NO:23 or SEQ ID NO:25.

7. A method of diagnosing a subject with an immunological disorder comprising determining the level of B7-H4 receptor protein expressed on immune cells of the subject wherein under-expression of B7-H4 receptor protein relative to a control is indicative of an immunological disorder.

8. The method of claim 7, wherein the B7-H4 receptor protein comprises NRP-1, Sem3A, plexin or a combination thereof.

9. The method of claim 8, wherein the immunological disorder is selected from the group consisting of inflammatory disorder and autoimmune disorder.

10. A method for selecting a patient for treatment of an immune disorder comprising determining the levels of soluble B7-H4 to the B7-H4 receptor in a sample obtained from the subject, selecting the patient for treatment when high levels of binding between soluble B7-H4 and B7-H4 receptor are detected.

11. The method of claim 10, wherein the immune disorder is an autoimmune disorder or inflammation.

12. A pharmaceutical composition comprising one or more antagonists of neuropilin, wherein the antagonist blocks, inhibits, or otherwise reduces binding or signal transduction between B7-H4 polypeptide and neuropilin.

13. The pharmaceutical composition of claim 12, wherein the antagonist blocks, inhibits, or otherwise reduces binding or signal transduction between transmembrane B7-H4 and neuropilin.

14. The pharmaceutical composition of any one of claims 12-13 wherein the antagonist comprises a soluble neuropilin polypeptide.

15. The pharmaceutical composition of any one of claims 12-13 wherein the antagonist is a soluble fusion protein comprising a first domain comprising a neuropilin polypeptide and a second domain.

16. The pharmaceutical composition of claim 15 wherein the second domain comprises an Ig Fc region.

17. The pharmaceutical composition of any of claims 15-16 wherein the soluble neuropilin polypeptide comprises the extracellular domain of SEQ ID NO:1 or functional fragment or variant thereof comprising at least 80% sequence identity to the extracellular domain of SEQ ID NO:1.

18. The pharmaceutical composition of any of claim 17 wherein soluble neuropilin is complexed with a soluble plexin polypeptide or a fusion protein thereof.

19. The pharmaceutical composition of claim 18 wherein the plexin is Plexin4A.

20. The pharmaceutical composition of claim 19 wherein the soluble plexin comprises or consists of the extracellular domain of SEQ ID NO:2 or functional fragment or variant thereof comprising at least 80% sequence identity to the extracellular domain of SEQ ID NO:2.

21. The pharmaceutical composition of claim 12 wherein the antagonist is a soluble B7-H4 polypeptide.

22. The pharmaceutical composition of claim 12 wherein the soluble B7-H4 polypeptide consists of the extracellular domain of B7-H4 or a fragment or variant thereof.

23. The pharmaceutical composition of claims 16-17 wherein the antagonist is an anti-B7-H4 antibody or an antigen binding fragment thereof; an anti-B7-H4 receptor antibody or an antigen binding fragment thereof; a bi-specific antibody that targets B7-H4 and the B7-H4 receptor; or a combination thereof and reduces, inhibits or blocks signal transduction through the B7-H4 receptor.

24. The pharmaceutical composition of claim 23 wherein the antibody or antigen binding fragment thereof binds to the extracellular domain of SEQ ID NO:1 or a functional fragment or variant thereof comprising 80% sequence identity to the extracellular domain of SEQ ID NO:1.

25. The pharmaceutical composition of claim 24 wherein the extracellular domain comprises amino acids 22-856 of SEQ ID NO:1, or a functional fragment thereof.

26. The pharmaceutical composition of claim 23 wherein the antibody or antigen binding fragment thereof binds to the extracellular domain of SEQ ID NO:2 or a functional fragment or variant thereof comprising at least 80% sequence identity to the extracellular domain of SEQ ID NO:2.

27. The pharmaceutical composition of claim 26 wherein the extracellular domain comprises amino acids 24-1894 of SEQ ID NO:2.

28. The pharmaceutical composition of claim 23 wherein the antibody or antigen binding fragment thereof binds to SEQ ID NO:40 or a functional fragment or variant thereof comprising at least 80% sequence identity to SEQ ID NO:40.

29. The pharmaceutical composition of claim 23 wherein the antibody or antigen binding fragment thereof binds to SEQ ID NO:43 or a functional fragment or variant thereof comprising at least 80% sequence identity to SEQ ID NO:43.

30. A pharmaceutical composition comprising one or more antagonists of a B7-H4 receptor comprising a neuropilin,
wherein at least one of the antagonists is an inhibitory nucleic acid that reduces expression of the neuropilin.

31. The pharmaceutical composition of claim 30 further comprising an inhibitory nucleic acid that reduces expression of a plexin, an inhibitory nucleic acid that reduces expression of a semphorin, or a combination thereof.

32. The pharmaceutical composition of claim 30 or 31 wherein the neuropilin is NRP-1.

33. The pharmaceutical composition of claim 31 wherein the plexin is Plexin4A.

34. The pharmaceutical composition of any of claims 12-33 wherein the antagonist is present in an amount effective to inhibit or reduce an immune inhibitory response.

35. The pharmaceutical composition of any of claims 12-34 wherein the antagonist is present in an amount effective to induce, increase, or enhance an immune stimulatory response.

36. The pharmaceutical composition of any of claims 12-35 wherein the composition comprises two more antagonists.

37. The pharmaceutical composition of any of claims 12-36 wherein the antagonist is in a dosage effective to break Treg mediated immune tolerance, increase a T cell response, increase proliferation of T cells, differentiation or effector function of T cells or survival of T cells.

38. The pharmaceutical composition of any of claims 12-37 wherein the antagonist is in a dosage effective to reduce or inhibit a regulatory T cell response, proliferation of regulatory T cells, differentiation or effector function of regulatory T cells or survival of regulatory T cells.

39. An immunogenic composition comprising the pharmaceutical composition of any of claims 12-38 and an antigen.

40. A method for treating diseases associated with elevated levels of B7-H4, in a subject comprising administering to the subject the pharmaceutical composition of any of claims 12-39.

41. The method of claim 40, wherein the disease is cancer or an infectious disease.

42. The method of claim 41 wherein the cancer is selected from the group consisting of bladder, brain, breast, cervical, colo-rectal, esophageal, kidney, liver, lung, nasopharangeal, pancreatic, prostate, skin, stomach, uterine, ovarian, and testicular cancers.

43. A method for enhancing Th1 and Th17 responses in a subject, the method comprising administering to the subject the pharmaceutical composition of any of claims 12-37.

44. A method for treating elevated expression of B7-H4, in a subject comprising administering to the subject the pharmaceutical composition of any of claims 12-37.

45. A method for inducing, enhancing, maintaining or prolonging an immune response in an individual comprising administering to the individual the pharmaceutical composition of any of claims 12-37.

46. The method of claim 45 wherein the individual has an infection or cancer.

47. The method of claim 46 wherein the cancer is selected from the group consisting of bladder, brain, breast, cervical, colo-rectal, esophageal, kidney, liver, lung, nasopharangeal, pancreatic, prostate, skin, stomach, uterine, ovarian, and testicular cancers.

48. The method of claim 46 wherein the infection is due to a virus, bacteria, fungus, or protozoa.

49. A method of treating cancer in a subject comprising administering to the subject an effective amount of the pharmaceutical composition of any of claims 5-37 to increase or induce apoptosis of tumor cells, reduce or inhibit tumor cell proliferation, reduce or inhibit tumor cell migration, reduce or inhibit the Erk1/2 pathway in tumor cells, or a combination thereof.

50. A method of increasing or inducing apoptosis of tumor cells comprising administering to a subject an effective amount of the pharmaceutical composition of any of claims 12-37 to increase or induce apoptosis of tumor cells in the subject compared to a control.

51. A method of reducing or inhibiting tumor cell proliferation comprising administering to a subject an effective amount of the pharmaceutical composition of any of claims 12-37 to reduce or inhibit tumor cell proliferation in the subject compared to a control.

52. A method of reducing or inhibiting tumor cell migration comprising administering to a subject an effective amount of the pharmaceutical composition of any of claims 12-37 to reduce or inhibit tumor cell migration in the subject compared to a control.

53. A method of reducing or inhibiting the Erk1/2 pathway in tumor cells comprising administering to a subject an effective amount of the pharmaceutical composition of any of claims 12-37 to reduce or inhibit the Erk1/2 pathway in tumor cells in the subject compared to a control.

54. The method of any of claims 50-53 wherein the subject has cancer.

55. The method of claim 54 wherein the cancer is selected from the group consisting of bladder, brain, breast, cervical, colo-rectal, esophageal, kidney, liver, lung, nasopharangeal, pancreatic, prostate, skin, stomach, uterine, ovarian, and testicular cancers.

56. A pharmaceutical composition comprising one or more agonists of a B7-H4 receptor comprising a neuropilin,
wherein the agonist induces or maintains signal transduction through the B7-H4 receptor.

57. The pharmaceutical composition of claim 56 wherein the agonist is a B7-H4 fusion protein.

58. The pharmaceutical composition of claim 57 wherein the B7-H4 fusion protein comprises a first domain comprising a soluble B7-H4 polypeptide and second domain.

59. The pharmaceutical composition of claim 58 wherein the second domain comprises an Ig Fc region.

60. The pharmaceutical composition of any of claims 58-59 wherein the soluble B7-H4 polypeptide comprises all or part of the extracellular domain of B7-H4.

61. The pharmaceutical composition of claim 60 wherein the soluble B7-H4 polypeptide consists of all or a part of the extracellular domain of B7-H4.

62. The pharmaceutical composition of claim 61 where the soluble B7-H4 polypeptide consists of the IgV domain of B7-H4.

63. The pharmaceutical composition of claim 57 wherein the fusion protein comprises the amino acid sequence of SEQ ID NO:22, 23, 24, or 25.

64. The pharmaceutical composition of claim 56 wherein the agonist is an anti-B7-H4 antibody or an antigen binding fragment thereof; an anti-B7-H4 receptor antibody or an antigen binding fragment thereof; a bi-specific antibody that targets B7-H4 and the B7-H4 receptor; or a combination thereof that induces, increases or enhances signal transduction through the B7-H4 receptor.

65. The pharmaceutical composition of claim 64 wherein the antibody or antigen binding fragment thereof binds to the extracellular domain of SEQ ID NO:1 or a functional fragment or variant thereof comprising at least 80% sequence identity to the extracellular domain of SEQ ID NO:1.

66. The pharmaceutical composition of claim 65 wherein the extracellular domain comprises amino acids 22-856 of SEQ ID NO:1, or a functional fragment thereof.

67. The pharmaceutical composition of claim 64 wherein the antibody or antigen binding fragment thereof binds to the extracellular domain of SEQ ID NO:2 or a functional fragment or variant thereof comprising at least 80% sequence identity to the extracellular domain of SEQ ID NO:2.

68. The pharmaceutical composition of claim 67 wherein the extracellular domain comprises amino acids 24-1894 of SEQ ID NO:2, or a functional fragment thereof.

69. The pharmaceutical composition of any of claims 56-68 further comprising a semaphorin protein, or a functional fragment thereof.

70. The pharmaceutical composition of claim 69 wherein the semaphorin is Sema3A, Sema6C, or a combination thereof.

71. The pharmaceutical composition of any of claims 56-70 wherein the agonist is present in an amount effective to inhibit or reduce an immune stimulatory response.

72. The pharmaceutical composition of any of claims 56-71 wherein the agonist is present in an amount effective to induce, increase, or enhance an immune inhibitory response.

73. The pharmaceutical composition of any of claims 56-72 wherein the composition comprises two more agonists.

74. The pharmaceutical composition of any of claims 50-73 wherein the agonist is in a dosage effective to decrease T cell response, proliferation of T cells, differentiation or effector function of T cells or survival of T cells.

75. The pharmaceutical composition of any of claims 56-74 wherein the agonist is in a dosage effective to promote or increase a regulatory T cell response, proliferation of regulatory T cells, differentiation or effector function of regulatory T cells or survival of regulatory T cells.

76. A method for enhancing Treg responses in a subject comprising administering to the subject the pharmaceutical composition of any of claims 56-75.

77. A method for treating or inhibiting one or more symptoms of an inflammatory response in a subject comprising administering to the subject the pharmaceutical composition of any of claims 56-75.

78. The method of claim 77, wherein the inflammatory response is associated with an autoimmune disease or disorder.

79. The method of claim 78, wherein the individual has an autoimmune disease selected from the group consisting of rheumatoid arthritis, systemic lupus erythematosus, alopecia areata, anklosing spondylitis, antiphospholipid syndrome, autoimmune addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

80. A method for reducing or inhibiting transplant rejection in an individual in need thereof comprising administering to the individual the pharmaceutical composition of any of claims 56-75.

81. A method for treating one or more symptoms of graft versus host disease (GVHD) in a subject comprising administering to a subject who has received or will receive a transplant the pharmaceutical composition of any of claims 56-75.

82. The composition of any of claim 12-39 or 56-75 or the method of any of claim 1-11, 40-55 or 76-81 wherein the antagonist or agonist is targeted to cells expressing the B7-H4 receptor.

83. A method of inducing or re-establishing immune tolerance comprising administering to a subject in need thereof an effective amount of a B7-H4-Ig fusion protein to modulate pro-inflammatory dendritic cell response or Th/Treg cell differentiation/balance in the subject in the subject.

84. The method of claim 83 wherein interaction between dendritic cells and Tregs is increased.

85. The method of any of claims 83-84 wherein the dendritic cells are mature dendritic cells.

86. The method of any of claims 83-85 wherein the interaction between the dendritic cells and the Th cells is dependent on a B7-H4 receptor comprising a neuropilin.

87. The method of any of claims 83-86 wherein Th activity is reduced.

88. The method of any of claims 83-87 wherein Treg activity is increased.

89. The method of any of claims 83-88 wherein the ratio of Treg activity to Th cell activity is increased.

90. The method of any of claims 83-89 wherein the subject has an inflammatory or autoimmune disease/disorder.

91. The method of claim 90 wherein one or more symptoms of the inflammatory or autoimmune disease/disorder are reduced.

92. The method of any of claims 83-91 wherein the Th cells in the subject overexpress a B7-H4 receptor comprising a neuropilin compared to Th cells in a control subject.

93. The method of any of claims 83-92 wherein the Th cells have greater cell surface expression of the B7-H4 receptor than the Treg cells.

94. The method of any of claims 83-93 wherein immune tolerance is induced or re-established in the subject.

95. The method of any of claims 83-94 wherein a B7-H4 fusion protein has lower affinity for the B7-H4 receptor thereof than anti-B7-H4 receptor antibody resulting in preferential binding to B7-H4 receptor high expressing Th cells, without compromising the ability of B7-H4 expressing dendritic cells to bind B7-H4 receptor low expressing Tregs.

96. The method of any of claims 83-95 wherein the B7-H4-Ig fusion protein comprises the amino acid sequence of SEQ ID NO:22, 23, 24, or 25.

97. A method for treating diseases associated with elevated levels of a B7-H4 receptor comprising a B7-H4 receptor protein, in a subject comprising administering to the subject the pharmaceutical composition of any of claims 50-69.

98. The method of claim 91, wherein the B7-H4 receptor protein comprises a neuropilin, a semaphorin, a plexin or a combination thereof.

99. A method for treating reduced expression of a B7-H4 receptor comprising a neuropilin, in a subject comprising administering to the subject the pharmaceutical composition of any of claims 56-75.


SEQUENCE LISTING (94 sequences)

SEQ ID NO: 1 - protein, 923 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFVPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPILITIIAMSALGVLLGAVCGVVLYCACWHNGMSERNLSALENYNFELVDGVKLKKDKLNTQSTYSEA

SEQ ID NO: 2 - protein, 1894 aa, Homo sapiens
MKAMPWNWTCLLSHLLMVGMGSSTLLTRQPAPLSQKQRSFVTFRGEPAEGFNHLVVDERTGHIYLGAVNRIYKLSSDLKVLVTHETGPDEDNPKCYPPRIVQTCNEPLTTTNNVNKMLLIDYKENRLIACGSLYQGICKLLRLEDLFKLGEPYHKKEHYLSGVNESGSVFGVIVSYSNLDDKLFIATAVDGKPEYFPTISSRKLTKNSEADGMFAYVFHDEFVASMIKIPSDTFTIIPDFDIYYVYGFSSGNFVYFLTLQPEMVSPPGSTTKEQVYTSKLVRLCKEDTAFNSYVEVPIGCERSGVEYRLLQAAYLSKAGAVLGRTLGVHPDDDLLFTVFSKGQKRKMKSLDESALCIFILKQINDRIKERLQSCYRGEGTLDLAWLKVKDIPCSSALLTIDDNFCGLDMNAPLGVSDMVRGIPVFTEDRDRMTSVIAYVYKNHSLAFVGTKSGKLKKIRVDGPRGNALQYETVQVVDPGPVLRDMAFSKDHEQLYIMSERQLTRVPVESCGQYQSCGECLGSGDPHCGWCVLHNTCTRKERCERSKEPRRFASEMKQCVRLTVHPNNISVSQYNVLLVLETYNVPELSAGVNCTFEDLSEMDGLVVGNQIQCYSPAAKEVPRIITENGDHHVVQLQLKSKETGMTFASTSFVFYNCSVHNSCLSCVESPYRCHWCKYRHVCTHDPKTCSFQEGRVKLPEDCPQLLRVDKILVPVEVIKPITLKAKNLPQPQSGQRGYECILNIQGSEQRVPALRFNSSSVQCQNTSYSYEGMEINNLPVELTVVWNGHFNIDNPAQNKVHLYKCGAMRESCGLCLKADPDFACGWCQGPGQCTLRQHCPAQESQWLELSGAKSKCTNPRITEIIPVTGPREGGTKVTIRGENLGLEFRDIASHVKVAGVECSPLVDGYIPAEQIVCEMGEAKPSQHAGFVEICVAVCRPEFMARSSQLYYFMTLTLSDLKPSRGPMSGGTQVTITGTNLNAGSNVVVMFGKQPCLFHRRSPSYIVCNTTSSDEVLEMKVSVQVDRAKIHQDLVFQYVEDPTIVRIEPEWSIVSGNTPIAVWGTHLDLIQNPQIRAKHGGKEHINICEVLNATEMTCQAPALALGPDHQSDLTERPEEFGFILDNVQSLLILNKTNFTYYPNPVFEAFGPSGILELKPGTPIILKGKNLIPPVAGGNVKLNYTVLVGEKPCTVTVSDVQLLCESPNLIGRHKVMARVGGMEYSPGMVYIAPDSPLSLPAIVSIAVAGGLLIIFIVAVLIAYKRKSRESDLTLKRLQMQMDNLESRVALECKEAFAELQTDIHELTSDLDGAGIPFLDYRTYTMRVLFPGIEDHPVLRDLEVPGYRQERVEKGLKLFAQLINNKVFLLSFIRTLESQRSFSMRDRGNVASLIMTVLQSKLEYATDVLKQLLADLIDKNLESKNHPKLLLRRTESVAEKMLTNWFTFLLYKFLKECAGEPLFSLFCAIKQQMEKGPIDAITGEARYSLSEDKLIRQQIDYKTLVLSCVSPDNANSPEVPVKILNCDTITQVKEKILDAIFKNVPCSHRPKAADMDLEWRQGSGARMILQDEDITTKIENDWKRLNTLAHYQVPDGSVVALVSKQVTAYNAVNNSTVSRTSASKYENMIRYTGSPDSLRSRTPMITPDLESGVKMWHLVKNHEHGDQKEGDRGSKMVSEIYLTRLLATKGTLQKFVDDLFETIFSTAHRGSALPLAIKYMFDFLDEQADKHGIHDPHVRHTWKSNCLPLRFWVNMIKNPQFVFDIHKNSITDACLSVVAQTFMDSCSTSEHRLGKDSPSNKLLYAKDIPSYKNWVERYYSDIGKMPAISDQDMNAYLAEQSRMHMNEFNTMSALSEIFSYVGKYSEEILGPLDHDDQCGKQKLAYKLEQVITLMSLDS

SEQ ID NO: 3 - DNA, 849 bp, Homo sapiens
atggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataa

SEQ ID NO: 4 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 5 - protein, 255 aa, Homo sapiens
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 6 - protein, 282 aa, Homo sapiens
MASLGQILFWSIISIIIILAGAIALIIGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 7 - protein, 255 aa, Homo sapiens
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 8 - protein, 129 aa, Artificial Sequence
  Synthetic polypeptide - IgV domain
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVN

SEQ ID NO: 9 - protein, 112 aa, Artificial Sequence
  Synthetic Polypeptide - IgV domain
HSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYK

SEQ ID NO: 10 - protein, 129 aa, Artificial Sequence
  Synthetic Polypeptide IgV domain
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVN

SEQ ID NO: 11 - protein, 112 aa, Artificial Sequence
  Synthetic Polypeptide - IgV domain
HSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYK

SEQ ID NO: 12 - protein, 85 aa, Artificial Sequence
  Synthetic Polypeptide - IgC domain
VDYNASSETLRCEAPRWFPQPTVVWASQVDQGANFSEVSNTSFELNSENVTMKVVSVLYNVTINNTYSCMIENDIAKATGDIKVT

SEQ ID NO: 13 - protein, 89 aa, Artificial Sequence
  Synthetic Polypeptide - IgC domain
PEVNVDYNASSETLRCEAPRWFPQPTVVWASQVDQGANFSEVSNTSFELNSENVTMKVVSVLYNVTINNTYSCMIENDIAKATGDIKVT

SEQ ID NO: 14 - protein, 100 aa, Artificial Sequence
  Synthetic Polypeptide - IgC domain
FSMPEVNVDYNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 15 - protein, 95 aa, Artificial Sequence
  Synthetic Polypeptide - IgC domain
VNVDYNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 16 - protein, 91 aa, Artificial Sequence
  Synthetic Polypeptide - IgC domain
YNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 17 - protein, 222 aa, Artificial Sequence
  Synthetic Polypeptide - extracellular domain
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 18 - protein, 222 aa, Artificial Sequence
  Synthetic Polypeptide - extracellular domain
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 19 - protein, 106 aa, Artificial Sequence
  Synthetic Polypeptide - IgC domain
EVNVDYNASSETLRCEAPRWFPQPTVVWASQVDQGANFSEVSNTSFELNSENVTMKVVSVLYNVTINNTYSCMIENDIAKATGDIKVTESEIKQQSHLQLLNSKAS

SEQ ID NO: 20 - protein, 356 aa, Artificial Sequence
  Synthetic Polypeptide - fusion protein
MEWSWVFLFFLSVTTGVHSEVNVDYNASSETLRCEAPRWFPQPTVVWASQVDQGANFSEVSNTSFELNSENVTMKVVSVLYNVTINNTYSCMIENDIAKATGDIKVTESEIKQQSHLQLLNSKASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 337 aa, Artificial Sequence
  Synthetic Polypeptide - fusion protein
EVNVDYNASSETLRCEAPRWFPQPTVVWASQVDQGANFSEVSNTSFELNSENVTMKVVSVLYNVTINNTYSCMIENDIAKATGDIKVTESEIKQQSHLQLLNSKASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 473 aa, Artificial Sequence
  Synthetic Polypeptide - fusion protein
MEWSWVFLFFLSVTTGVHSGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 454 aa, Artificial Sequence
  Synthetic Polypeptide - fusion protein
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 473 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 454 aa, Artificial Sequence
  Synthetic Polypeptide - fusion protein
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 371 aa, Artificial Sequence
  Synthetic Polypeptide - fusion protein
MEWSWVFLFFLSVTTGVHSFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 352 aa, Artificial Sequence
  Synthetic Polypeptide - fusion protein
FGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 376 aa, Artificial Sequence
  Synthetic Polypeptide - fusion protein
MEWSWVFLFFLSVTTGVHSFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 357 aa, Artificial Sequence
  Synthetic Polypeptide - fusion protein
FGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 380 aa, Artificial Sequence
  Synthetic Polypeptide - fusion protein
MEWSWVFLFFLSVTTGVHSFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 361 aa, Artificial Sequence
  Synthetic Polypeptide - fusion protein
FGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 478 aa, Artificial Sequence
  Synthetic Polypeptide - fusion protein
MEWSWVFLFFLSVTTGVHSFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 33 - protein, 459 aa, Artificial Sequence
  Synthetic Polypeptide - fusion protein
FGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 34 - protein, 482 aa, Artificial Sequence
  Synthetic Polpypeptide - fusion protein
MEWSWVFLFFLSVTTGVHSGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 35 - protein, 463 aa, Artificial Sequence
  Synthetic Polypeptide - fusion protein
GFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 36 - DNA, 1449 bp, Artificial Sequence
  Synthetic Polynucleotide - encodes a fusion protein
atggagtggtcatgggtttttctgttctttcttagcgtgactacaggcgtccattcaggattcggcataagcggcaagcacttcatcacagttacaacgtttacaagtgcggggaacattggggaagatggaacattgtcatgtacatttgagccagatatcaaactcaatggaatagtaattcagtggcttaaggagggcatcaagggcctggtccacgaatttaaggaggggaaagacgatctgtctcagcagcacgagatgttcaggggcagaaccgccgtcttcgcagaccaggttgtggtaggcaacgccagtttgcggctgaaaaacgtgcagctgactgacgccggcacctacacatgctatatccggtcctctaagggcaaggggaacgctaatctcgagtacaaaacaggcgccttttctatgccagagatcaacgtggactataacgcaagctctgaaagtctgagatgcgaggcgccaaggtggttccctcagcccaccgtcgcgtgggcttcccaggtggatcaaggcgccaacttttctgaggtttctaacaccagcttcgaactgaacagcgaaaatgtgacaatgaaggtagtcagcgttctgtataacgtgaccatcaacaatacttactcctgtatgatagaaaatgatatagccaaggctacaggagatattaaagtgacggattcagaagtgaaaaggaggagtcaactgcaactcttgaatagcggcgagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaatga

SEQ ID NO: 37 - protein, 906 aa, Homo sapiens [sequence omitted]

SEQ ID NO: 38 - protein, 931 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIRFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 39 - protein, 901 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 40 - protein, 930 aa, Homo sapiens
MPRAPHFMPLLLLLLLLSLPHTQAAFPQDPLPLLISDLQGTSPLSWFRGLEDDAVAAELGLDFQRFLTLNRTLLVAARDHVFSFDLQAEEEGEGLVPNKYLTWRSQDVENCAVRGKLTDECYNYIRVLVPWDSQTLLACGTNSFSPVCRSYGITSLQQEGEELSGQARCPFDATQSNVAIFAEGSLYSATAADFQASDAVVYRSLGPQPPLRSAKYDSKWLREPHFVQALEHGDHVYFFFREVSVEDARLGRVQFSRVARVCKRDMGGSPRALDRHWTSFLKLRLNCSVPGDSTFYFDVLQALTGPVNLHGRSALFGVFTTQTNSIPGSAVCAFYLDEIERGFEGKFKEQRSLDGAWTPVSEDRVPSPRPGSCAGVGGAALFSSSRDLPDDVLTFIKAHPLLDPAVPPVTHQPLLTLTSRALLTQVAVDGMAGPHSNITVMFLGSNDGTVLKVLTPGGRSGGPEPILLEEIDAYSPARCSGKRTAQTARRIIGLELDTEGHRLFVAFSGCIVYLPLSRCARHGACQRSCLASQDPYCGWHSSRGCVDIRGSGGTDVDQAGNQESMEHGDCQDGATGSQSGPGDSAYGVRRDLPPASASRSVPIPLLLASVAAAFALGASVSGLLVSCACRRAHRRRGKDIETPGLPRPLSLRSLARLHGGGPEPPPPSKDGDAVQTPQLYTTFLPPPEGVPPPELACLPTPESTPELPVKHLRAAGDPWEWNQNRNNAKEGPGRSRGGHAAGGPAPRVLVRPPPPGCPGQAVEVTTLEELLRYLHGPQPPRKGAEPPAPLTSRALPPEPAPALLGGPSPRPHECASPLRLDVPPEGRCASAPARPALSAPAPRLGVGGGRRLPFSGHRAPPALLTRVPSGGPSRYSGGPGKHLLYLGRPEGYRGRALKRVDVEKPQLSLKPPLVGPSSRQAVPNGGRFNF

SEQ ID NO: 41 - protein, 537 aa, Homo sapiens
MPRAPHFMPLLLLLLLLSLPHTQAAFPQDPLPLLISDLQGTSPLSWFRGLEDDAVAAELGLDFQRFLTLNRTLLVAARDHVFSFDLQAEEEGEGLVPNKYLTWRSQDVENCAVRGKLTDECYNYIRVLVPWDSQTLLACGTNSFSPVCRSYGITSLQQEGEELSGQARCPFDATQSNVAIFAEGSLYSATAADFQASDAVVYRSLGPQPPLRSAKYDSKWLREPHFVQALEHGDHVYFFFREVSVEDARLGRVQFSRVARVCKRDMGGSPRALDRHWTSFLKLRLNCSVPGDSTFYFDVLQALTGPVNLHGRSALFGVFTTQTNSIPGSAVCAFYLDEIERGFEGKFKEQRSLDGAWTPVSEDRVPSPRPGSCPAHSSPLGRSLQVLRGSREAPPVPGPARGLPGPRPEKGGRREAPVVPEASPRRALLPPGRPERRPFQLLKGAVHGLQRGERPSPLGHELDALQDVSPPPRPAPPAFPTRRVSRGPFSPRVYLLTVFPPVLDRRVGGEKPVSSVSQHFRGSWRTPTPRSLPAKLP

SEQ ID NO: 42 - protein, 474 aa, Homo sapiens [sequence omitted]

SEQ ID NO: 43 - protein, 771 aa, Homo sapiens [sequence omitted]

SEQ ID NO: 44 - protein, 468 aa, Artificial Sequence
  Synthetic Polypeptide - fusion protein
MEWSWVFLFFLSVTTGVHSGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 449 aa, Artificial Sequence
  Synthetic Polypeptide - fusion protein
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 464 aa, Artificial Sequence
  Synthetic Polypeptide - fusion protein
MEWSWVFLFFLSVTTGVHSGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 445 aa, Artificial Sequence
  Synthetic Polypeptide - fusion protein
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 476 aa, Artificial Sequence
  Synthetic Polypeptide - fusion protein
MEWSWVFLFFLSVTTGVHSGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 457 aa, Artificial Sequence
  Synthetic Polypeptide - fusion protein
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 468 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 449 aa, Artificial Sequence
  Synthetic Polypeptide - fusion protein
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 464 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 445 aa, Artificial Sequence
  Synthetic Polypeptide - fusion protein
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 476 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 457 aa, Artificial Sequence
  Synthetic Polypeptide - fusion protein
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 473 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 454 aa, Artificial Sequence
  Synthetic Polypeptide - fusion protein
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 481 aa, Artificial Sequence
  Synthetic Polypeptide - fusion protein
MEWSWVFLFFLSVTTGVHSFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKQQSHLQLLNSKASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 462 aa, Artificial Sequence
  Synthetic Polypeptide - fusion protein
FGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKQQSHLQLLNSKASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 343 aa, Artificial Sequence
  Synthetic Polypeptide - fusion protein
MEWSWVFLFFLSVTTGVHSEVNVDYNASSETLRCEAPRWFPQPTVVWASQVDQGANFSEVSNTSFELNSENVTMKVVSVLYNVTINNTYSCMIENDIAKATGDIKVTESEIKSEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 324 aa, Artificial Sequence
  Synthetic Polypeptide - fusion protein
EVNVDYNASSETLRCEAPRWFPQPTVVWASQVDQGANFSEVSNTSFELNSENVTMKVVSVLYNVTINNTYSCMIENDIAKATGDIKVTESEIKSEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 751 aa, Homo sapiens
NYQNGKNNVPRLKLSYKEMLESNNVITFNGLANSSSYHTFLLDEERSRLYVGAKDHIFSFDLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPKFISAHLISESDNPEDDKVYFFFRENAIDGEHSGKATHARIGQICKNDFGGHRSLVNKWTTFLKARLICSVPGPNGIDTHFDELQDVFLMNFKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPMNNRPIVIKTDVNYQFTQIVVDRVDAEDGQYDVMFIGTDVGTVLKVVSIPKETWYDLEEVLLEEMTVFREPTAISAMELSTKQQQLYIGSTAGVAQLPLHRCDIYGKACAECCLARDPYCAWDGSACSRYFPTAKRRTRRQDIRNGDPLTHCSDLHHDNHHGHSPEERIIYGVENSSTFLECSPKSQRALVYWQFQRRNEERKEEIRVDDHIIRTDQGLLLRSLQQKDSGNYLCHAVEHGFIQTLLKVTLEVIDTEHLEELLHKDDDGDGSKTKEMSNSMTPSQKVWYRDFMQLINHPNLNTMDEFCEQVWKRDRKQRRQRPGHTPGNSNKWKHLQENKKGRNRRTHEFERAPRSV

SEQ ID NO: 63 - protein, 644 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSGIK

SEQ ID NO: 64 - protein, 473 aa, Artificial Sequence; identical to SEQ ID NO: 24
  Synthetic Polypeptide - fusion protein

SEQ ID NO: 65 - protein, 473 aa, Artificial Sequence; identical to SEQ ID NO: 56
  Synthetic Polypeptide - fusion protein

SEQ ID NO: 66 - protein, 473 aa, Artificial Sequence; identical to SEQ ID NO: 24
  Synthetic Polypeptide - fusion protein

SEQ ID NO: 67 - protein, 473 aa, Artificial Sequence; identical to SEQ ID NO: 24
  Synthetic Polypeptide - fusion protein

SEQ ID NO: 68 - protein, 481 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 473 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 473 aa, Artificial Sequence; identical to SEQ ID NO: 69
  Synthetic Polypeptide - fusion protein

SEQ ID NO: 71 - protein, 473 aa, Artificial Sequence; identical to SEQ ID NO: 69
  Synthetic Polypeptide - fusion protein

SEQ ID NO: 72 - protein, 483 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKQQSHLQLLNSKASEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 483 aa, Artificial Sequence
  Synthetic Polypeptide - fusion protein
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASESKYGPKTHPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 74 - protein, 483 aa, Artificial Sequence; identical to SEQ ID NO: 72
  Synthetic Polypeptide - fusion protein

SEQ ID NO: 75 - protein, 118 aa, Artificial Sequence
  Synthetic Polypeptide - fragment of Nrp2
VDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 76 - protein, 93 aa, Artificial Sequence
  Synthetic Polypeptide - alternative sequence fragment of Nrp2
GGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 77 - protein, 118 aa, Artificial Sequence; identical to SEQ ID NO: 75
  Synthetic Polypeptide - fragment of Nrp2

SEQ ID NO: 78 - protein, 93 aa, Artificial Sequence; identical to SEQ ID NO: 76
  Synthetic Polypeptide - alternative sequence fragment of Nrp2

SEQ ID NO: 79 - protein, 118 aa, Artificial Sequence; identical to SEQ ID NO: 75
  Synthetic Polypeptide - fragment of Nrp2

SEQ ID NO: 80 - protein, 93 aa, Artificial Sequence; identical to SEQ ID NO: 76
  Synthetic Polypeptide - alternative sequence fragment of Nrp2

SEQ ID NO: 81 - protein, 8 aa, Artificial Sequence
  Synthetic Polypeptide - fragment of Nrp2
LFEGNMHY

SEQ ID NO: 82 - protein, 8 aa, Artificial Sequence
  Synthetic Polypeptide - alternative sequence fragment of Nrp2
VGCSWRPL

SEQ ID NO: 83 - protein, 65 aa, Artificial Sequence
  Synthetic Polypeptide - fragment of plexin4A
IRVDGPRGNALQYETVQVVDPGPVLRDMAFSKDHEQLYIMSERQLTRVPVESCGQYQSCGECLGS

SEQ ID NO: 84 - protein, 65 aa, Artificial Sequence
  Synthetic Polypeptide - alternative sequence for fragment of plexin4A
SFGTGPQGGITQEWIGVEGDPPGANIASQEQMLCVYLQCSSHKAISDQRVQPLLCCFLNVPGNSS

SEQ ID NO: 85 - protein, 35 aa, Artificial Sequence
  Synthetic Polypeptide - fragment of plexin4A
IRVDGPRGNALQYETVQVVDPGPVLRDMAFSKDHE

SEQ ID NO: 86 - protein, 35 aa, Artificial Sequence
  Synthetic Polypeptide - alternative sequence fragment of plexin4A
MPGTSLCPTLELQTGPRSHRATVTLELLFSSCSSN

SEQ ID NO: 87 - protein, 35 aa, Artificial Sequence; identical to SEQ ID NO: 85
  Synthetic Polypeptide - fragment of plexin4A

SEQ ID NO: 88 - protein, 35 aa, Artificial Sequence; identical to SEQ ID NO: 86
  Synthetic Polypeptide - alternative sequence of fragment of plexin4A

SEQ ID NO: 89 - protein, 65 aa, Artificial Sequence; identical to SEQ ID NO: 83
  Synthetic Polypeptide - fragment of plexin4A

SEQ ID NO: 90 - protein, 65 aa, Artificial Sequence; identical to SEQ ID NO: 84
  Synthetic Polypeptide - alternative sequence fragment of plexin4A

SEQ ID NO: 91 - protein, 33 aa, Artificial Sequence
  Synthetic Polypeptide - alternative sequence fragment of sema6C
YVLPGPGPSPGTPSPPSDAHPRPQSSTLGVHTR

SEQ ID NO: 92 - protein, 33 aa, Artificial Sequence; identical to SEQ ID NO: 91
  Synthetic Polypeptide - alternative sequence fragment of sema6C

SEQ ID NO: 93 - protein, 33 aa, Artificial Sequence; identical to SEQ ID NO: 91
  Synthetic Polypeptide - alternative sequence fragment of sema6C

SEQ ID NO: 94 - protein, 6 aa, Artificial Sequence
  Synthetic Polypeptide - linker
IEGRMD